(19) United States
(12) Patent Application Publication
(10) Pub. No.: US 2024/0360473 A1
(43) Pub. Date: Oct. 31, 2024

(54) Compositions and Methods for AAV Production

(71) Applicant: Shape Therapeutics Inc., Seatle, WA (US)

(72) Inventors: Kenneth Prentice, Seattle, WA (US); Sandhya Pande, Kent, WA (US)

(21) Appl. No.: 18/600,530

(22) Filed: Mar. 8, 2024

Related U.S. Application Data
(60) Provisional application No. 63/450,898, filed on Mar. 8, 2023.

(51) Int. Cl. C12N 15/86

(52) U.S. Cl. CPC C12N 15/86; C12N 2750/14143; C12N 2800/60

(57) ABSTRACT

Described herein are compositions and methods of using stable cells lines in combination with transient transfection to produce recombinant AAV (rAAV).

### CROSS-REFERENCE TO RELATED APPLICATIONS

This application claims priority benefit of U.S. Provisional Application No. 63/450,898, filed Mar. 8, 2023, the disclosure of which is incorporated herein by reference in its entirety.

### INCORPORATION BY REFERENCE OF SEQUENCE LISTING PROVIDED AS A TEXT FILE

A Sequence Listing is provided herewith as a text file, “SHPE-013_Seq List.xml,” created on Mar. 6, 2024 and having a size of 537,297 bytes. The contents of the text file are incorporated by reference herein in their entirety.

### BACKGROUND

Recombinant adeno-associated virus (rAAV) is the preferred vehicle for in vivo gene delivery. AAV has no known disease associations, infects dividing and non-dividing cells, rarely if ever integrates into the mammalian cell genome, and can persist essentially for the lifetime of infected cells as a transcriptionally active nuclear episome. The FDA has recently approved several rAAV gene therapy products and many other rAAV-based gene therapy and gene editing products are in development.

The most widely used method for producing rAAV virions is based on the helper-virus-free transient transfection of multiple plasmids, typically a triple transfection, into adherent cell lines. Although there is ongoing investment to increase production capacity, current AAV manufacturing processes are inefficient and expensive. In addition, they result in variable product quality, with low levels of encapsidation of a payload, such as a therapeutic payload.

There is, therefore, a need for improved methods for producing rAAV products. Any such solution must address the toxicity to the host production cell due to constitutive expression of AAV Rep protein and the toxicity to the host production cell due to constitutive expression of adenoviral helper protein.

### SUMMARY

Disclosed herein are cells are capable of conditionally producing recombinant AAV (rAAV) virions within which are packaged an expressible payload. Disclosed herein are polynucleotide constructs that are capable of conditionally producing recombinant AAV (rAAV) virions within which are packaged an expressible payload, when introduced into a cell. The polynucleotide constructs may or may not be integrated into the genome of the cell. One or more polynucleotide constructs may be integrated into the genome of the cell and/or one or more polynucleotide constructs may be present in the cell as plasmids.

The cells capable of conditionally producing recombinant AAV (rAAV) virions within which are packaged an expressible payload may be a stable cell line. The stable cell line may be a stable AAV helper cell line, wherein an AAV helper construct is integrated into the genome of the cell. The stable cell line may be a stable Rep/Cap cell line, wherein a Rep/Cap construct is integrated into the genome of the cell. The stable cell line may be a payload stable cell line, wherein a payload construct is integrated into the genome of the cell. The stable cell line may further comprise one or more polynucleotide constructs, wherein the one or more polynucleotide constructs may not be integrated into the genome, e.g., may be present in plasmids.

Provided herein is a stable cell line that may be transiently transfected with one or more plasmids. Further provided herein is a stable cell line that may be transiently transfected with one or more plasmids, wherein the cells are capable of conditionally producing recombinant AAV (rAAV) virions within which are packaged an expressible payload; and wherein a population of virions produced by the stable cell are more homogenous than a population of virions produced by an otherwise comparable cell producing rAAV virions upon transient transfection. The stable cell line may be a stable AAV helper cell line that is transiently transfected with a Rep/Cap plasmid and a payload plasmid. The stable cell line may be a stable Rep/Cap cell line that is transiently transfected with a AAV helper plasmid and a payload plasmid. The stable cell line may be a payload stable cell line that is transiently transfected with a Rep/Cap plasmid and an AAV helper plasmid.

Further provided herein is a stable cell line that may be transiently transfected with one or more plasmids, wherein the cells are capable of conditionally producing recombinant AAV (rAAV) virions within which are packaged an expressible payload; and production of virions is inducible upon addition of an activator and at least one triggering agent. The stable cell line may be a stable AAV helper cell line that is transiently transfected with a Rep/Cap plasmid and a payload plasmid. The stable cell line may be a stable Rep/Cap cell line that is transiently transfected with a AAV helper plasmid and a payload plasmid. The stable cell line may be a payload stable cell line that is transiently transfected with a Rep/Cap plasmid and an AAV helper plasmid.

In some aspects, a cell for inducibly producing recombinant adenovirus associated virus (rAAV) virions comprises: (a) a first plasmid comprising a first polynucleotide construct comprising an AAV Rep coding sequence, a sequence encoding one or more AAV capsid proteins, and a first constitutive promoter operably linked to a sequence encoding a first selectable marker, optionally, wherein the first polynucleotide construct comprises from 5′ to 3′: one or more promoters operably linked to a first sequence comprising a first part of the AAV Rep coding sequence; a second sequence comprising a second part of the AAV Rep coding sequence, wherein the one or more promoters are not operably linked to the second sequence comprising the second part of the AAV Rep coding sequence, wherein the first sequence and the second sequence are separated by (i) an excisable element that comprises a first recombination site and a second recombination site flanking a coding sequence encoding a stop signaling sequence, wherein the first recombination site and the second recombination site are oriented in the same direction or (ii) an inversible element that comprises a first recombination site and a second recombination site flanking a coding sequence encoding a stop signaling sequence, wherein the first recombination site and the second recombination site are oriented in opposite directions; and a third sequence comprising the sequence encoding one or more AAV capsid proteins, wherein the second sequence comprises a promoter that is operably linked to the third sequence; and the first constitutive promoter operably linked to a sequence encoding the first selectable marker; (b) a second polynucleotide construct integrated into the nuclear genome of the cell, comprising from 5′ to 3′: a second constitutive promoter operably linked to a sequence encoding an activator, wherein the cell constitutively expresses the activator and the activator is unable to activate an inducible promoter in absence of a first triggering agent; the inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; a sequence encoding one or more AAV helper proteins, wherein the inducible promoter is not operably linked to the sequence encoding the one or more AAV helper proteins; and a third constitutive promoter operably linked to a sequence encoding a second selectable marker, wherein the cell constitutively expresses the second selectable marker; and (c) a second plasmid comprising a third polynucleotide construct comprising a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR); wherein in absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence. In some embodiments, the payload is progranulin or dystrophin; optionally, wherein the dystrophin is a short, functional dystrophin. In some embodiments, the coding sequence encoding the stop signaling sequence further encodes a protein marker that comprises the stop signaling sequence. In some embodiments, the cell further comprises: an adenovirus ElA protein and ElB protein, and the one or more AAV helper proteins expressed by the second polynucleotide construct are an adenovirus E2A protein and E4 protein, or an adenovirus E2A protein and E4 protein, and the one or more AAV helper proteins expressed by the second polynucleotide construct are an adenovirus ElA protein and ElB protein. In some embodiments, the cell further comprising a fourth polynucleotide construct comprising an inducible or constitutive promoter operably linked to a sequence encoding one or more helper proteins. In some embodiments, the sequence coding for one or more AAV helper proteins comprises a bicistronic open reading frame encoding two AAV helper proteins. In some embodiments, the two AAV helper proteins are E2A and E4 or ElA and ElB. In some embodiments, wherein the bicistronic open reading frame comprises an internal ribosome entry site (IRES) or a peptide 2A (P2A) sequence. In some embodiments, transcription of the AAV Rep coding sequences is driven by the P5 and P19 native AAV promoters, and transcription of the sequence encoding the one or more AAV capsid proteins is driven by the P40 native AAV promoter. In some embodiments, transcription of the AAV Rep coding sequences is driven by an inducible promoter and transcription of the sequence encoding the one or more AAV capsid proteins is driven by an inducible promoter. In some embodiments, the AAV capsid proteins comprise VP1, VP2, and VP3. In some embodiments, the cell is a mammalian cell; optionally, wherein the mammalian cell is a HEK293 cell. In some embodiments, the first polynucleotide construct and the third polynucleotide construct are not integrated into the nuclear genome of the cell. In some embodiments, the inducible promoter in the second polynucleotide construct is selected from the group consisting of a tetracycline-inducible promoter, an ecdysone-inducible promoter, and a cumate-inducible promoter. In some embodiments, the first triggering agent for inducing the tetracycline-inducible promoter is tetracycline or doxycycline. In some embodiments, the inducible recombinase is fused to an estrogen response element (ER) and translocates to the nucleus in the presence of tamoxifen. In some embodiments, the second triggering agent for translocating the inducible recombinase is a hormone, optionally, wherein the second triggering agent is tamoxifen. In some embodiments, the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase. In some embodiments, upon expression of the inducible recombinase in the presence of the second triggering agent, the inducible recombinase translocates to the nucleus, recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein, wherein the one or more promoters are operably linked to the complete AAV Rep coding sequence to allow expression of an AAV Rep protein and an AAV Cap protein. In some embodiments, the second polynucleotide construct further comprises an insert comprising a sequence encoding VA-RNA; or optionally, wherein a fifth construct comprises an insert comprising a sequence encoding VA-RNA. In some embodiments, the VA-RNA is wild-type VA-RNA or VA-RNA comprising one or more mutations in the VA-RNA internal promoter. In some embodiments, the insert comprises: a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and a VA-RNA coding sequence, wherein excision of the second excisable element by the inducible recombinase generates a functional complete second constitutive promoter operably linked to the VA-RNA coding sequence to allow expression of the VA-RNA. In some embodiments, the first part of the second constitutive promoter comprises a distal sequence element (DSE) of an RNA polymerase III promoter, and the second part of the second constitutive promoter comprises a proximal sequence element (PSE) of an RNA polymerase III promoter; the first part of the second constitutive promoter comprises a distal sequence element (DSE) of a U6 promoter, and the second part of the second constitutive promoter comprises a proximal sequence element (PSE) of a U6 promoter; or the first part of the second constitutive promoter comprises a distal sequence element (DSE) of a U7 promoter, and the second part of the second constitutive promoter comprises a proximal sequence element (PSE) of a U7 promoter. In some embodiments, the first polynucleotide construct further comprises: (i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or (ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site. In some embodiments, first part of the AAV Rep coding sequence comprises a p5 internal promoter and a p19 internal promoter, and the second part of the AAV Rep coding sequence comprises a p40 internal promoter. In some embodiments, the excisable spacer is inserted at an insertion site between the p19 internal promoter and the p40 internal promoter of the AAV Rep coding sequence. In some embodiments, the insertion site is between a CAG and a G, a CAG and an A, an AAG and a G, and an AAG and an A. In some embodiments, the first plasmid comprises at least 5%, 10%, 20%, 30%, 40%, 50%, 60%, 70%, 75%, 80%, 85%, 90%, 95%, or 100% sequence identity to SEQ ID NO: 1; SEQ ID NO: 2; SEQ ID NO: 3; SEQ ID NO: 4. In some embodiments, the first polynucleotide construct comprises at least 5%, 10%, 20%, 30%, 40%, 50%, 60%, 70%, 75%, 80%, 85%, 90%, 95%, or 100% sequence identity to SEQ ID NO: 1; SEQ ID NO: 2; SEQ ID NO: 3; SEQ ID NO: 4. In some embodiments, the second polynucleotide construct comprises at least 5%, 10%, 20%, 30%, 40%, 50%, 60%, 70%, 75%, 80%, 85%, 90%, 95%, or 100% sequence identity to SEQ ID NO: 5; SEQ ID NO: 6; SEQ ID NO: 7; SEQ ID NO: 8; or SEQ ID NO: 9-SEQ ID NO: 13. In some embodiments, the third polynucleotide construct comprises at least 5%, 10%, 20%, 30%, 40%, 50%, 60%, 70%, 75%, 80%, 85%, 90%, 95%, or 100% sequence identity to SEQ ID NO: 14; SEQ ID NO: 16; SEQ ID NO: 18; or SEQ ID NO: 20; optionally, wherein the sequence encoding progranulin is replace with a sequence encoding dystrophin; further optionally, wherein the sequence encoding dystrophin encodes for a short, functional dystrophin. In some embodiments, the second plasmid comprises at least 5%, 10%, 20%, 30%, 40%, 50%, 60%, 70%, 75%, 80%, 85%, 90%, 95%, or 100% sequence identity to SEQ ID NO: 15; SEQ ID NO: 17; SEQ ID NO: 19; or SEQ ID NO: 21; optionally, wherein the sequence encoding progranulin is replace with a sequence encoding dystrophin; further optionally, wherein the sequence encoding dystrophin encodes for a short, functional dystrophin. In some embodiments, the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 14; SEQ ID NO: 16; SEQ ID NO: 18; or SEQ ID NO: 20; optionally, wherein the sequence encoding progranulin is replace with a sequence encoding dystrophin; further optionally, wherein the sequence encoding dystrophin encodes for a short, functional dystrophin. In some embodiments, the third polynucleotide construct further comprises a spacer between the 5′ ITR and the sequence encoding the third selectable marker or a spacer between the sequence encoding the third selectable marker and the 3′ ITR, or a combination thereof. In some embodiments, the spacer ranges in length from 500 base pairs to 5000 base pairs.

In some aspects, a system for inducibly producing recombinant adenovirus associated virus (rAAV) virions comprises: (a) a first plasmid according to any one of the embodiments disclosed herein; (b) a cell comprising the second polynucleotide construct integrated into the nuclear genome of the cell according to any one of the embodiments disclosed herein; and (c) a second plasmid according to any one of the embodiments disclosed herein; optionally further comprising (d) a fourth polynucleotide construct according to any one of the embodiments disclosed herein; and further optionally comprising a fifth polynucleotide construct according to any one of the embodiments disclosed herein.

In some aspects, a system for inducibly producing recombinant adenovirus associated virus (rAAV) virions comprises: (a) the first polynucleotide construct in the first plasmid according to any one of the embodiments disclosed herein; (b) a cell comprising the second polynucleotide construct integrated into the nuclear genome of the cell according to any one of the embodiments disclosed herein; and (c) the third polynucleotide construct in the second plasmid according to any one of the embodiments disclosed herein; optionally further comprising (d) a fourth polynucleotide construct according to any one of the embodiments disclosed herein; and further optionally comprising a fifth polynucleotide construct according to any one of the embodiments disclosed herein. In some embodiments, the system further comprises the first triggering agent; optionally, wherein the first triggering agent is doxycycline. In some embodiments, the system further comprises the second triggering agent; optionally, wherein the second triggering agent is tamoxifen.

In some aspects, a method of generating a cell for inducibly producing recombinant AAV (rAAV) virions comprising a payload comprises: introducing into a cell a second polynucleotide construct according to any one of the embodiments disclosed herein; selecting for cells expressing the second selectable marker; introducing into a cell of the cells expressing the second selectable marker the first polynucleotide construct according to any one of the embodiments disclosed herein and the third polynucleotide construct according to any one of the embodiments disclosed herein, optionally, wherein the introducing is via transient transfection; thereby generating the cell inducibly producing recombinant AAV (rAAV) virions comprising a payload wherein the first polynucleotide construct is integrated into the nuclear genome of the cell and the first polynucleotide construct and the third polynucleotide construct are not integrated into the genome of the cell.

In some aspects, a method of generating a cell for inducibly producing recombinant AAV (rAAV) virions comprising a payload comprises: introducing into a cell a second polynucleotide construct according to any one of the embodiments disclosed herein; selecting for cells expressing the second selectable marker; introducing into a cell of the cells expressing the second selectable marker the first plasmid according to any one of the embodiments disclosed herein and the second plasmid according to any one of claims the embodiments disclosed herein, optionally, wherein the introducing is via transient transfection; thereby generating the cell inducibly producing recombinant AAV (rAAV) virions comprising a payload wherein the first polynucleotide construct is integrated into the nuclear genome of the cell and the first plasmid and the plasmid are not integrated into the genome of the cell. In some embodiments, the method further comprising contacting the cell for inducibly producing recombinant AAV (rAAV) virions comprising a payload to the first triggering agent and the second triggering agent, wherein in the presence of the first triggering agent, the activator activates the inducible promoter resulting in expression of the inducible recombinase, wherein in the presence of the second triggering agent, the inducible recombinase translocates to the nucleus, wherein recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; thereby inducibly producing recombinant AAV (rAAV) virions comprising a payload; optionally, further comprising (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein, wherein the one or more promoters are operably linked to the complete AAV Rep coding sequence to allow expression of an AAV Rep protein and an AAV Cap protein. In some embodiments, the cell is a mammalian cell. In some embodiments, the mammalian cell is a HEK293 cell. In some embodiments, the first polynucleotide construct is in a plasmid comprising at least 70%, 75%, 80%, 85%, 90%, 95%, or 100% sequence identity to SEQ ID NO: 3. In some embodiments, the second polynucleotide construct is in a plasmid comprising at least 70%, 75%, 80%, 85%, 90%, 95%, or 100% sequence identity to SEQ ID NO: 12. In some embodiments, the third polynucleotide construct is in a plasmid comprising at least 70%, 75%, 80%, 85%, 90%, 95%, or 100% sequence identity to SEQ ID NO: 15; optionally, wherein the sequence encoding progranulin is replaced with a sequence encoding dystrophin; further optionally, wherein the sequence encoding dystrophin encodes for a short, functional dystrophin. In some embodiments, the third polynucleotide construct is in a plasmid comprising at least 70%, 75%, 80%, 85%, 90%, 95%, or 100% sequence identity to SEQ ID NO: 17; optionally, wherein the sequence encoding progranulin is replaced with a sequence encoding dystrophin; further optionally, wherein the sequence encoding dystrophin encodes for a short, functional dystrophin. In some embodiments, the third polynucleotide construct is in a plasmid comprising at least 70%, 75%, 80%, 85%, 90%, 95%, or 100% sequence identity to SEQ ID NO: 19; optionally, wherein the sequence encoding progranulin is replaced with a sequence encoding dystrophin; further optionally, wherein the sequence encoding dystrophin encodes for a short, functional dystrophin. In some embodiments, the third polynucleotide construct is in a plasmid comprising at least 70%, 75%, 80%, 85%, 90%, 95%, or 100% sequence identity to SEQ ID NO: 21.

In some aspects, a method for generating a recombinant adenovirus associated virus (rAAV) virion comprising a sequence encoding a payload comprises contacting the cell according to any one of the embodiments disclosed herein with the first triggering agent and the second triggering agent, wherein in the presence of the first triggering agent, the activator activates the inducible promoter of the second polynucleotide construct resulting in expression of the inducible recombinase, wherein in the presence of the second triggering agent, the inducible recombinase translocates to the nucleus, wherein recombination between the first recombination site and the second recombination site in the first polynucleotide construct by the inducible recombinase results in excision of the excisable element or inversion of the inversible element, wherein the inducible promoter becomes operably linked to the sequence encoding the one or more AAV helper proteins to allow expression of the one or more AAV helper proteins; and optionally, the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein, and recombination between the third recombination site and the fourth recombination site in the second polynucleotide construct by the inducible recombinase results in excision of the self-excising element comprising the sequence encoding the inducible recombinase, wherein the expression of the one or more AAV helper proteins results in expression of the one or more Rep proteins and the one or more capsid proteins, thereby generating an rAAV virion comprising the sequence encoding the payload of interest. In some embodiments, the payload is progranulin or dystrophin; optionally, wherein the dystrophin is a short, functional dystrophin. In some embodiments, the first triggering agent is doxycycline. In some embodiments, the second triggering agent is tamoxifen.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A depicts an exemplary system of polynucleotides for inducibly producing rAAV. This depiction is referred to as a v1.0 system. In absence of first and second triggering agents, the system is in an off state. This system includes Construct 1, Construct 2, and Construct 3. Construct 1 inducibly expresses AAV Cap proteins and inducibly expresses full-length AAV Rep proteins, and both AAV Cap proteins and AAV Rep proteins are driven by their native promoters (p40, and p5 and p19, respectively).

FIG. 1B depicts the post-triggered state (also referred to as the on state) of Constructs 1-3 shown in FIG. 1A following the addition of the first triggering agent, doxycycline, and the second triggering agent, tamoxifen. The Cre coding element is positioned between LoxP sites and is additionally fused to estrogen response elements (“ER2”), which allows for control over the localization of Cre in response to estrogen agonists, such as tamoxifen. Upon addition of a first triggering agent, e.g., doxycycline, Cre is expressed, and upon addition of the second triggering agent, e.g., tamoxifen, Cre translocates to the nucleus. Following translation and translocation of the Cre protein into the cell nucleus, the Cre protein effects excision of its own coding sequence, leaving the integrated construct 2 as shown in FIG. 1B. Therefore, adenoviral E2A and E4 helper proteins are expressed. Cre also excises out the excisable element flanked by LoxP sites in Construct 1, allowing expression of AAV Rep and Cap proteins under control of native promoters. rAAV virions encapsidating the payload, such as a GOI, are therefore produced.

FIG. 2A depicts an exemplary system of polynucleotides for inducibly producing rAAV, which is also an exemplary v1.2 system. In absence of a first triggering agent, e.g., doxycycline, and a second triggering agent, e.g., tamoxifen, the system is in an off state. In Construct 1 of this schematic, the sequence encoding the AAV Cap proteins is operably linked to an inducible promoter (for example, Tet-On promoter). This is different from the v1.0 system in which the AAV Cap proteins are expressed under the control of a native AAV promoter. In this v1.2 system, the coding sequences for the Rep and Cap proteins are oriented in opposite directions such that the internal p40 promoter in Rep coding sequence which controls expression of the Cap proteins is spatially separated from the Cap coding sequence and does not control Cap proteins expression. The inducible promoter (for example, Tet-On promoter) controlling the expression of the Cap proteins is stronger than the native p40 promoter, resulting in increased Cap proteins expression as compared to the v1.0 system. The coding sequences and promoters for the Rep proteins are separated from the coding sequence and the inducible promoter for the Cap proteins by a transcription blocking element (TBE). This construct may also be referred to as a Rep-Cap construct.

FIG. 2B depicts the on state of the system of polynucleotides for inducibly producing rAAV depicted in FIG. 2A, after induction by a first triggering agent, e.g., doxycycline, and a second triggering agent, e.g., tamoxifen. The Cre coding element is positioned between LoxP sites and is additionally fused to estrogen response elements (“ER2”), which allows for control over the localization of Cre in response to estrogen agonists, such as tamoxifen. Upon addition of a first triggering agent, e.g., doxycycline, Cre is expressed and Cap proteins are expressed, and upon addition of the second triggering agent, e.g., tamoxifen, Cre translocates to the nucleus. Following translation and translocation of the Cre protein into the cell nucleus, the Cre protein effects excision of its own coding sequence, leaving the integrated Construct 2 as shown in FIG. 2B. Therefore, adenoviral E2A and E4 helper proteins are expressed. Cre also excises out the excisable element flanked by LoxP sites in Construct 1, allowing AAV rep coding sequences expression under control of native promoters. rAAV virions encapsidating the payload, such as a GOI, are therefore produced.

FIG. 3 shows an exemplary schematic for producing a rAAV from a cell.

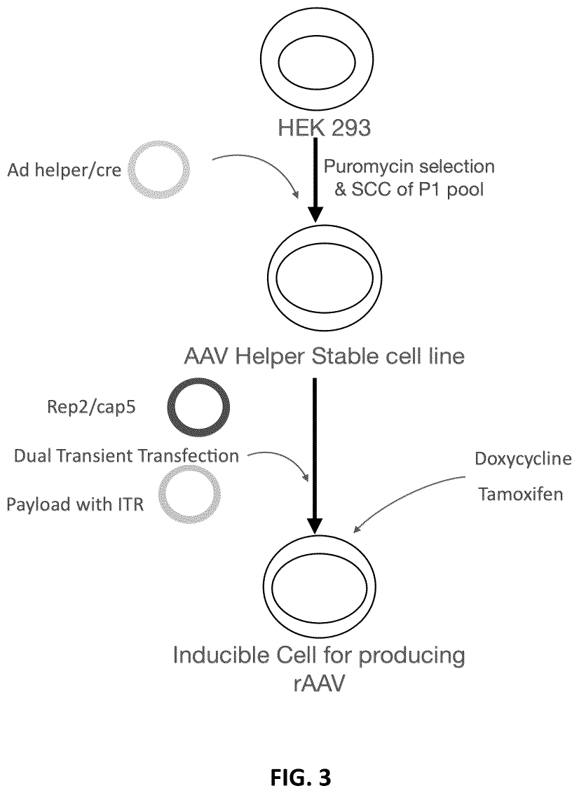

### DETAILED DESCRIPTION

Provided herein are various stable cell lines and methods of using the stable cell lines to produce recombinant AAV (rAAV) virions within which are packaged an expressible payload. In some embodiments, the stable cell line is a stable cell line that is transiently transfected with one or more plasmids, wherein these cells are capable of conditionally producing recombinant AAV (rAAV) virions within which are packaged an expressible payload; and production of virions is inducible upon addition of a triggering agent. In some embodiments, the stable cell line is a stable AAV helper cell line that is transiently transfected with a Rep/Cap plasmid and a payload plasmid. In some embodiments, the stable cell line is a stable Rep/Cap cell line that is transiently transfected with a AAV helper plasmid and a payload plasmid. In some embodiments, the stable cell line is a payload stable cell line that is transiently transfected with a Rep/Cap plasmid and an AAV helper plasmid. In some embodiments, the triggering agent is doxycycline. In some embodiments, the triggering agent is tamoxifen. In some embodiments, a first triggering agent and a second triggering agent induce virion production. In some embodiments, the first triggering agent is doxycycline and the second triggering agent is tamoxifen.

### Definitions

Unless defined otherwise, all technical and scientific terms used herein have the meaning commonly understood by one of ordinary skill in the art to which the invention pertains.

The term “about”, particularly in reference to a given quantity, is meant to encompass deviations of up to plus or minus five percent.

“AAV” is an abbreviation for adeno-associated virus and may be used to refer to the virus itself or derivatives thereof. The term covers all subtypes and both naturally occurring and recombinant forms, except where required otherwise. The components of the AAV DNA genome consist of two open reading frames, Rep and Cap, flanked by two 145 base inverted terminal repeats (ITRs). Rep gene encodes multiple distinct proteins including Rep78, Rep68, Rep52, and Rep40. These proteins are also referred to herein as Rep proteins or Rep and may encompass one or more of Rep78, Rep68, Rep52, and Rep40 and functional variants thereof and homologs thereof. Rep78 and Rep68 and functional variants thereof and homologs thereof are referred to herein as large Rep. Rep52, and Rep40 and functional variants thereof and homologs thereof are referred to herein as small Rep. Rep proteins from an AAV of a particular serotype may also be referred to as Rep1, Rep2, etc. where the Rep protein is derived from an AAV1 or an AAV2 serotype, respectively. Cap gene encodes capsid proteins VP1, VP2, and VP3 required for production of rAAV capsids. These proteins are also referred to herein as Cap proteins or Cap and may encompass one or more of VP1, VP2, and VP3 and functional variants thereof and homologs thereof. Cap proteins from an AAV of a particular serotype may also be referred to as Cap1, Cap2, Cap4, etc. where the Rep protein is derived from an AA1, an AAV2, or an AAV5 serotype, respectively. In addition to Rep and Cap, AAV requires a helper plasmid containing genes from a helper virus such as adenovirus, including E1a, E1b, E4, E2a, and VA genes for AAV replication.

“Recombinant”, as applied to an AAV virion, means that the rAAV virion (synonymously, rAAV virus particle) is the product of one or more procedures that result in an AAV particle Construct that is distinct from an AAV virion in nature. The procedure may be genetic alteration, e.g., by the addition or insertion of a heterologous nucleic acid Construct into the virus.

“Recombinant virus” is meant to describe a virus that has been genetically altered, e.g., by the addition or insertion of a heterologous nucleic acid construct into the virus.

The abbreviation “rAAV” refers to recombinant adeno-associated virus, also referred to as a recombinant AAV vector (or “rAAV vector”). The term “AAV” includes any AAV serotype as well as AAV vectors based on the combination of different serotypes (also referred to as “hybrid AAV vectors” or “pseudotype AAV vectors”). AAV serotype may be AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, AAV-7m8, AAV-6.2, AAV-Dj, AAV-DJ/8, AAV2-retro, AAV2-QuadYF and AAV2.7m8, AAV-PHP.B, AAV-PHP.B2, AAV-PHP.B3, AAV-PHP.A, AAV-PHP.eB, AAV-PHP.eS, evolved capsids that are less immunogenic to mice and humans, and variants thereof and combinations thereof. “Primate AAV” refers to AAV isolated from a primate, “non-primate AAV” refers to AAV isolated from a non-primate mammal, “bovine AAV” refers to AAV isolated from a bovine mammal (e.g., a cow), etc. An “rAAV vector” comprises a polynucleotide sequence not of AAV origin (i.e., a polynucleotide heterologous to AAV), typically a polynucleotide sequence of interest for introducing into a target cell. In general, the heterologous polynucleotide is flanked by at least one, and usually by two AAV inverted terminal repeat sequences (ITRs). The heterologous polynucleotide can also be referred to as a polynucleotide payload. The term rAAV vector encompasses both rAAV virions and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e., a polynucleotide other than a wild-type AAV genome, such as a transgene to be delivered to a mammalian cell), it is typically referred to as an “rAAV vector particle” or simply an “rAAV vector”. Thus, production of a rAAV particle necessarily includes production of a rAAV vector, as such a vector contained within an rAAV particle.

“Packaging” refers to a series of intracellular events that result in the assembly, encapsidation, and production of an AAV particle.

AAV “rep” and “cap” genes refer to polynucleotide sequences encoding replication and capsid proteins of adeno-associated virus. AAV rep and cap are referred to herein as AAV “packaging genes.”

By “AAV Rep coding region” or “sequence encoding one or more Rep proteins” or “Rep encoding sequence” and grammatical equivalents thereof is meant the art-recognized region of the AAV genome which encodes the replication proteins of the virus which are required to replicate the viral genome and/or a payload flanked by ITRs. The rep coding region, as used herein, may be derived from any viral serotype, such as those described above. The region need not include all of the wild-type genes but may be altered, e.g., by the insertion, deletion or substitution of nucleotides, so long as the rep genes provide for expression Rep proteins. Rep coding sequences are further described below. The terms “AAV Rep coding sequence” and “AAV Rep proteins coding sequence” are used interchangeably herein. The terms “AAV Rep proteins”, “Rep proteins”, “AAV Rep polypeptide”, and “Rep polypeptide” are interchangeably used herein.

By “AAV cap coding region” or “sequence encoding one or more cap proteins,” or “Cap encoding sequence” and grammatical equivalents thereof it is meant the art-recognized region of the AAV genome which encodes the coat proteins of the virus which are required for the capsid that viral genome or a payload is packaged into by the Rep proteins. For a further description of the cap coding region, see, e.g., Muzyczka, N. (1992) Current Topics in Microbiol. and Immunol. 158, 97-129; Kotin, R. M. (1994) Human Gene Therapy 5, 793-801. The AAV cap coding region, as used herein, may be derived from any AAV serotype, as described above. The region need not include all of the wild-type cap genes but may be altered, e.g., by the insertion, deletion or substitution of nucleotides, so long as the genes provide for sufficient packaging functions. Cap coding sequences are further described below. The term “AAV Cap coding sequence” and “AAV Cap proteins coding sequence” are interchangeably used herein. The terms “AAV Cap proteins”, “Cap proteins”, “AAV Cap polypeptide”, and “Cap polypeptide” are interchangeably used herein. The term “Capsid” and “Cap” are interchangeably used herein.

By “adeno-associated virus inverted terminal repeats” or “AAV ITRs” is meant the art-recognized regions found at each end of the AAV genome which function together in cis as origins of DNA replication and as packaging signals for the viral genome. The nucleotide sequences of AAV ITR regions are known. See, e.g., Kotin, R. M. (1994) Human Gene Therapy 5, 793-801; Berns, K. I. “Parvoviridae and their Replication” in Fundamental Virology, 2d ed., (B. N. Fields and D. M. Knipe, eds.) for the AAV-2 ITRs sequence. As used herein, an “AAV ITR” need not have a wild-type nucleotide sequence, but may be altered, e.g., by the insertion, deletion or substitution of nucleotides. The AAV ITR may be derived from any of several AAV serotypes, including without limitation, AAV-1, AAV-2, AAV-3, AAV-4, AAV-5, AAV-7, etc. Furthermore, 5′ and 3′ ITRs which flank a selected nucleotide sequence in an AAV vector need not necessarily be identical or derived from the same AAV serotype or isolate. The ITRs may be single stranded (ssITRs) or self-complementary (scITRs).

A “helper virus” for AAV refers to a virus that allows AAV (e.g., wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

The term “adenoviral helper proteins” and “AAV helper proteins” are interchangeably used herein.

“Helper virus function(s)” refers to function(s) encoded in a helper virus genome which allows AAV replication and packaging (in conjunction with other requirements for replication and packaging described herein). As described herein, “helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component which the particle is capable of delivering into a cell for which the viral species is tropic. The term does not necessarily imply any replication capacity of the virus. As used herein, an “infectious” virus or viral particle is one that may access a target cell, may infect a target cell, and may express a heterologous nucleic acid in a target cell. Thus, “infectivity” refers to the ability of a viral particle to access a target cell, infect a target cell, and express a heterologous nucleic acid in a target cell. Infectivity may refer to in vitro infectivity or in vivo infectivity. Assays for counting infectious viral particles are described elsewhere in this disclosure and in the art. Viral infectivity may be expressed as the ratio of infectious viral particles to total viral particles. Total viral particles may be expressed as the number of viral genome (vg) copies. The ability of a viral particle to express a heterologous nucleic acid in a cell may be referred to as “transduction.” The ability of a viral particle to express a heterologous nucleic acid in a cell may be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity may be expressed as the ratio of infectious viral particles to total viral particles. Methods of determining the ratio of infectious viral particle to total viral particle are known in the art. See, e.g., Grainger et al. (2005) Mol. Ther. 11:5337 (describing a TCID50 infectious titer assay); and Zolotukhin et al. (1999) Gene Ther. 6:973.

The term “polynucleotide” refers to a polymeric form of nucleotides of any length, including deoxyribonucleotides or ribonucleotides, or analogs thereof. The terms “polynucleotide” and “nucleic acid” are interchangeably used herein. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

As used herein, the term “polynucleotide construct” refers to a DNA segment of any size that includes one or more sequences encoding an RNA or protein and at least one promoter for driving expression from the one or more sequences. The term “polynucleotide construct” and “nucleic acid construct” are interchangeably used herein. A polynucleotide construct may be a circular DNA or a linear DNA. A polynucleotide construct may be single stranded or double stranded. As used herein, the term “vector” includes any genetic element, such as a plasmid, phage, transposon, cosmid, chromosome, artificial chromosome, virus, virion, etc., which is capable of replication when associated with the proper control elements and which may transfer gene sequences into and between cells. Thus, the term includes cloning and expression vehicles, as well as viral vectors. The use of the term “vector” throughout this specification encompasses plasmid or viral vectors, which permit the desired components to be transferred to the host cell via transfection or infection. For example, an adeno-associated viral (AAV) vector is a plasmid comprising a recombinant AAV genome. In some embodiments, useful vectors are contemplated to be those vectors in which the nucleic acid segment to be transcribed is positioned under the transcriptional control of a promoter. A vector may be linear or circular, single stranded or double stranded, DNA or RNA. In certain aspects, the vector may be circular, double stranded DNA.

As used herein, the term “vector system” refers to two or more vectors that are used together, e.g., by simultaneous or sequential introduction into a cell, to provide at least two different components into the cell. The two different components may then work together in the cell.

A polynucleotide or polypeptide has a certain percent “sequence identity” to another polynucleotide or polypeptide, meaning that, when aligned, that percentage of bases or amino acids are the same when comparing the two sequences. The term percent “sequence identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “sequence identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows:

$$Percent Sequence Identity = ( “ Percent Identity ” output value ) \times ( “ Query Coverage ” output value )$$

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023 are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows:

$$( Number of Identical Positions ) / \\ ( Total Number of Positions in the Test Sequence ) ] \times 100 \%$$

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (SEQ ID NO: 116; length=10 nucleotides) to reference sequence 2: AAAAAAAAAA (SEQ ID NO: 117; length=10 nucleotides). The percent identity between test sequence 1 and reference sequence 2 would be [(5)1(10)]×100%=50%. Test sequence 1 has 50% sequence identity to reference sequence 2. In another example, percent identity is calculated to compare test sequence 3: CCCCCGGGGGGGGGGCCCCC (SEQ ID NO: 118; length=20 nucleotides) to reference sequence 4: GGGGGGGGGG (SEQ ID NO: 119; length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (SEQ ID NO: 119; length=10 nucleotides) to reference sequence 6: CCCCCGGGGGGGGGGCCCCC (SEQ ID NO: 118; length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (SEQ ID NO: 120; length=10 amino acids) to reference sequence 8: YYYYYYYYYY (SEQ ID NO: 121; length=10 amino acids). The percent identity between test sequence 7 and reference sequence 8 would be [(5)1(10)]×100%=50%. Test sequence 7 has 50% sequence identity to reference sequence 8. In another example, percent identity is calculated to compare test sequence 9: LLLLLFFFFFYYYYYLLLLL (SEQ ID NO: 122; length=20 amino acids) to reference sequence 10: FFFFFYYYYY (SEQ ID NO: 120; length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (SEQ ID NO: 120; length=10 amino acids) to reference sequence 12: LLLLLFFFFFYYYYYLLLLL (SEQ ID NO: 122; length=20 amino acids). The percent identity between test sequence 11 and reference sequence 12 would be [(10)/(10)]×100%=100%. Test sequence 11 has 100% sequence identity to reference sequence 12.

For purposes herein, reference to a polynucleotide sequence (e.g., a DNA sequence or an RNA sequence) also encompasses the reverse complement of the polynucleotide sequence. For example, a sequence of AAAAAGGGGG (SEQ ID NO: 116) also encompasses a sequence of CCCCCTTTTT (SEQ ID NO: 123).

A “gene” refers to a polynucleotide containing at least one open reading frame that is capable of encoding a particular protein after being transcribed and translated.

The term “host cell” denotes, for example, microorganisms, yeast cells, insect cells, and mammalian cells, that may be, or have been, used as recipients of an AAV vector system as described herein, or other transfer DNA. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement to the original parent, due to natural, accidental, or deliberate mutation. In some aspects, the disclosure provides transfected host cells.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

As used herein, the term “cell line” refers to a population of cells capable of continuous or prolonged growth and division in vitro. Often, cell lines are clonal populations derived from a single progenitor cell. It is further known in the art that spontaneous or induced changes may occur in karyotype during storage or transfer of such clonal populations. Therefore, cells derived from the cell line referred to may not be precisely identical to the ancestral cells or cultures, and the cell line referred to includes such variants.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “intermediate cell line” refers to a cell line that contains the AAV rep and cap components integrated into the host cell genome or a cell line that contains the adenoviral helper functions integrated into the host cell genome.

As used herein, the term “packaging cell line” refers to a cell line that contains the AAV rep and cap components and the adenoviral helper functions integrated into the host cell genome. A payload construct must be added to the packaging cell line to generate rAAV virions.

As used herein, the term “production cell line” refers to a cell line that contains the AAV rep and cap components, the adenoviral helper functions, and a payload construct. The rep and cap components and the adenoviral helper functions are integrated into the host cell genome. The payload construct can be stably integrated into the host cell genome or transiently transfected. rAAV virions can be generated from the production cell line upon the introduction of one or more triggering agents in the absence of any plasmid or transfection agent.

As used herein, the term “downstream purification” refers to the process of separating rAAV virions from cellular and other impurities. Downstream purification processes include chromatography-based purification processes, such as ion exchange (IEX) chromatography and affinity chromatography.

The term “prepurification yield” refers to the rAAV yield prior to the downstream purification processes. The term “postpurification yield” refers to the rAAV yield after the downstream purification processes. rAAV yield can be measured as viral genome (vg)/L.

The encapsidation ratio of a population of rAAV virions can be measured as the ratio of rAAV viral particle (VP) to viral genome (VG). The rAAV viral particle includes empty capsids, partially full capsids (e.g., comprising a partial viral genome), and full capsids (e.g., comprising a full viral genome).

The F:E ratio of a population of rAAV virions can be measured as the ratio of rAAV full capsids to empty capsids. The rAAV full capsid particle includes partially full capsids (e.g., comprising a partial viral genome) and full capsids (e.g., comprising a full viral genome). The empty capsids lack a viral genome.

The potency or infectivity of a population of rAAV virions can be measured as the percentage of target cells infected by the rAAV virions at a multiplicity of infection (MOI; viral genomes/target cell). Exemplary MOI values are 1×10¹, 1×10², 2×10³, 5×10⁴, or 1×10⁵ vg/target cell. An MOI can be a value chosen from the range of 1×101 to 1×10⁵ vg/target cell.

The term “expression vector or construct” or “synthetic construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The term “auxotrophic” or “auxotrophic selectable marker” as used herein refers to the usage of a medium lacking a supplement, such as a medium lacking an essential nutrient such as the purine precursors hypoxanthine and thymidine (HT), or the like, for selection of a functional enzyme which allows for growth in the medium lacking the essential nutrient, e.g., a functional dihydrofolate reductase or the like.

The terms “tetracycline” is used generically herein to refer to all antibiotics that are structurally and functionally related to tetracycline, including tetracycline, doxycycline, demeclocycline, minocycline, sarecycline, oxytetracycline, omadacycline, or eravacycline.

The terms “constitutive” or “constitutive expression” are used interchangeably herein. They refer to genes that are transcribed in an ongoing manner. Such gene are driven by a constitutive promoter. In some embodiments, the terms refer to the expression of a therapeutic payload or a nucleic acid sequence that is not conditioned on addition of an triggering agent to the cell culture medium. A constitutive promoter is capable of directing continuous gene expression in a cell. Constitutive promoters regulate expression of basal genes, like housekeeping genes. In contrast, an inducible promoter directs gene expression in the presence of an external stimulus. Thus, an inducible promoter can be controlled by providing or withdrawing the stimulus.

As used herein, the term “polynucleotide payload” refers to a polynucleotide sequence that is packaged into a rAAV virion for delivery by the rAAV virion into a cell. A polynucleotide payload is flanked by AAV inverted terminal repeats (ITRs). Upon delivery to a cell, the polynucleotide payload may be available to the cell as a DNA (e.g., a homology region for homology-directed repair), transcribed into an RNA (e.g., a guide RNA (gRNA), a tRNA, a suppressor tRNA, a siRNA, a miRNA, an mRNA, a shRNA, a circular RNA, an antisense oligonucleotide (ASO)), or transcribed and translated into a polypeptide (e.g., an antibody, a hormone, a site-specific endonuclease, a reporter gene, a component of a CRISPR/Cas system, an adenosine deaminase acting on RNA (ADAR) enzyme, a transcriptional activator, a transcriptional repressor, a ribozyme, or a DNAzyme.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter. A promoter is usually upstream of a gene whose expression is controlled by the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. Additional sequences or sequence motifs operably linked to a sequence where it is not naturally found are also heterologous; such sequences or sequence motifs include polyA signal sequences, introns, and/or any other relevant sequence. Thus, for example, an rAAV that includes a heterologous nucleic acid encoding a heterologous payload is an rAAV that includes a nucleic acid not normally included in a naturally occurring, wild-type AAV, and the encoded heterologous payload is a payload not normally encoded by a naturally-occurring, wild-type AAV. As another example, a Capsid proteins coding sequence operably linked to a heterologous polyA signal sequence refers to a AAV Cap coding sequence operably linked to a sequence not native to AAV.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell. For example, a gene integrated into the nuclear genome of the cell and is available to perform its function during extended culture of the cell in vitro. A gene integrated into the nuclear genome of the cell is inheritable by progeny of the cell.

A cell is said to be “stably” altered, transduced, genetically modified, or transformed with a genetic sequence if the sequence is available to perform its function during extended culture of the cell in vitro. Generally, such a cell is “heritably” altered (genetically modified) in that a genetic alteration is introduced which is also inheritable by progeny of the altered cell. For example, a gene integrated into the nuclear genome of the cell and is available to perform its function during extended culture of the cell in vitro. A gene integrated into the nuclear genome of the cell is inheritable by progeny of the cell.

The terms “polypeptide,” “peptide,” and “protein” are used interchangeably herein to refer to polymers of amino acids of any length. The terms also encompass an amino acid polymer that has been modified; for example, disulfide bond formation, glycosylation, lipidation, phosphorylation, or conjugation with a labeling component. Polypeptides such as anti-angiogenic polypeptides, neuroprotective polypeptides, and the like, when discussed in the context of delivering a payload to a mammalian subject, and compositions therefor, refer to the respective intact polypeptide, or any fragment or genetically engineered derivative thereof, which retains the desired biochemical function of the intact protein. Similarly, references to nucleic acids encoding anti-angiogenic polypeptides, nucleic acids encoding neuroprotective polypeptides, and other such nucleic acids for use in delivery of a payload to a mammalian subject (which may be referred to as “transgenes” to be delivered to a recipient cell), include polynucleotides encoding the intact polypeptide or any fragment or genetically engineered derivative possessing the desired biochemical function.

An “isolated” plasmid, nucleic acid, vector, virus, virion, host cell, or other substance refers to a preparation of the substance devoid of at least some of the other components that may also be present where the substance or a similar substance naturally occurs or is initially prepared from. Thus, for example, an isolated substance may be prepared by using a purification technique to enrich it from a source mixture. Enrichment may be measured on an absolute basis, such as weight per volume of solution, or it may be measured in relation to a second, potentially interfering substance present in the source mixture. Increasing enrichments of the embodiments of this invention are increasingly more isolated. An isolated plasmid, nucleic acid, vector, virus, host cell, or other substance is in some cases purified, e.g., from about 80% to about 90% pure, at least about 90% pure, at least about 95% pure, at least about 98% pure, or at least about 99%, or more, pure.

The terms “treatment”, “treating”, “treat” and the like are used herein to generally refer to obtaining a desired pharmacologic and/or physiologic effect. The effect may be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment may include those already afflicted (e.g., those with a neurological disorder) as well as those in which prevention is desired (e.g., those with increased susceptibility to a neurological disorder; those suspected of having a neurological disorder; those having one or more risk factors for a neurological disorder, etc.).

A “therapeutically effective amount” or “efficacious amount” means the amount of a compound that, when administered to a mammal or other subject for treating a disease, is sufficient, in combination with another agent, or alone in one or more doses, to effect such treatment for the disease. The “therapeutically effective amount” will vary depending on the compound, the disease and its severity and the age, weight, etc., of the subject to be treated.

The terms “individual,” “host,” “subject,” and “patient” are used interchangeably herein, and refer to a mammal, including, but not limited to, human and non-human primates, including simians and humans; mammalian sport animals (e.g., horses, camels, etc.); mammalian farm animals (e.g., sheep, goats, cows, etc.); mammalian pets (dogs, cats, etc.); and rodents (e.g., mice, rats, etc.). In some cases, the individual is a human.

The terms “hybridize” and “hybridization” refer to the formation of complexes between nucleotide sequences which are sufficiently complementary to form complexes via Watson Crick base pairing.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule may have two homologous regions which are capable of hybridizing to each other. Thus, the term “homologous region” includes nucleic acid segments with complementary sequences. Homologous regions may vary in length but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands may base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine.

However, when a uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

As used herein, the term “recombination site” denotes a region of a nucleic acid molecule comprising a binding site or sequence-specific motif recognized by a site-specific recombinase that binds at the target site and catalyzes recombination of specific sequences of DNA at the target site. Site-specific recombinases catalyze recombination between two such target sites. The relative orientation of the target sites determines the outcome of recombination. For example, translocation occurs if the recombination sites are on separate DNA molecules. DNA between two recombination sites oriented in the same direction on the same DNA molecule will be excised as a circular loop of DNA. DNA between two recombination sites that are orientated in the opposite direction on the same DNA molecule will be inverted.

### Overview

Disclosed herein are polynucleotide constructs that are integrated into the nuclear genome of a cell to produce a stable cell line that is capable of conditionally producing recombinant AAV (rAAV) virions within which are packaged an expressible payload. Disclosed herein are stable cell lines capable of conditionally producing recombinant AAV (rAAV) virions within which are packaged an expressible payload. The stable cell line may be a stable AAV helper cell line, wherein an AAV helper construct is integrated into the genome of the cell. The stable cell line may be a stable Rep/Cap cell line, wherein a Rep/Cap construct is integrated into the genome of the cell. The stable cell line may be a payload stable cell line, wherein a payload construct is integrated into the genome of the cell. The stable cell line may further comprise one or more polynucleotide constructs, wherein the one or more polynucleotide constructs may not be integrated into the genome, e.g., may be present in plasmids, for conditionally producing recombinant AAV (rAAV) virions within which are packaged an expressible payload. In some embodiments, the nucleic acid molecules encoding the other elements needed for producing rAAV are introduced into the stable cell line via transfection, such as in plasmids via transient transfection. In some embodiments, the cell is a mammalian cell; optionally, wherein the mammalian cell is a HEK293 cell.

### Polynucleotides for AAV Production

### First Polynucleotide Encoding AAV Rep and Cap Proteins

The first polynucleotide may include a sequence encoding AAV Rep proteins operably linked to one or more promoters. In some embodiments, the one or more promoters may be native AAV promoters. In some embodiments, the native AAV promoters comprise P5 and P19 AAV promoters.

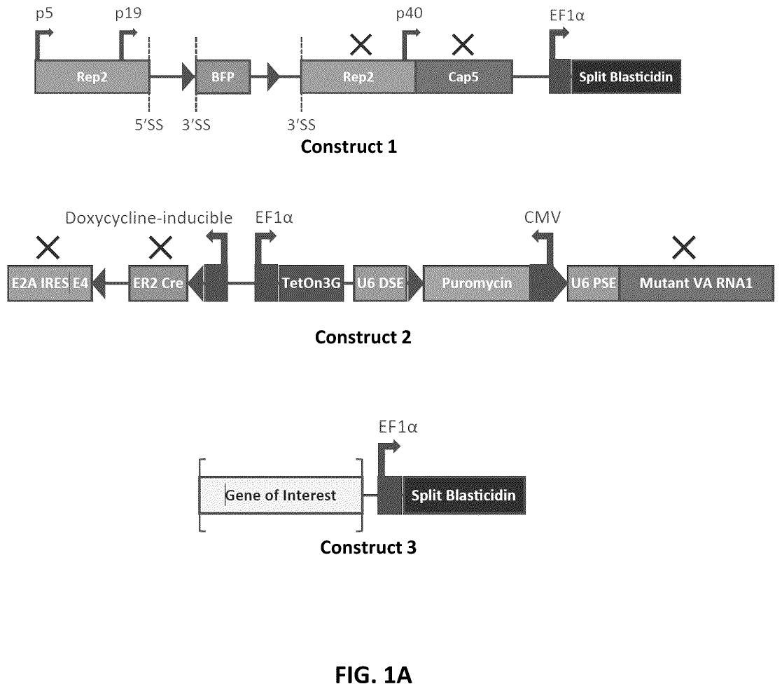

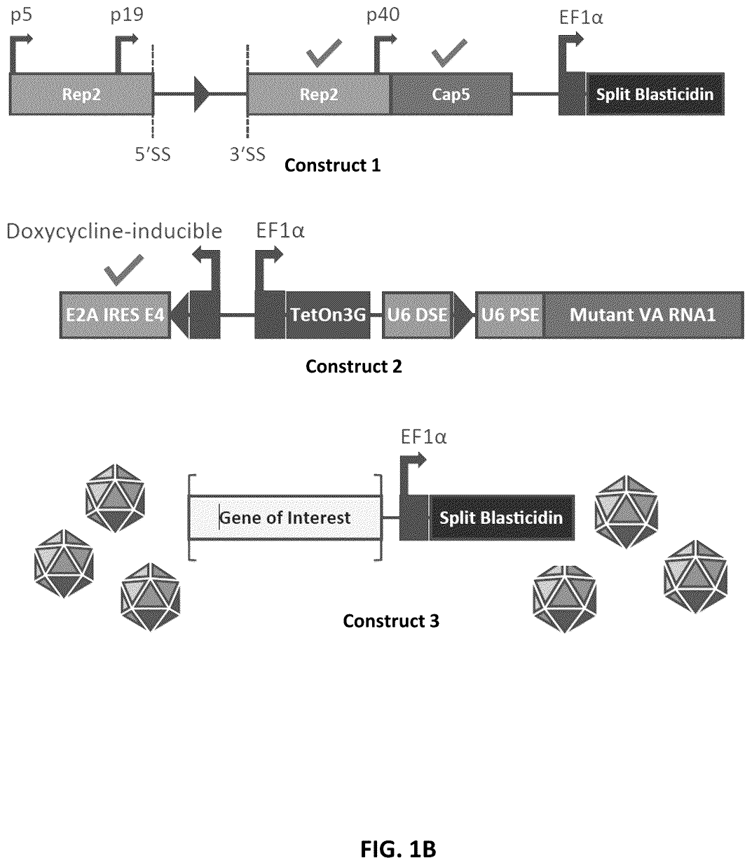

Turning to FIG. 1A, in some embodiments, the first polynucleotide comprises a sequence encoding AAV Rep proteins operably linked to one or more promoters comprises from 5′ to 3′: one or more promoters operably linked to a first sequence comprising a first part of the AAV Rep coding sequence; a second sequence comprising a second part of the AAV Rep coding sequence, wherein the one or more promoters are not operably linked to the second sequence comprising the second part of the AAV Rep coding sequence, wherein the first sequence and the second sequence are separated by (i) an excisable element that comprises a first recombination site and a second recombination site flanking a coding sequence encoding a stop signaling sequence, wherein the first recombination site and the second recombination site are oriented in the same direction or (ii) an inversible element that comprises a first recombination site and a second recombination site flanking a coding sequence encoding a stop signaling sequence, wherein the first recombination site and the second recombination site are oriented in opposite directions; and a third sequence comprising the sequence encoding one or more AAV capsid proteins, wherein the second sequence comprises a promoter that is operably linked to the third sequence; and the first constitutive promoter operably linked to a sequence encoding the first selectable marker.

In some embodiments, the first polynucleotide comprising a sequence encoding AAV Rep proteins operably linked to one or more promoters comprises from 5′ to 3′: the one or more promoters operably linked to a first sequence comprising a first part of an AAV Rep coding sequence, a 5′ splice site, a first part of an intron, a first recombination site, a first 3′ splice site, a coding sequence comprising a stop signaling sequence, a second recombination site, a second part of the intron, a second 3′ splice site, and a second sequence comprising a second part of the AAV Rep coding sequence. In certain embodiments, the first recombination site, the first 3′ splice site, the coding sequence comprising the stop signaling sequence, and the second recombination site form an excisable element. In certain embodiments, the first recombination site and the second recombination site are oriented in the same direction. In certain embodiments, the one or more promoters are not operably linked to the second sequence comprising the second part of the AAV Rep coding sequence. In certain embodiments, the first and second recombination sites are recombined by the inducible recombinase in the presence of a first triggering agent and a second triggering agent resulting in excision of the excisable element.

In certain embodiments, the first part of the AAV Rep coding sequence and the first part of the intron are joined to the second part of the intron and the second part of the AAV Rep coding sequence to form a complete AAV Rep coding sequence, allowing expression of AAV Rep proteins.

In some embodiments, the coding sequence encoding the stop signaling sequence of the first polynucleotide encodes for from 5′ to 3′: an exon and the stop signaling sequence. In some embodiments, the coding sequence encoding the stop signaling sequence of the first polynucleotide further comprises a sequence encoding a protein marker. In certain embodiments, the sequence encoding the protein marker is in-frame with the stop signaling sequence.

In some embodiments, the first polynucleotide further comprises a sequence encoding AAV Cap proteins. In some embodiments, transcription of the sequence encoding the AAV Cap proteins on the first polynucleotide is driven by a native promoter. In certain embodiments, the native promoter is a P40 AAV promoter. In some embodiments, the Rep coding sequence is 5′ to the Cap coding sequence. In certain embodiments, the Cap coding sequence is operatively linked to an endogenous P40 promoter present within the Rep proteins coding sequence.

In some embodiments, the first polynucleotide has at least 5%, 10%, 20%, 30%, 40%, 50%, 60%, 70%, 75%, 80%, 85%, 90%, 95%, or 100% sequence identity to SEQ ID NO: 1; SEQ ID NO: 2; SEQ ID NO: 3; SEQ ID NO: 4; SEQ ID NO: 24; SEQ ID NO: 99; or SEQ ID NO: 104.

In some embodiments, the first polynucleotide has at least 70%, at least 75%, at least 80%, at least 85%, at least 90%, at least 95%, at least 97%, at least 98%, at least 99%, or 100% sequence identity to SEQ ID NO: 24. In certain embodiments, the first polynucleotide has sequence of SEQ ID NO: 24.

In some embodiments, the first polynucleotide has at least 70%, at least 75%, at least 80%, at least 85%, at least 90%, at least 95%, at least 97%, at least 98%, at least 99%, or 100% sequence identity to SEQ ID NO: 24 but lacks the sequence of SEQ ID NO: 25 downstream of the sequence corresponding to the Cap sequence of SEQ ID NO: 24. In certain embodiments, the first polynucleotide has sequence of SEQ ID NO: 24 but lacks the sequence of SEQ ID NO: 25 downstream of the sequence corresponding to the Cap sequence of SEQ ID NO: 24.

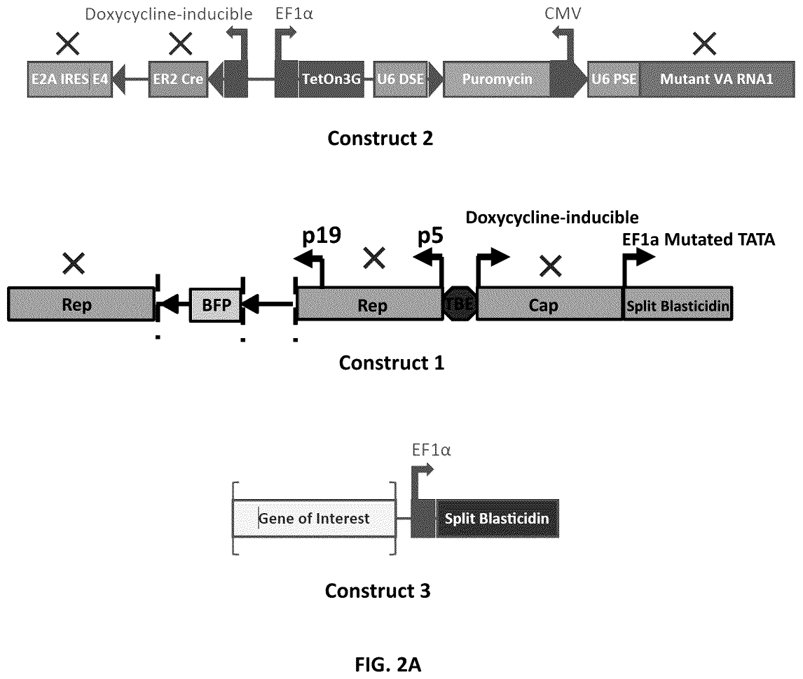

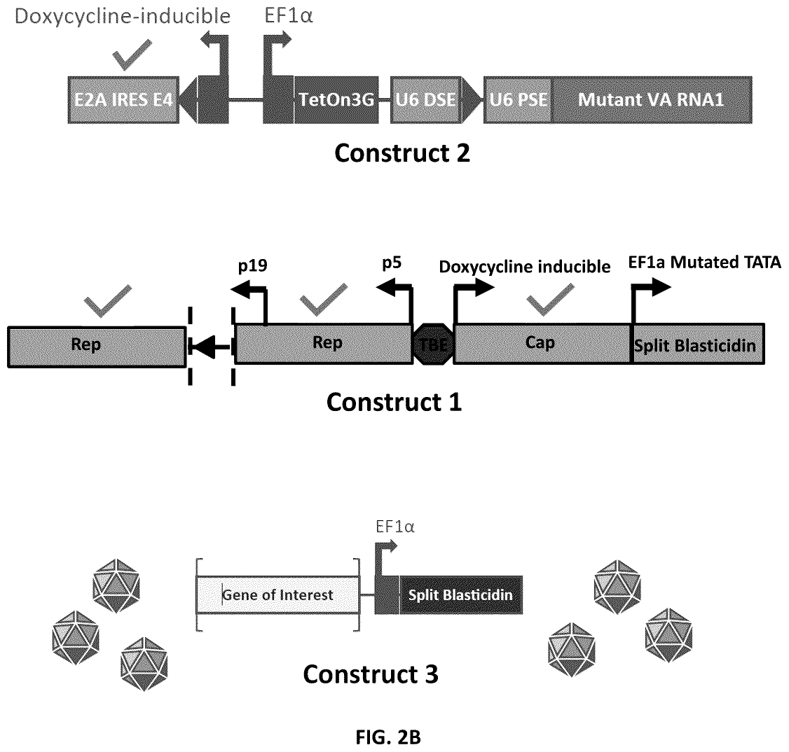

FIG. 2A illustrate an alternative arrangement of the first polynucleotide, where the first sequence encoding AAV Rep proteins is separated from the second sequence by an intervening sequence. In some embodiments, the intervening sequence comprises a transcriptional blocking element (TBE). In other embodiments, optionally, a sequence of the TBE has at least 70%, at least 75%, at least 80%, at least 85%, at least 90%, at least 95%, at least 97%, at least 98%, at least 99%, or 100% sequence identity to SEQ ID NO: 103. In certain embodiments, a sequence of the TBE has sequence of SEQ ID NO: 103.

In some embodiments, the first polynucleotide comprises an AAV Rep coding sequence, a sequence encoding one or more AAV capsid proteins, and a first constitutive promoter operably linked to a sequence encoding a first selectable marker. In some embodiments, the first polynucleotide comprises: (i) from 5′ to 3′: (A) one or more promoters operably linked to a first sequence comprising a first part of the AAV Rep coding sequence; a second sequence comprising a second part of the AAV Rep coding sequence, wherein the one or more promoters are not operably linked to the second sequence comprising the second part of the AAV Rep coding sequence, wherein the first sequence and the second sequence are separated by (I) an excisable element that comprises a first recombination site and a second recombination site flanking a coding sequence encoding a stop signaling sequence, wherein the first recombination site and the second recombination site are oriented in the same direction or (II) an inversible element that comprises a first recombination site and a second recombination site flanking a coding sequence encoding a stop signaling sequence, wherein the first recombination site and the second recombination site are oriented in opposite directions; or (B) one or more promoters operably linked to a sequence comprising a first part of an AAV Rep coding sequence, a 5′ splice site, a first part of an intron, a first recombination site, a first 3′ splice site, a coding sequence comprising a stop signaling sequence, a second recombination site, a second part of the intron, a second 3′ splice site, and a sequence comprising a second part of the AAV Rep coding sequence, wherein the first recombination site, the first 3′ splice site, the coding sequence comprising the stop signaling sequence, and the second recombination site form an excisable element, wherein the first recombination site and the second recombination site are oriented in the same direction, and wherein the one or more promoters are not operably linked to the sequence comprising the second part of the AAV Rep coding sequence, wherein the first and second recombination sites are recombined by the inducible recombinase in the presence of a first triggering agent and a second triggering agent resulting in excision of the excisable element, and the first part of the AAV Rep coding sequence and the first part of the intron are joined to the second part of the intron and the second part of the AAV Rep coding sequence to form a complete AAV Rep coding sequence, allowing expression of AAV Rep proteins; (ii) a third sequence comprising the sequence encoding the one or more AAV capsid proteins operably linked to a first inducible promoter; optionally, further comprising a polyadenylation signal sequence, wherein the polyadenylation signal sequence encodes a stronger polyadenylation signal than a native AAV Cap polyadenylation signal sequence and is a 3′ of the sequence encoding the one or more AAV capsid proteins; and (iii) the first constitutive promoter operably linked to a sequence encoding the first selectable marker.

In some embodiments, the first polynucleotide comprises at least 5%, 10%, 20%, 30%, 40%, 50%, 60%, 70%, 75%, 80%, 85%, 90%, 95%, or 100% sequence identity to SEQ ID NO: 101; SEQ ID NO: 104; SEQ ID NO: 114, SEQ ID NO: 115; or SEQ ID NO: 114 and SEQ ID NO: 115.

In some embodiments, the first polynucleotide further comprises a sequence encoding AAV Cap proteins. In some embodiments, transcription of the sequence encoding the AAV Cap proteins on the first polynucleotide is driven by a native promoter. In certain embodiments, the native promoter is a P40 AAV promoter. In certain embodiments, transcription of the sequence encoding the AAV Cap proteins on the first polynucleotide is driven by a heterologous promoter. Heterologous promoters can include inducible promoters. Inducible promoters allow induction of transcription through the introduction of a triggering agent. In some embodiments, the inducible promoter comprises a tetracycline-inducible promoter or a cumate-inducible promoter. In some instances, the inducible promoter is a doxycycline-inducible promoter. In certain instances, the triggering agent is doxycycline.

In various embodiments (e.g., the embodiments illustrated in either FIG. 1A or FIG. 2A), the Rep coding sequence can encode Rep proteins from any desired AAV serotype. In some embodiments, the encoded Rep proteins are from the same serotype as the Cap proteins. In some embodiments, the encoded Rep proteins are from a different serotype from the Cap proteins. In particular embodiments, the encoded Rep proteins include, but are not limited to, Rep proteins from AAV serotypes AAV-1, AAV-2, AAV-3, AAV-4, AAV-5, AAV-6, AAV-7, AAV-8, AAV-9, AAV-10 and AAV-11, or chimeric combinations thereof.

The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol, 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al. Virol, 78: 6381-6388 (2004); the AAV-10 genome is provided in Mol Ther, 13(1): 67-76 (2006); and the AAV-11 genome is provided in Virology, 330(2): 375-383 (2004).

In some embodiments, the AAV capsid proteins comprise VP1, VP2, and VP3.

As explained herein, prior to the cell being contacted to the first triggering agent and the second triggering agent, the Rep coding sequence is interrupted by an excisable element. Addition of both the first triggering agent and the second triggering agent are required for excision of the excisable element. FIG. 1B and transcription of the sequence encoding the AAV Cap proteins on the first polynucleotide is driven by a native promoter illustrate schematics of the first polynucleotide after addition of the first triggering agent and the second triggering agent, which permits expression of the Rep proteins and the Cap proteins. In some embodiments, the excisable element is inserted at CAG-G, CAG-A, AAG-G, AAG-A, wherein the dash (-) indicates the point of insertion of the excisable element, in the Rep coding sequence, and the excisable element is inserted downstream (or 3′) of the p19 promoter. In some embodiments, the excisable element is inserted at CAG-G, CAG-A, AAG-G, AAG-A, wherein the dash (-) indicates the point of insertion of the excisable element, in the Rep coding sequence, and the excisable element is inserted downstream of the p19 promoter and upstream of the p40 promoter.

In certain embodiments, the excisable element comprises, from 5′ to 3′, a first spacer segment, a second spacer segment, and a third spacer segment.

In particular embodiments, the first spacer segment comprises a 5′ splice site (5′SS) 5′ to the first spacer element. In some embodiments, the first spacer segment comprises a nucleic acid sequence having at least 80% identity to SEQ ID NO: 26.

In some embodiments, the second spacer segment comprises a polynucleotide encoding a detectable protein marker flanked by lox sites. In certain embodiments, the detectable protein marker is a fluorescent protein. In particular embodiments, the fluorescent protein is a green or blue fluorescent protein (GFP of BFP). In specific embodiments, the GFP is EGFP. In particular embodiments, the fluorescent protein is a blue fluorescent protein (BFP). Screening for the fluorescent marker can be used to confirm integration of the construct into the cell genome and can subsequently be used to confirm excision of the intervening spacer segment. In some embodiments, the second spacer segment further comprises a polyA signal sequence. In some embodiments, the second spacer segment further comprises a first 3′ splice site (3′SS) between the first lox site and the polynucleotide encoding the protein marker.

In some embodiments, a stronger polyadenylation signal enhances RNA processing, RNA stability, RNA translation efficiency, or any combination thereof. In certain embodiments, the poly A signal sequence comprises SV40 polyadenylation signal sequence, a bovine growth hormone polyadenylation signal sequence, or a Rabbit Beta Globin polyadenylation signal sequence.

In some embodiments, the polyadenylation signal sequence has at least 70%, at least 75%, at least 80%, at least 85%, at least 90%, at least 95%, at least 97%, at least 98%, at least 99%, or 100% sequence identity to SEQ ID NO: 111, has at least 70%, at least 75%, at least 80%, at least 85%, at least 90%, at least 95%, at least 97%, at least 98%, at least 99%, or 100% sequence identity to SEQ ID NO: 110, or has at least 70%, at least 75%, at least 80%, at least 85%, at least 90%, at least 95%, at least 97%, at least 98%, at least 99%, or 100% sequence identity to SEQ ID NO: 113.

In some embodiments, the native AAV Cap polyadenylation signal sequence has at least 70%, at least 75%, at least 80%, at least 85%, at least 90%, at least 95%, at least 97%, at least 98%, at least 99%, or 100% sequence identity to SEQ ID NO: 112.

In some embodiments, the second spacer segment comprises a nucleic acid sequence having at least 80% identity to SEQ ID NO: 27.

In some embodiments, the third spacer segment further comprises a second 3′ splice site (3′SS). In particular embodiments, the second 3′ splice site is positioned 3′ to the second lox site.

In some embodiments, the third spacer segment comprises a nucleic acid sequence having at least 80% identity to SEQ ID NO: 28.

In various embodiments, the Rep coding sequences are operatively linked to an endogenous P5 promoter. In various embodiments, the Rep coding sequences are operatively linked to an endogenous P19 promoter. In various embodiments, the large Rep coding sequences are operatively linked to an endogenous P5 promoter and the small Rep coding sequences are operatively linked to an endogenous P19 promoter.

In some embodiments, the polynucleotide encoding the Rep coding sequences further comprises a constitutive promoter operably linked to a selectable marker. In some embodiments, the constitutive promoter is EF1alpha promoter or human cytomegalovirus promoter. In some embodiments, the EF1alpha promoter is a mutated EF1alpha promoter, such as an EF1alpha promoter comprising a mutation in a TATA box.In some embodiments, transcription of full-length AAV Rep coding sequences occurs after excision of the excisable element or inversion of the inversible element.

In some embodiments, the first polynucleotide is not flanked by inverted terminal repeat sequences.

### Second Polynucleotide Encoding Helper Proteins

Returning to polynucleotides of FIG. 1A, in some embodiments, the second polynucleotide comprises a constitutive promoter operably linked to a sequence encoding an activator. In some embodiments, the activator is unable to activate an inducible promoter in absence of a first triggering agent. In some embodiments, the second polynucleotide comprises from 5′ to 3′: (i) an inducible promoter operably linked to a self-excising element; and (ii) a sequence encoding one or more AAV helper proteins. In some embodiments, the second polynucleotide comprises a third constitutive promoter operably linked to a sequence encoding a second selectable marker. In some embodiments, the self-excising element comprising a third recombination site and a fourth recombination site flanking a sequence encoding an inducible recombinase. In some embodiments, the inducible recombinase is unable to translocate to the nucleus in the absence of a second triggering agent. In some embodiments, the third recombination site and the fourth recombination site are oriented in the same direction. In some embodiments, wherein the inducible promoter is not operably linked to the sequence encoding the one or more AAV helper proteins.

In some embodiments, the second polynucleotide comprises a second constitutive promoter operably linked to a sequence encoding an activator, wherein the cell constitutively expresses the activator and the activator is unable to activate an inducible promoter in absence of a first triggering agent. In some embodiments, the second polynucleotide further comprises from 5′ to 3′: (i) the inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; and (ii) a sequence encoding one or more AAV helper proteins, wherein the inducible promoter is not operably linked to the sequence encoding the one or more AAV helper proteins. In some embodiments, the second polynucleotide comprises a third constitutive promoter operably linked to a sequence encoding a second selectable marker, wherein the cell constitutively expresses the second selectable marker.

In some embodiments, in the presence of the first triggering agent, the activator activates the second inducible promoter resulting in expression of the inducible recombinase, and the inducible recombinase is expressed. In some embodiments, in the presence of a second triggering agent, the inducible recombinase translocates to a nucleus of the cell and causes recombination between the third recombination site and the fourth recombination site resulting in excision of the self-excising element, thereby operably linking the second inducible promoter to the sequence encoding the one or more adenoviral helper proteins and allowing expression of the one or more adenoviral helper proteins. FIG. 1B illustrates a schematic of the second polynucleotide after addition of the first triggering agent and the second triggering agent, which allows expression of the helper proteins.

In some embodiments, the one or more adenoviral helper proteins comprise one or more of adenovirus E1A protein, E1B protein, E2A protein, and E4 protein. In certain embodiments, the one or more adenoviral helper proteins comprises E2A protein and E4 protein.

In some embodiments, the second polynucleotide comprising the sequence encoding for one or more AAV helper proteins comprises a bicistronic open reading frame encoding two AAV helper proteins. In certain embodiments, the SEQ ID NO: 29 comprises the second polynucleotide.

In some embodiments, the sequence coding for one or more AAV helper proteins comprises a bicistronic open reading frame encoding two AAV helper proteins. In some embodiments, the two AAV helper proteins are E2A and E4 or E1A and E1B. In some embodiments, wherein the bicistronic open reading frame comprises an internal ribosome entry site (IRES) or a peptide 2A (P2A) sequence.

In some embodiments, the second inducible promoter operably linked to the self-excising element in the second polynucleotide is a tetracycline-inducible promoter, an ecdysone-inducible promoter, or a cumate-inducible promoter.

In some embodiments, the first inducible promoter and the second inducible promoter are the same. In some embodiments, the first inducible promoter and the second inducible promoter are a tetracycline-inducible promoter. In certain embodiments, the tetracycline-inducible promoter comprises a tetracycline-responsive promoter element (TRE). In certain embodiments, the TRE comprises Tet operator (tetO) sequence concatemers fused to a minimal promoter. In certain embodiments, the minimal promoter is a human cytomegalovirus promoter.

In some embodiments, the first constitutive promoter is EF1alpha promoter or human cytomegalovirus promoter.

In some embodiments, the activator is reverse tetracycline-controlled transactivator (rTA) comprising a Tet Repressor binding protein (TetR) fused to a VP16 transactivation domain.

In some embodiments, a triggering agent for inducing the tetracycline-inducible promoter is tetracycline. In other embodiments, a triggering agent for inducing the tetracycline-inducible promoter is doxycycline.

In some embodiments, the inducible recombinase is fused to an estrogen response element (ER) and translocates to the nucleus in the presence of a triggering agent. In some embodiments, the triggering agent for translocating the inducible recombinase is a hormone. In some embodiments, the second triggering agent is tamoxifen.

In some embodiments, the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase. In some embodiments, upon expression of the inducible recombinase in the presence of the second triggering agent, the inducible recombinase translocates to the nucleus, recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins.

In some embodiments, presence of the triggering agent activates the activator for activation of the second inducible promoter to express the Rep proteins of the first polynucleotide, the Cap proteins of the first polynucleotide, the inducible recombinase, and the one or more adenoviral helper proteins.

In some embodiments, upon expression of the inducible recombinase, recombination between the first recombination site and the second recombination site in the first polynucleotide results in excision of the excisable element, and the first part of the AAV Rep proteins coding sequence and the second part of the AAV Rep proteins coding sequence are joined to form a complete AAV Rep proteins coding sequence, wherein the one or more promoters are operably linked to the complete AAV Rep coding sequence to allow expression of an AAV Rep proteins of the first polynucleotide and if present the Cap Proteins of the first polynucleotide; and recombination between the third recombination site and the fourth recombination site in the second polynucleotide results in excision of the self-excising element comprising the sequence encoding the inducible recombinase, wherein the inducible promoter becomes operably linked to the sequence encoding the one or more adenoviral helper proteins to allow expression of the one or more adenoviral helper proteins.

In some embodiments, the second polynucleotide further comprises a second selectable marker operably linked to a third constitutive promoter. In some embodiments, the third constitutive promoter is EF1alpha promoter or human cytomegalovirus promoter. In some embodiments, the EF1alpha promoter is a mutated EF1alpha promoter, such as an EF1alpha promoter comprising a mutation in a TATA box.

In some embodiments, the second polynucleotide is not flanked by inverted terminal repeat sequences.

### VA-RNA

In some embodiments, the second polynucleotide construct further comprises an insert comprising a sequence encoding VA-RNA; or optionally, wherein a fourth construct comprises an insert comprising a sequence encoding VA-RNA. In some embodiments, the VA-RNA is wild-type VA-RNA or VA-RNA comprising one or more mutations in the VA-RNA internal promoter. In some embodiments, the insert comprises: a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and a VA-RNA coding sequence, wherein excision of the second excisable element by the inducible recombinase generates a functional complete second constitutive promoter operably linked to the VA-RNA coding sequence to allow expression of the VA-RNA. In some embodiments, the first part of the second constitutive promoter comprises a distal sequence element (DSE) of an RNA polymerase III promoter, and the second part of the second constitutive promoter comprises a proximal sequence element (PSE) of an RNA polymerase III promoter; the first part of the second constitutive promoter comprises a distal sequence element (DSE) of a U6 promoter, and the second part of the second constitutive promoter comprises a proximal sequence element (PSE) of a U6 promoter; or the first part of the second constitutive promoter comprises a distal sequence element (DSE) of a U7 promoter, and the second part of the second constitutive promoter comprises a proximal sequence element (PSE) of a U7 promoter. In some embodiments, the second polynucleotide construct comprises at least 5%, 10%, 20%, 30%, 40%, 50%, 60%, 70%, 75%, 80%, 85%, 90%, 95%, or 100% sequence identity to SEQ ID NO: 5; SEQ ID NO: 6; SEQ ID NO: 7; SEQ ID NO: 8; SEQ ID NO: 9-SEQ ID NO: 13; SEQ ID NO: 29; SEQ ID NO: 100; or SEQ ID NO: 106.

In some embodiments, the VA-RNA is operably linked to a constitutive promoter or an inducible promoter. In some embodiments, the constitutive promoter is EF1alpha promoter or human cytomegalovirus promoter. In some embodiments, the inducible promoter is a tetracycline-inducible promoter, an ecdysone-inducible promoter, or a cumate-inducible promoter. In some embodiments, a payload construct comprises a polynucleotide construct coding for a VA-RNA, wherein a sequence coding for the VA-RNA comprises at least two mutations in an internal promoter. In some embodiments, a separate polynucleotide construct codes for a VA-RNA, wherein a sequence coding for the VA-RNA comprises at least two mutations in an internal promoter. In some embodiments, the sequence coding for the VA-RNA comprises a sequence coding for a transcriptionally dead VA-RNA. In some embodiments, the sequence coding for the VA-RNA comprises a deletion of from about 5-10 nucleotides in the promoter region. In some embodiments, the sequence coding for the VA-RNA comprises at least one mutation. In some embodiments, the at least one mutation is in the A Box promoter region. In some embodiments, the at least one mutation is in the B Box promoter region. In some embodiments, the at least one mutation is G16A and G60A. In some embodiments, the expression of the VA-RNA is under the control of an RNA polymerase III promoter. In some embodiments, the expression of the VA-RNA is under the control of an interrupted RNA polymerase III promoter. In some embodiments, the expression of the VA-RNA is under the control of a U6 or U7 promoter. In some embodiments, the expression of the VA-RNA is under the control of an interrupted U6 or U7 promoter. In some embodiments, the polynucleotide construct comprises upstream of the VA-RNA gene sequence, from 5′ to 3′: a) a first part of a U6 or U7 promoter sequence; b) a first recombination site; c) a stuffer sequence; d) a second recombination site; e) a second part of a U6 or U7 promoter sequence. In some embodiments, the stuffer sequence is excisable by a recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

In some embodiments, an inducible helper construct comprises a polynucleotide construct coding for a VA-RNA or the VA-RNA construct is in a vector. In some embodiments, an inducible helper construct comprises a polynucleotide construct coding for a VA-RNA or the VA-RNA construct is in a plasmid. In some embodiments, an inducible helper construct comprises a polynucleotide construct coding for a VA-RNA or the VA-RNA construct is in a bacterial artificial chromosome or yeast artificial chromosome. In some embodiments, an inducible helper construct comprises a polynucleotide construct coding for a VA-RNA or the VA-RNA construct is a synthetic nucleic acid construct. In some embodiments, an inducible helper construct comprises a sequence having at least 70%, 80%, 90%, 95%, 99%, or 100% sequence identity to any one of SEQ ID NO: 88-SEQ ID NO: 98. In some embodiments, an inducible helper construct has at least 70%, 80%, 90%, 95%, 99%, or 100% sequence identity to any one of SEQ ID NO: 88-SEQ ID NO: 98. In some embodiments, a VA-RNA construct comprises a sequence having at least 70%, 80%, 90%, 95%, 99%, or 100% sequence identity to any one of SEQ ID NO: 88-SEQ ID NO: 98. In some embodiments, a VA-RNA construct has a sequence having at least 70%, 80%, 90%, 95%, 99%, or 100% sequence identity to any one of SEQ ID NO: 88-SEQ ID NO: 98.

In some embodiments, the stuffer sequence comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the stuffer sequence further encodes a helper enzyme involved in production of tyrosine from phenylalanine. In some embodiments, the helper enzyme facilitates PAH-mediated production of tyrosine from phenylalanine. In some embodiments, the helper enzyme catalyzes production of a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 47. In some embodiments, the GT CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

### Third Polynucleotide Encoding Payload

Returning to FIG. 1A, some embodiments include a third polynucleotide encoding a payload. This third polynucleotide encoding a payload may be referred to as the polynucleotide encoding the payload, the third polynucleotide, Construct 3, payload construct, or therapeutic construct, interchangeably. In some embodiments, the third polynucleotide comprises a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR). The payload may be a gene encoding a polypeptide, such as, an antibody, a hormone, a site-specific endonuclease, a reporter gene, a component of a CRISPR/Cas system, an adenosine deaminase acting on RNA (ADAR) enzyme, a transcriptional activator, a transcriptional repressor, a ribozyme, a DNAzyme, or any combination thereof.

In certain embodiments, the third polynucleotide comprises a reporter gene, a therapeutic gene, and/or a transgene encoding a protein of interest. In certain embodiments, the payload of the third polynucleotide encoding the payload is progranulin.

In some embodiments, the payload within the third polynucleotide comprises a sequence encoding a reporter gene, a therapeutic gene, or a transgene encoding a protein of interest. In some embodiments, the payload within the third polynucleotide comprises a sequence encoding progranulin. In some embodiments, the sequence encoding progranulin has at least 70%, at least 75%, at least 80%, at least 85%, at least 90%, at least 95%, at least 97%, at least 98%, at least 99%, or 100% sequence identity to SEQ ID NO: 30. In certain embodiments, the sequence encoding progranulin has sequence of SEQ ID NO: 30.

In some embodiments, the payload within the third polynucleotide comprises a sequence encoding a suppressor tRNA, a guide RNA, or a homology region for homology-directed repair.

In some embodiments, the third polynucleotide comprises the sequence encoding the payload flanked by a 5′ AAV inverted terminal repeat (5′ ITR) and a 3′ AAV inverted terminal repeat (3′ ITR).

In some embodiments, the sequence encoding the payload is flanked by a 5′ AAV inverted terminal repeat (5′ ITR) and a 3′ AAV inverted terminal repeat (3′ ITR) has at least 70%, at least 75%, at least 80%, at least 85%, at least 90%, at least 95%, at least 97%, at least 98%, at least 99%, or 100% sequence identity to SEQ ID NO: 31. In certain embodiments, the sequence encoding the payload is flanked by a 5′ AAV inverted terminal repeat (5′ ITR) and a 3′ AAV inverted terminal repeat (3′ ITR) has sequence of SEQ ID NO: 31.

In some embodiments, the third polynucleotide has at least 70%, at least 75%, at least 80%, at least 85%, at least 90%, at least 95%, at least 97%, at least 98%, at least 99%, or 100% sequence identity to SEQ ID NO: 32. In certain embodiments, the sequence encoding the payload has sequence of SEQ ID NO: 32.

In some embodiments, the third polynucleotide has at least 70%, at least 75%, at least 80%, at least 85%, at least 90%, at least 95%, at least 97%, at least 98%, at least 99%, or 100% sequence identity to SEQ ID NO: 33. In certain embodiments, the third polynucleotide comprises the sequence of SEQ ID NO: 33. In some embodiments, the payload construct has at least 70%, 80%, 90%, 95%, 99%, or 100% sequence identity to SEQ ID NO: 34.

In some embodiments, the third polynucleotide comprises the coding sequence for an expressible payload and a third mammalian cell selection element. In the exemplary embodiments, the expressible payload is under the control of a constitutive promoter.

In some embodiments, the expressible payload encodes a guide RNA. In certain embodiments, the guide RNA directs RNA editing. In some embodiments, the guide RNA directs Cas-mediated DNA editing. In some embodiments, the guide RNA directs ADAR-mediated RNA editing. In some embodiments, the third polynucleotide comprises a sequence encoding for any of the expressible payloads disclosed herein. For example, said sequence can encode for any therapeutic. For example, the therapeutic may be a transgene, a guide RNA, an antisense RNA, an oligonucleotide, an mRNA, a miRNA, a shRNA, a tRNA suppressor, a CRISPR-Cas protein, any gene editing enzyme, or any combination thereof. In some embodiments, the transgene encodes for progranulin. In some embodiments, the tRNA suppressor is capable of suppressing an opal stop codon. In some embodiments, the tRNA suppressor is capable of suppressing an ochre stop codon. In some embodiments, the tRNA suppressor is capable of suppressing an amber stop codon. In some embodiments, the third integrated synthetic construct comprises sequences encoding for more than one of the expressible payloads disclosed herein. For example, the third integrated synthetic construct comprises 2 gRNA, 3 gRNA, 4 gRNA, 5 gRNA, 6 gRNA, 7 gRNA, 8 gRNA, 9 gRNA, or 10 gRNA. These gRNAs can all be the same, all be different, or any combination of the same and different. For example, the third integrated synthetic construct comprises 2 suppressor tRNAs, 3 suppressor tRNAs, 4 suppressor tRNAs, 5 suppressor tRNAs, 6 suppressor tRNAs, 7 suppressor tRNAs, 8 suppressor tRNAs, 9 suppressor tRNAs, or 10 suppressor tRNAs. These suppressor tRNAs can all be the same, all be different, or any combination of the same and different.

In some embodiments, the expressible payload encodes a protein. In certain embodiments, the expressible payload is an enzyme, useful for replacement gene therapy. In some embodiments, the protein is a therapeutic antibody. In some embodiments, the protein is a vaccine immunogen. In particular embodiments, the vaccine immunogen is a viral protein.

In some embodiments, the expressible payload is a homology construct for homologous recombination.

In various embodiments, the third mammalian cell selection element is an auxotrophic selection element.

In some embodiments, the third polynucleotide comprises at least 5%, 10%, 20%, 30%, 40%, 50%, 60%, 70%, 75%, 80%, 85%, 90%, 95%, or 100% sequence identity to SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 30; SEQ ID NO: 31; SEQ ID NO: 33; SEQ ID NO: 34; SEQ ID NO: 35; SEQ ID NO: 36; SEQ ID NO: 38; SEQ ID NO: 39; SEQ ID NO: 108; or SEQ ID NO: 109; In some embodiments, the sequence encoding progranulin is replaced with a sequence encoding dystrophin. In some embodiments, the sequence encoding dystrophin encodes for a short, functional dystrophin.

In some embodiments, the third polynucleotide comprises a sequence of a payload flanked by ITR sequences. In some embodiments, expression of the sequence of the payload is driven by a constitutive promoter or an inducible promoter. In some embodiments, the promoter and sequence of the payload are flanked by ITR sequences. In some embodiments, the payload construct flanked by ITRs comprises a sequence having at least 70%, 80%, 90%, 95%, 99%, or 100% sequence identity to SEQ ID NO: 31.

In some embodiments, the third polynucleotide comprises a polynucleotide sequence coding for a gene. In some embodiments, the gene codes for a selectable marker or detectable marker. In some embodiments, the gene codes for a therapeutic polypeptide or transgene. In some embodiments, the therapeutic polypeptide or transgene is progranulin. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the tRNA suppressor is capable of suppressing an opal stop codon. In some embodiments, the tRNA suppressor is capable of suppressing an ochre stop codon. In some embodiments, the tRNA suppressor is capable of suppressing an amber stop codon. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the guide RNA, when bound to a target RNA, recruits an ADAR protein for editing of the target RNA. In some embodiments, the Cas protein is catalytically inactive Cas protein. In some embodiments, the polynucleotide encoding the payload is stably integrated into the genome of the cell. In some embodiments, a plurality of polynucleotides encoding the payload are stably integrated into the genome of the cell. In some embodiments, the plurality of polynucleotides encoding the payload are separately stably integrated into the genome of the cell.

A payload may include any one or combination of the following: a transgene, a tRNA suppressor, a guide RNA, or any other target binding/modifying oligonucleotide or derivative thereof, or payloads may include immunogens for vaccines, and elements for any gene editing machinery (DNA or RNA editing). Payloads may also include those that deliver a transgene encoding antibody chains or fragments that are amenable to viral vector-mediated expression (also referred to as “vectored or vectorized antibody” for gene delivery). See, e.g., Curr Opinion HIV AIDS. 2015 May; 10(3): 190-197, describing vectored antibody gene delivery for the prevention or treatment of HIV infection. See also, U.S. Pat. No. 10,780,182, which describes AAV delivery of trastuzumab (Herceptin) for treatment of HER2+ brain metastases. A payload disclosed herein may not be a therapeutic payload (e.g., a coding for a detectable marker such as GFP). In particular, in some instances the polynucleotide payload refers to a polynucleotide that may be a homology element for homology-directed repair, or polynucleotide transcribed into a guide RNA to be delivered for a variety of purposes. In some embodiments, the transgene refers to a nucleic acid sequence coding for expression of guide RNA for ADAR editing or ADAT editing. In some embodiments, the transgene refers to a transgene packaged for gene therapy. In some embodiments, the transgene refers to synthetic constructs packaged for vaccines. In certain aspects, a polynucleotide payload may be described as encoding an RNA, which is meant to refer to the RNA transcribed from the polynucleotide.

In certain examples, the third polynucleotide comprises two expressible sequences, wherein a first expressible sequence encodes for a first gRNA and a second expressible sequence encodes for a second gRNA. In some embodiments, the first gRNA and the second gRNA are different. In some embodiments, the first gRNA and the second gRNA are the same. In certain examples, the third polynucleotide comprises two or more expressible sequences. In some embodiments, the two or more expressible sequences encode for two or more gRNA. In some embodiments, the two or more gRNA are all different gRNA, all the same gRNA, or a combination of the same and different gRNA.

In some cases, the third polynucleotide comprises an expressible sequence encoding both a heterologous RNA and a heterologous polypeptide. In other cases, the expressible sequence encodes two or more heterologous payloads. Where the expressible sequence encodes two heterologous payloads, in some cases, the nucleotide sequences encoding the two heterologous payloads are operably linked to the same promoter. Where the expressible sequence encodes two heterologous payloads, in some cases, the nucleotide sequences encoding the two heterologous payloads are operably linked to two different promoters. In some cases, sequence encoding the payload comprises an expressible sequence encoding three heterologous payloads. Where the expressible sequence encodes three heterologous payloads, in some cases, the nucleotide sequences encoding the three heterologous payloads are operably linked to the same promoter. Where the expressible sequence encodes three heterologous payloads, in some cases, the nucleotide sequences encoding the three heterologous payloads are operably linked to two or three different promoters. In some cases, the third polynucleotide construct of the present disclosure comprises two or more expressible sequences, each comprising a nucleotide sequence encoding a heterologous payload.

In some embodiments, the expressible sequence encodes a polypeptide of interest. The polypeptide of interest may be any type of protein/peptide including, without limitation, an enzyme, an extracellular matrix protein, a receptor, transporter, ion channel, or other membrane protein, a hormone, a neuropeptide, an antibody, or a cytoskeletal protein; or a fragment thereof, or a biologically active domain of interest. In some cases, the payload is a therapeutic polypeptide, e.g., a polypeptide that provides clinical benefit.

Where the payload is an interfering RNA (RNAi), suitable RNAi include RNAi that decrease the level of an apoptotic or angiogenic factor in a cell. For example, an RNAi may be an shRNA or siRNA that reduces the level of a payload that induces or promotes apoptosis in a cell. A payload may be a gene whose gene product induces or promotes apoptosis are referred to herein as “pro-apoptotic genes” and the products of those genes (mRNA; protein) are referred to as “pro-apoptotic gene products.” Pro-apoptotic gene products include, e.g., Bax, Bid, Bak, and Bad gene products. See, e.g., U.S. Pat. No. 7,846,730. In another example, the RNAi specifically reduces the level of an RNA and/or a polypeptide product of a defective allele.

In some embodiments, the payload is an aptamer. In some cases, the aptamer is a therapeutic aptamer. For example, the aptamer may function as an antagonist by blocking interactions at a disease-associated target (e.g., receptor-ligand interactions). Alternatively, an aptamer may serve as an agonist for activating the function of a target receptor. Exemplary aptamers of interest include aptamers against growth factor receptors and growth factors such as aptamers that bind to epidermal growth factor receptor (see, e.g., Wang et al. (2014) Biochem. Biophys. Res. Commun. 453(4):681-5), transforming growth factor-beta type III receptor (see, e.g., Ohuchi et al. (2006) Biochimie 88(7):897-904.), vascular endothelial growth factor (VEGF) (see, e.g., Ng et al. (2006) Nat. Rev. Drug Discovery 5:123; and Lee et al. (2005) Proc. Natl. Acad. Sci. USA 102:18902) or platelet-derived growth factor (PDGF), e.g., E10030 (see, e.g., Ni and Hui (2009) Ophthalmologica 223:401; and Akiyama et al. (2006) J. Cell Physiol. 207:407).

In some embodiments, the expressible sequence encodes a sequence-specific endonuclease for use in genome editing. The sequence specific endonuclease may be used to create a double-stranded break at a specific site in the genome. The double stranded breaks may then be repaired by non-homologous end joining (NHEJ), microhomology-mediated end joining (MMEJ), or homology-directed repair (HDR) pathways. Desired genome edits may be introduced into the genome using donor DNA to repair double-strand breaks by homologous recombination. Various sequence-specific endonucleases may be used in genome editing for creation of double-strand breaks in DNA, including, without limitation, engineered zinc-finger nucleases (ZFNs), transcription activator-like effector nucleases (TALENs), meganucleases, and clustered regularly interspaced short palindromic repeats (CRISPR) Cas9. See, e.g., Targeted Genome Editing Using Site-Specific Nucleases: ZFNs, TALENs, and the CRISPR/Cas9 System (T. Yamamoto ed., Springer, 2015); Genome Editing: The Next Step in Gene Therapy (Advances in Experimental Medicine and Biology, T. Cathomen, M. Hirsch, and M. Porteus eds., Springer, 2016); Aachen Press Genome Editing (CreateSpace Independent Publishing Platform, 2015); herein incorporated by reference. Precise control over the timing of production of the genome editing enzyme may be achieved by inducibly producing recombinant adenovirus associated virus (rAAV) virions with the vector system to allow turning on and off of expression as desired.

In some cases, a payload of interest is a site-specific endonuclease that provides for site-specific knock-down of gene function, e.g., where the endonuclease knocks out an allele associated with a disease. For example, in a case where a dominant allele encodes a defective copy of a gene, and the wild-type gene provides for normal function, a site-specific endonuclease may be targeted to the defective allele and knock out the defective allele. In some cases, a site-specific endonuclease is an RNA-guided endonuclease.

A site-specific nuclease may also be used to stimulate homologous recombination with a donor DNA that encodes a functional copy of the protein encoded by the defective allele. Thus, e.g., a subject rAAV virion may be used to deliver a site-specific endonuclease that knocks out a defective allele and also be used to deliver a functional copy of the defective allele, resulting in repair of the defective allele, thereby providing for production of a functional gene product.

In some cases, the payload is an RNA-guided endonuclease. In some cases, the payload is an RNA comprising a nucleotide sequence encoding an RNA-guided endonuclease. In some cases, the payload is a guide RNA, e.g., a single-guide RNA. In some cases, the payloads are: 1) a guide RNA; and 2) an RNA-guided endonuclease. The guide RNA may comprise: a) a protein-binding region that binds to the RNA-guided endonuclease; and b) a region that binds to a target nucleic acid. An RNA-guided endonuclease is also referred to herein as a “genome editing nuclease.”

Examples of RNA-guided endonucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a suitable RNA-guided endonuclease is a class 2 CRISPR/Cas endonuclease. In some cases, a suitable RNA-guided endonuclease is a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a suitable RNA-guided endonuclease is a class 2 type VI CRISPR/Cas endonuclease (e.g., a C2c2 protein; also referred to as a “Cas13a” protein). Also suitable for use is a CasX protein. Also suitable for use is a CasY protein.

In some cases, the genome-editing endonuclease is a Type II CRISPR/Cas endonuclease. In some cases, the genome-editing endonuclease is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, the Cas9 polypeptide used in a composition or method of the present disclosure is a Staphylococcus aureus Cas9 (saCas9) polypeptide. In some cases, a suitable Cas9 polypeptide is a high-fidelity (HF) Cas9 polypeptide. Kleinstiver et al. (2016) Nature 529:490. In some cases, a suitable Cas9 polypeptide exhibits altered PAM specificity. See, e.g., Kleinstiver et al. (2015) Nature 523:481. In some cases, the genome-editing endonuclease is a type V CRISPR/Cas endonuclease. In some cases a type V CRISPR/Cas endonuclease is a Cpf1 protein. In some cases, the genome-editing endonuclease is a CasX or a CasY polypeptide. CasX and CasY polypeptides are described in Burstein et al. (2017) Nature 542:237.

In some cases, a genome editing nuclease is a fusion protein that is fused to a heterologous polypeptide (also referred to as a “fusion partner”). In some cases, a genome editing nuclease is fused to an amino acid sequence (a fusion partner) that provides for subcellular localization, i.e., the fusion partner is a subcellular localization sequence (e.g., one or more nuclear localization signals (NLSs) for targeting to the nucleus, two or more NLSs, three or more NLSs, etc.).

Also suitable for use is an RNA-guided endonuclease with reduced enzymatic activity. Such an RNA-guided endonuclease is referred to as a “dead” RNA-guided endonuclease; for example, a Cas9 polypeptide that comprises certain amino acid substitutions such that it exhibits substantially no endonuclease activity, but such that it still binds to a target nucleic acid when complexed with a guide RNA, is referred to as a “dead” Cas9 or “dCas9.” In some cases, a “dead” Cas9 protein has a reduced ability to cleave both the complementary and the non-complementary strands of a double stranded target nucleic acid. For example, a “nuclease defective” Cas9 lacks a functioning RuvC domain (i.e., does not cleave the non-complementary strand of a double stranded target DNA) and lacks a functioning HNH domain (i.e., does not cleave the complementary strand of a double stranded target DNA). Such a Cas9 protein has a reduced ability to cleave a target nucleic acid (e.g., a single stranded or double stranded target nucleic acid) but retains the ability to bind a target nucleic acid. A Cas9 protein that may not cleave target nucleic acid (e.g., due to one or more mutations, e.g., in the catalytic domains of the RuvC and HNH domains) is referred to as a “nuclease defective Cas9”, “dead Cas9” or simply “dCas9.” Other residues may be mutated to achieve the above effects (i.e. inactivate one or the other nuclease portions).

In some cases, the genome-editing endonuclease is an RNA-guided endonuclease (and its corresponding guide RNA) known as Cas9-synergistic activation mediator (Cas9-SAM). The RNA-guided endonuclease (e.g., Cas9) of the Cas9-SAM system is a “dead” Cas9 fused to a transcriptional activation domain (wherein suitable transcriptional activation domains include, e.g., VP64, p65, MyoD1, HSF1, RTA, and SET7/9) or a transcriptional repressor domain (where suitable transcriptional repressor domains include, e.g., a KRAB domain, a NuE domain, an NcoR domain, a SID domain, and a SID4X domain). The guide RNA of the Cas9-SAM system comprises a loop that binds an adapter protein fused to a transcriptional activator domain (e.g., VP64, p65, MyoD1, HSF1, RTA, or SET7/9) or a transcriptional repressor domain (e.g., a KRAB domain, a NuE domain, an NcoR domain, a SID domain, or a SID4X domain). For example, in some cases, the guide RNA is a single-guide RNA comprising an MS2 RNA aptamer inserted into one or two loops of the sgRNA; the dCas9 is a fusion polypeptide comprising dCas9 fused to VP64; and the adaptor/functional protein is a fusion polypeptide comprising: i) MS2; ii) p65; and iii) HSF1. See, e.g., U.S. Patent Publication No. 2016/0355797.

Also suitable for use is a chimeric polypeptide comprising: a) a dead RNA-guided endonuclease; and b) a heterologous fusion polypeptide. Examples of suitable heterologous fusion polypeptides include a polypeptide having, e.g., methylase activity, demethylase activity, transcription activation activity, transcription repression activity, transcription release factor activity, histone modification activity, RNA cleavage activity, DNA cleavage activity, DNA integration activity, or nucleic acid binding activity.

A nucleic acid that binds to a class 2 CRISPR/Cas endonuclease (e.g., a Cas9 protein; a type V or type VI CRISPR/Cas protein; a Cpf1 protein; etc.) and targets the complex to a specific location within a target nucleic acid is referred to herein as a “guide RNA” or “CRISPR/Cas guide nucleic acid” or “CRISPR/Cas guide RNA.” A guide RNA provides target specificity to the complex (the RNP complex) by including a targeting segment, which includes a guide sequence (also referred to herein as a targeting sequence), which is a nucleotide sequence that is complementary to a sequence of a target nucleic acid.

In some cases, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some cases, the guide RNA is one molecule (e.g., for some class 2 CRISPR/Cas proteins, the corresponding guide RNA is a single molecule; and in some cases, an activator and targeter are covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, the guide RNA is at least partially complementary to a target RNA sequence and is capable of recruiting an ADAR enzyme for RNA editing of the target RNA sequence.

Where the payload is an RNA-guided endonuclease or is both an RNA-guided endonuclease and a guide RNA, the payload may modify a target nucleic acid. In some cases, e.g., where a target nucleic acid comprises a deleterious mutation in a defective allele (e.g., a deleterious mutation in a neural cell target nucleic acid), the RNA-guided endonuclease/guide RNA complex, together with a donor nucleic acid comprising a nucleotide sequence that corrects the deleterious mutation (e.g., a donor nucleic acid comprising a nucleotide sequence that encodes a functional copy of the protein encoded by the defective allele), may be used to correct the deleterious mutation, e.g., via homology-directed repair (HDR).

In some cases, the payloads are an RNA-guided endonuclease and 2 separate sgRNAs, where the 2 separate sgRNAs provide for deletion of a target nucleic acid via non-homologous end joining (NHEJ).

In some cases, the payloads are: i) an RNA-guided endonuclease; and ii) one guide RNA. In some cases, the guide RNA is a single-molecule (or “single guide”) guide RNA (an “sgRNA”). In some cases, the guide RNA is a dual-molecule (or “dual-guide”) guide RNA (“dgRNA”).

In some cases, the payloads are: i) an RNA-guided endonuclease; and ii) 2 separate sgRNAs, where the 2 separate sgRNAs provide for deletion of a target nucleic acid via non-homologous end joining (NHEJ). In some cases, the guide RNAs are sgRNAs. In some cases, the guide RNAs are dgRNAs.

In some cases, the payloads are: i) a Cpf1 polypeptide; and ii) a guide RNA precursor; in these cases, the precursor may be cleaved by the Cpf1 polypeptide to generate 2 or more guide RNAs.

The payloads as described herein may be flanked by ITRs.

In some embodiments, the third polynucleotide further comprises a sequence coding for a selectable marker or detectable marker outside of the ITR sequences. In some embodiments, expression of the selectable marker or detectable marker outside of the ITR sequences is driven by a promoter. The promoter can be a constitutive promoter or an inducible promoter. In some embodiments, the constitutive promoter is EF1alpha promoter or human cytomegalovirus promoter. In some embodiments, the inducible promoter is a tetracycline-inducible promoter, an ecdysone-inducible promoter, or a cumate-inducible promoter. In some embodiments, the selectable marker is a mammalian cell selection element (e.g., a third mammalian cell selection element).

In some embodiments, the selectable marker is outside of the ITR sequences on the payload construct. In some embodiments, the payload construct further comprises a spacer between the 5′ ITR and the promoter/selectable marker or promoter/detectable marker outside of the ITR sequences. In some embodiments, the payload construct further comprises a spacer between the 3′ ITR and the promoter/selectable marker or promoter/detectable marker outside of the ITR sequences. In some embodiments, the spacer ranges in length from 500 base pairs to 5000 base pairs, including any length within this range such as 500, 525, 550, 575, 600, 625, 650, 675, 700, 725, 750, 775, 800, 825, 850, 875, 900, 925, 950, 975, 1000, 1250, 1500, 1750, 2000, 2225, 2500, 2750, 3000, 3250, 3500, 3750, 4000, 4250, 4500, 4750, or 5000 base pairs. In some embodiments, the spacer length is a sufficient length for decreasing reverse packaging of the selectable marker or detectable marker that is outside the ITR sequences.

In some embodiments, the third polynucleotide comprising the coding sequence for a payload and a selectable marker or detectable marker is further engineered to remove locations having the potential for Rep-mediated nicking. For example, a location having the potential for Rep-mediated nicking is a location having the sequence CAGTGAGCGAGCGAGCGCGCAG (SEQ ID NO: 85); a sequence comprising GAGC (SEQ ID NO: 86) repeats; or the sequence GATGGAGTTGGCCACTCCCTC (SEQ ID NO: 87). These sequences can be engineered to prevent binding of Rep proteins for Rep-mediated nicking. In some embodiments, the location having the potential for Rep-mediated nicking that is engineered to prevent binding of Rep proteins for Rep-mediated nicking is in a region within 100 nucleotides of an ITR sequence. In some embodiments, the location having the potential for Rep-mediated nicking that is engineered to prevent binding of Rep proteins for Rep-mediated nicking is in a region within 200 nucleotides of an ITR sequence. In some embodiments, the location having the potential for Rep-mediated nicking that is engineered to prevent binding of Rep proteins for Rep-mediated nicking is in a region within 300 nucleotides of an ITR sequence. In some embodiments, the location having the potential for Rep-mediated nicking that is engineered to prevent binding of Rep proteins for Rep-mediated nicking is in a region within 400 nucleotides of an ITR sequence. In some embodiments, the location having the potential for Rep-mediated nicking that is engineered to prevent binding of Rep proteins for Rep-mediated nicking is in a region within 500 nucleotides of an ITR sequence. In some embodiments, the location having the potential for Rep-mediated nicking that is engineered to prevent binding of Rep proteins for Rep-mediated nicking is in a region within 600, 625, 650, 675, 700, 725, 750, 775, 800, 825, 850, 875, 900, 925, 950, 975, 1000, 1250, 1500, 1750, 2000, 2225, 2500, 2750, 3000, 3250, 3500, 3750, 4000, 4250, 4500, 4750, or 5000 nucleotides of an ITR sequence.

### Selectable Markers for Polynucleotides

In some embodiments, a) the first polynucleotide further comprises a sequence encoding a first selectable marker operably linked to a first promoter.

In some embodiments, b) the second polynucleotide further comprises a sequence encoding a second selectable marker operably linked to a second promoter.

In some embodiments, c) the third polynucleotide further comprises a sequence encoding a third selectable marker operably linked to a third promoter.

In some embodiments, the system of polynucleotides comprises any combinations of the first selectable marker, the second selectable marker, and the third selectable marker.

In some embodiments, any combinations of the first selectable marker, the second selectable marker, and the third selectable marker are different selectable markers; the first selectable marker, the second selectable marker, the third selectable marker, or any combinations thereof are the same selectable marker but as a different split portion of the selectable marker; or any combinations thereof.

In some embodiments, any combinations of the first promoter, the second promoter, and the third promoter, are the same constitutive promoter or different constitutive promoters.

In some embodiments, the first selectable marker, the second selectable marker, the third selectable marker, or any combination thereof is an antibiotic resistance gene. In certain embodiments, the antibiotic resistance gene is a blasticidin resistance gene, a hygromycin resistance gene, or a puromycin resistance gene.

In some embodiments, the first selectable marker, the second selectable marker, the third selectable marker, or any combination thereof is a first split portion of an antibiotic resistance gene. In certain embodiments, the first split portion of the antibiotic resistance gene is a first split portion of the blasticidin resistance gene.

In some embodiments, the first selectable marker, the second selectable marker, the third selectable marker, or any combination thereof is a second split portion of an antibiotic resistance gene. In certain embodiments, the second split portion of the antibiotic resistance gene is a second split portion of the blasticidin resistance gene.

In some embodiments, the first promoter, the second promoter, the third promoter, or any combination thereof, is an EF1alpha promoter or an attenuated version thereof. In some embodiments, the attenuated version comprises a mutation in the TATA box. In certain embodiments, the attenuated EF1alpha promoter has weaker promoter activity than an EF1alpha promoter.

In some embodiments, (i) the first polynucleotide further comprises a constitutive promoter operably linked to a sequence encoding a first portion of a split selectable marker; (ii) the second polynucleotide further comprises a sequence encoding a selectable marker operably linked to a constitutive promoter; and/or (iii) the third polynucleotide further comprises a constitutive promoter operably linked to a sequence encoding a selectable marker or a second part of the split selectable marker. In some embodiments, the constitutive promoter is an EF1 alpha promoter and/or the split selectable marker is a split antibiotic resistance protein. In some embodiments, the constitutive promoter is an EF1 alpha promoter and/or the selectable marker is a first antibiotic resistance protein.

In some embodiments, the constitutive promoter is an CMV promoter and/or the selectable marker is a second antibiotic resistance protein. In some embodiments, the constitutive promoter is an EF1 alpha promoter.

In some embodiments, the third polynucleotide comprising the sequence encoding the payload further comprises a spacer between the 5′ ITR and the sequence encoding the selectable marker or a spacer between the sequence encoding the fourth selectable marker and the 3′ ITR, or a combination thereof. In some embodiments, the spacer ranges in length from 500 base pairs to 5000 base pairs.

### Selectable Marker

In some embodiments, suitable markers include genes which confer resistance to antibiotics or toxins, or sensitivity, or impart color, or change the antigenic characteristics when cells, which have been transfected with the nucleic acid constructs, are grown in an appropriate selective medium. Exemplary selectable marker genes include, without limitation, the neomycin resistance gene (neo encoding aminoglycoside phosphotransferase (APH)) that allows selection in mammalian cells by conferring resistance to G418 (Geneticin), the hygromycin-B resistance gene (hygB encoding hygromycin-B-phosphotransferase (HPH)) that confers resistance to hygromycin-B, the puromycin resistance gene (pac encoding puromycin-N-acetyltransferase) that confers resistance to puromycin, Zeocin resistance gene (Sh bla encodes a protein that binds to Zeocin) that prevents Zeocin from binding DNA and damaging it, and the blasticidin resistance gene (BSD) that confers resistance to blasticidin. In addition, dihydrofolate reductase (DHFR)-based methotrexate (MTX) selection or glutamine synthetase (GS)-based methionine sulfoximine (MSX) selection may be used in mammalian cells. Other suitable markers and selection methods are known to those of skill in the art.

In some embodiments, the selectable marker is an antibiotic resistance protein. In some embodiments, the selectable marker is a split selectable marker that allows for selection of cells retaining two different polynucleotides using a single selective pressure. In some embodiments, the antibiotic resistance protein is split into two portions that can associate to form a functional antibiotic resistance protein. A first portion of the antibiotic resistance protein is encoded by a first polynucleotide and a second portion of the antibiotic resistance protein is encoded by a second polynucleotide. In some embodiments, a split intervening proteins (inteins) system that permits stable retention of two integrated nucleic acid constructs under a single selective pressure is used. Inteins auto catalyze a protein splicing reaction that results in excision of the intein and joining of the flanking amino acids (extein sequences) via a peptide bond. Inteins exist in nature as a single domain within a host protein or, less frequently, in a split form. For split inteins, the two separate polypeptide fragments of the intein must associate in order for protein trans-splicing to occur to excise the intein. Split intein systems are described in: Cheriyan et al, J. Biol. Chem 288: 6202-6211 (2013); Stevens et al, PNAS 114: 8538-8543 (2017); Jillette et al., Nat Comm 10: 4968 (2019); US 2020/0087388 A1; and US 2020/0263197 A1. In some embodiments, a split intein is derived from the Nostoc punctiforme (Npu) DnaE intein, the Synechocystis species, strain PCC6803 (Ssp) DnaE intein, or the consensus DnaE intein (Cfa). In some embodiments, a first portion of the antibiotic resistance protein is the N-terminal portion which is fused to a N-terminal intein at the C-terminus and a second portion of the antibiotic resistance protein is the C-terminal portion which is fused to a C-terminal intein at the N-terminus. When both portions are present, the N-terminal intein associates with the C-terminal intein resulting in excision of the inteins and splicing of the C-terminus of the N-terminal portion of the antibiotic resistance protein to the N-terminus of the C-terminal portion of the antibiotic resistance protein, thereby forming a functional antibiotic resistance proteins.

In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 40. In some embodiments, GS comprises at least 80%, 85%, 90%, 95%, 98%, 99%, or 100% sequence identity to SEQ ID NO: 41. In some embodiments, TYMS comprises at least 80%, 85%, 90%, 95%, 98%, 99%, or 100% sequence identity to SEQ ID NO: 42. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter or vice a versa. In some embodiments, the auxotrophic selection element codes for DHFR Z-Cter and DHFR Z-Nter. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein.

In certain embodiments, a split auxotrophic selection system that permits stable retention of two integrated nucleic acid constructs under a single selective pressure can be used. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine.

In some embodiments, the DHFR Z-Nter comprises a sequence having at least 70%, 80%, 90%, 95%, 99%, or 100% sequence identity to SEQ ID NO: 43. In some embodiments, the DHFR Z-Cter comprises a sequence having at least 70%, 80%, 90%, 95%, 99%, or 100% sequence identity to SEQ ID NO: 44. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 45. In some embodiments, a C-terminal intein comprises at least 80%, 85%, 90%, 95%, 98%, 99%, or 100% sequence identity to SEQ ID NO: 46.

In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 40. In some embodiments, GS comprises at least 80%, 85%, 90%, 95%, 98%, 99%, or 100% sequence identity to SEQ ID NO: 41. In some embodiments, TYMS comprises at least 80%, 85%, 90%, 95%, 98%, 99%, or 100% sequence identity to SEQ ID NO: 42. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments, the selectable marker is DHFR Z-Nter or DHFR Z-Cter.

In some embodiments, the payload construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the payload construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 47. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 40-SEQ ID NO: 43 or SEQ ID NO: 48-SEQ ID NO: 75. In some embodiments, the selectable marker and helper enzyme comprises at least 80%, 85%, 90%, 95%, 98%, 99%, or 100% sequence identity to SEQ ID NO: 76-SEQ ID NO: 84.

### Detectable Marker

In some embodiments, the detectable marker comprises a luminescent marker or a fluorescent marker. In some embodiments, the fluorescent marker is GFP, EGFP, RFP, CFP, BFP, YFP, or mCherry.

### Embodiments of Polynucleotide System

In some embodiments, the system of polynucleotide comprises the first polynucleotide described in section “First Polynucleotide Encoding Rep and/or Cap” above; the second polynucleotide described in section “Second Polynucleotide Encoding Helper Proteins” above; and the third polynucleotide described in sections “Third Polynucleotide Encoding Payload” above.

In some aspects, systems of polynucleotides comprising: (a) the polynucleotide of any one of the embodiments disclosed here, wherein the polynucleotide is a first polynucleotide; and one or more of: b) a second polynucleotide comprising a sequence encoding one or more adenoviral helper proteins; and c) a third polynucleotide comprising a sequence encoding a payload are provided.

In some embodiments, the second polynucleotide comprising the sequence encoding one or more adenoviral helper proteins comprises: an inducible promoter operably linked to a self-excising element, wherein the inducible promoter is a third inducible promoter; the self-excising element comprising a third recombination site and a fourth recombination site flanking a sequence encoding an inducible recombinase, wherein the third recombination site and the fourth recombination site are oriented in the same direction, wherein the third inducible promoter is not operably linked to the sequence encoding the one or more AAV helper proteins; a constitutive promoter operably linked to a sequence encoding an activator, wherein a cell comprising the second polynucleotide constitutively expresses the activator and the activator is unable to activate the first inducible promoter, if present the second inducible promoter, or the third inducible promoter in absence of a first triggering agent; wherein in absence of activation of the first inducible promoter, if present the second inducible promoter, and the third inducible promoter, the cell does not express detectable levels of the Rep proteins or the Cap proteins from the first polynucleotide, the inducible recombinase, and the one or more AAV helper proteins, and wherein the inducible recombinase is activated in the presence of a second triggering agent.

In some embodiments, the one or more helper proteins comprise one or more of adenovirus E1A protein, E1B protein, E2A protein, and E4 protein, and optionally comprises E2A protein and E4 protein.

In some embodiments, the third polynucleotide comprising the sequence encoding the payload further comprises: a selectable marker or a second part or a first part of the split selectable marker operably linked to a constitutive promoter and the sequence encoding the payload is flanked by a 5′ AAV inverted terminal repeat (5′ ITR) and a 3′ AAV inverted terminal repeat (3′ ITR); optionally, wherein the sequence encoding the payload is flanked by a 5′ AAV inverted terminal repeat (5′ ITR) and a 3′ AAV inverted terminal repeat (3′ ITR) has at least 70%, at least 75%, at least 80%, at least 85%, at least 90%, at least 95%, at least 97%, at least 98%, at least 99%, or 100% sequence identity to SEQ ID NO: 31 or SEQ ID NO: 34.

In some embodiments, the third polynucleotide comprising the sequence encoding the payload further comprises further comprises a spacer between the 5′ ITR and the sequence encoding the selectable marker or a spacer between the sequence encoding the selectable marker and the 3′ ITR, or a combination thereof; optionally wherein SEQ ID NO: 31 or SEQ ID NO: 34 comprises the fourth polynucleotide.

In some embodiments, the second polynucleotide comprises a sequence encoding a viral associated RNA (VA-RNA); optionally, wherein the VA-RNA is a mutated VA-RNA.

In some embodiments, the VA-RNA is wild-type VA-RNA or VA-RNA comprising one or more mutations in the VA-RNA internal promoter.

In some embodiments, the sequence encoding the VA-RNA is operably linked to an inactive promoter comprising a first part of a constitutive promoter and a second part of the constitutive promoter separated by a second excisable element comprising a fifth recombination site and a sixth recombination site flanking a stuffer sequence, the fifth and sixth recombination sites are oriented in the same direction, and excision of the second excisable element by the inducible recombinase generates a functional complete constitutive promoter operably linked to the VA-RNA coding sequence to allow expression of the VA-RNA.

In some embodiments, the first part of the constitutive promoter comprises a distal sequence element (DSE) of an RNA polymerase III promoter, and the second part of the constitutive promoter comprises a proximal sequence element (PSE) of an RNA polymerase III promoter, or the first part of the constitutive promoter comprises a distal sequence element (DSE) of a U6 promoter, and the second part of the constitutive promoter comprises a proximal sequence element (PSE) of a U6 promoter, or the first part of the constitutive promoter comprises a distal sequence element (DSE) of a U7 promoter, and the second part of the constitutive promoter comprises a proximal sequence element (PSE) of a U7 promoter.

In some embodiments, the VA-RNA comprises a G16A mutation or a G60A mutation, or a combination thereof.

In some embodiments, (i) the first polynucleotide comprising the sequence encoding the first sequence encoding AAV Rep proteins and the second sequence encoding AAV Cap proteins comprises a constitutive promoter operably linked to a sequence encoding a first portion of a split selectable marker, optionally, wherein the constitutive promoter is an EF1 alpha promoter and/or the split selectable marker is a split antibiotic resistance protein; (ii) the second polynucleotide comprising the sequence encoding one or more adenoviral helper proteins comprises a sequence encoding a selectable marker operably linked to a constitutive promoter, optionally, wherein the constitutive promoter is an CMV promoter and/or the selectable marker is a second antibiotic resistance protein; and/or (iii) the third polynucleotide comprising the sequence encoding the payload comprises a constitutive promoter operably linked to a sequence encoding a selectable marker or a second part of the split selectable marker, optionally, wherein the constitutive promoter is an EF1 alpha promoter.

### Synthetic Nucleic Acid Constructs

In typical embodiments, the nuclear genome of the cell of the stable cell line comprises a plurality of polynucleotides (e.g., first polynucleotide, second polynucleotide, third polynucleotide, etc.). In some embodiments, each of the plurality of polynucleotides is separately integrated into the nuclear genome of the cell. In some embodiments, only a single non-auxotrophic selection is required to maintain all of the plurality of synthetic nucleic acid constructs stably within the nuclear genome of the cells. In some embodiments, antibiotic resistance is required to maintain the plurality of polynucleotides stably within the nuclear genomes of the cells. In some embodiments, both a non-auxotrophic selection and antibiotic resistance is required to maintain the plurality of polynucleotides stably within the nuclear genomes of the cells. In some embodiments, auxotrophic selection and antibiotic resistance is required to maintain the plurality of synthetic constructs stably within the nuclear genomes of the cells. In some embodiments, auxotrophic selection is required to maintain the plurality of polynucleotides stably within the nuclear genomes of the cells.

In particular embodiments, the first polynucleotides comprises conditionally expressible AAV Rep and Cap coding sequences; the second polynucleotides comprises a conditionally expressible Cre coding sequence and conditionally expressible adenoviral helper protein coding sequences; and the third polynucleotides comprises expressible coding sequences for the payload.

In particular embodiments, the first polynucleotides comprises conditionally expressible AAV Rep and Cap coding sequences, where the AAV Cap coding sequence is operably linked to an inducible promoter; the second polynucleotides comprises a conditionally expressible Cre coding sequence and conditionally expressible adenoviral helper protein coding sequences; and the third polynucleotides comprises expressible coding sequences for the payload.

In some aspects, vectors comprising the polynucleotide described in the present disclosure are provided. the first polynucleotide described in section “First Polynucleotide Encoding Rep and/or Cap” above; the second polynucleotide described in section “Second Polynucleotide Encoding Helper Proteins” above; and the third polynucleotide described in sections “Third Polynucleotide Encoding Payload” above.

In some embodiments, a vector system comprises: a) a first vector comprising the sequence of the first polynucleotide described in section “First Polynucleotide Encoding Rep and/or Cap” above; b) a second vector comprising the sequence of the second polynucleotide described in section “Second Polynucleotide Encoding Helper Proteins” above; and c) a third vector comprising the sequence of the third polynucleotide described in sections “Third Polynucleotide Encoding Payload” above.

In some embodiments, the first vector is a first plasmid, the second vector is a second plasmid, and the third vector is a third plasmid.

In some embodiments, the first plasmid has least 70%, at least 75%, at least 80%, at least 85%, at least 90%, at least 95%, at least 97%, at least 98%, at least 99%, or 100% sequence identity to SEQ ID NO: 99 or SEQ ID NO: 22 or 23 In certain embodiments, the first plasmid has sequence of SEQ ID NO: 22, SEQ ID NO: 23, SEQ ID NO: 99, SEQ ID NO: 101, or SEQ ID NO: 102.

In some embodiments, the second plasmid has at least 70%, at least 75%, at least 80%, at least 85%, at least 90%, at least 95%, at least 97%, at least 98%, at least 99%, or 100% sequence identity to SEQ ID NO: 13; SEQ ID NO: 29; SEQ ID NO: 100; or SEQ ID NO: 106. In certain embodiments, the second plasmid has sequence of SEQ ID NO: 13; SEQ ID NO: 29; SEQ ID NO: 100; or SEQ ID NO: 106.

In some embodiments, the third plasmid has at least 70%, at least 75%, at least 80%, at least 85%, at least 90%, at least 95%, at least 97%, at least 98%, at least 99%, or 100% sequence identity to SEQ ID NO: 39 or SEQ ID NO: 39. In certain embodiments, the third plasmid has sequence of SEQ ID NO: 38 or sequence of SEQ ID NO: 39.

In some aspects, vector systems comprising: a) a first vector comprising the sequence of the first polynucleotide described in section “First Polynucleotide Encoding Rep and/or Cap” above and one or more of: b) a second vector comprising the sequence of the second polynucleotide described in section “Second Polynucleotide Encoding Helper Proteins” above; and c) a third vector comprising the sequence of the third polynucleotide described in sections “Third Polynucleotide Encoding Payload” above are provided.

In some embodiments, the first vector is a first plasmid, the second vector is a second plasmid, and the third vector is a third plasmid.

In some embodiments, the first plasmid has at least 70%, at least 75%, at least 80%, at least 85%, at least 90%, at least 95%, at least 97%, at least 98%, at least 99%, or 100% sequence identity to SEQ ID NO: 101 or 102. In certain embodiments, the first plasmid has sequence of SEQ ID NO: 101 or 102.

In some embodiments, the second plasmid has at least 70%, at least 75%, at least 80%, at least 85%, at least 90%, at least 95%, at least 97%, at least 98%, at least 99%, or 100% sequence identity to SEQ ID NO: 13; SEQ ID NO: 29; SEQ ID NO: 100; or SEQ ID NO: 106. In certain embodiments, the second plasmid has sequence of SEQ ID NO: 29 or sequence of SEQ ID NO: 13; SEQ ID NO: 29; SEQ ID NO: 100; or SEQ ID NO: 106.

In some embodiments, the third plasmid has at least 70%, at least 75%, at least 80%, at least 85%, at least 90%, at least 95%, at least 97%, at least 98%, at least 99%, or 100% sequence identity to SEQ ID NO: 38 or SEQ ID NO: 39. In certain embodiments, the third plasmid has sequence of SEQ ID NO: 38 or sequence of SEQ ID NO: 39.

Production of Single-Stranded or Self-Complementary rAAV Virion DNA

In some embodiments, the region of the third polynucleotide construct between the two inverted terminal repeats (3′ ITR and 5′ ITR) is packaged into rAAV virions. In some embodiments, the rAAV virions comprise wild-type inverted terminal repeats, wherein the rAAV virion DNA that is generated is single-stranded (i.e., ssAAV virion). In other embodiments, a terminal resolution site in the Y ITR is deleted, resulting in formation of an rAAV virion comprising DNA that is self-complementary (i.e., scAAV virion). The scAAV forms a single-stranded DNA molecule during replication in which two single-stranded genomes comprising a plus DNA strand and a minus DNA strand are concatenated to form a self-complementary intramolecular dsDNA genome. Unlike ssAAV virions, the scAAV virions do not need to perform second-strand DNA synthesis, which increases the efficiency of scAAV transgene expression relative to ssAAV. However, the maximum cargo capacity of scAAV (i.e., maximum length of region between the 5′ ITR and 3′ ITR of the third polynucleotide construct) that can be packaged into the rAAV virion is about half that of ssAAV because the scAAV DNA packaged into a viral particle is a concatemer of two single-stranded genomes of opposite strands. For a description of methods of producing scAAV virions, see, e.g., Raj et al. (2011) Expert Rev. Hematol. 4(5):539-549, McCarty (2008) Mol. Ther. 16(10):1648-1656, McCarty et al. (2003) Gene Ther. 10(26):2112-2118; herein incorporated by reference in their entireties. For example, a ssAAV plasmid encoding a sequence of a payload can be SEQ ID NO: 39. For example, a scAAV plasmid encoding a sequence of a payload can be SEQ ID NO: 38.

### Stable Cell Lines

Various instances integrate one or more of the vectors, constructs, and/or polynucleotides described herein into the genome of a cell, cell line, and/or other collection of cells. Additional constructs may be transiently transfected into the cells to express the various constructs for rAAV expression. Transiently transfected constructs can include other vectors, constructs, and/or polynucleotides described herein and/or readily available vectors, constructs, and/or polynucleotides, including commercially available constructs, custom constructs to express the encoded components. For example, an AAV helper construct can be stably integrated in a cell, while a Rep/Cap construct and a payload construct are transiently transfected into the cell. Alternatively, an AAV helper construct and a Rep/Cap construct can be stably integrated in a cell, while a payload construct is transiently transfected; similarly an AAV helper construct and a payload construct can be stably integrated in a cell, while a Rep/Cap construct is transiently transfected. As can be appreciated, the stable cell lines in all other combinations (e.g., with 1, 2, or 3 of the AAV helper construct, Rep/Cap construct, and payload construct) can be generated in accordance with embodiments.

In some embodiments, the average concentration of Rep proteins within the cells is less than between 1-99%, 10-90%, 20-80%, 30-70%, 40-60% prior to addition of the at least first triggering agent to the cell culture medium. In some embodiments, the average concentration of Rep proteins within the cells is less than about 1%, 5%, 10%, 15%, 20%, 25%, 30%, 35%, 40%, 45%, 50%, 55%, 60%, 65%, 70%, 75%, 80%, 85%, 90%, 95%, or 99% prior to addition of the at least first triggering agent to the cell culture medium.

In some embodiments, the mammalian cell line is selected from the group consisting of a human embryonic kidney (HEK) 293 cell line, a human HeLa cell line, and a Chinese hamster ovary (CHO) cell line. In some embodiments, the mammalian cell line is a HEK293 cell line. In some embodiments, the mammalian cell line expresses adenovirus helper functions E1A and E1B.

Alternative constructs as described herein can be used in a complete system. In some embodiments, the complete system can be integrated into the host cell genome to produce a stable cell line. In other embodiments, the complete system can be transfected into the host cell and then conditional production of AAV virion from the plasmids can be induced. In some embodiments, the complete system comprises episomes in the host cell and conditional production of AAV virion from the episomes is induced.

In some embodiments, the cell is conditionally capable of producing rAAV virions having a payload encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the cell is capable of producing rAAV virions comprising the payload nucleic acid sequence at a titer of greater than 1×10¹¹ or no less than 5×10¹¹, 1×10¹², 5×10¹², 1×10¹³ or 1×10¹⁴ viral genomes per milliliter. In some embodiments, the cell is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, this cell is expanded to produce a population of cells. In some embodiments, the population of cells produces a stable cell line as described herein. In some embodiments, this cell is passaged at least three times. In some embodiments, this cell can be passaged up to 60 times. In some embodiments, this cell can be passage more than 60 times. In some embodiments, the cell maintains the ability to be conditionally induced after each passage.

### Rep/Cap Stable Cell Line

A Rep/Cap stable cell line as described herein may comprise a population of cells, wherein the population of cells comprises a cell comprising a first polynucleotide construct, which encodes for AAV Rep and Cap proteins, comprises spacer or excisable elements, and is integrated into the genome of the cell. This first polynucleotide construct (Construct 1) is also referred to as a Rep/Cap construct, and/or “AAV Rep/Cap Construct” to be used in production of rAAV virions. The Rep/Cap construct that is integrated into a cell to produce Rep/Cap stable cell line may be any first polynucleotide construct, construct 1, Rep/Cap construct, or AAV Rep/Cap Construct as described in WO2022026927, which is hereby incorporated by reference in its entirety.

In various embodiments incorporating a first polynucleotide as described herein, a second polynucleotide and a third polynucleotide can be transiently transfected. The second polynucleotide and a third polynucleotide can be the constructs such as those described herein or can be readily available (e.g., commercially available) constructs. When transfecting with a second polynucleotide and a third polynucleotide as illustrated in FIGS. 1A-1B and FIGS. 2A-2B and described herein, in such embodiments, the first and second triggering agents include doxycycline and tamoxifen, respectively. In such instances, the doxycycline induces expression of a cre recombinase (e.g., as in FIGS. 1A-1B and FIGS. 2A-2B). In the exemplary embodiments illustrated in FIGS. 2A-2B, the doxycycline further induces expression of the AAV Cap proteins. The tamoxifen allows the estrogen response element fused to the cre recombinase to translocate the cre recombinase to the nucleus to allow expression of AAV Rep proteins and possibly AAV Cap proteins (e.g., in the exemplary embodiments of FIGS. 1A-1B).

Readily available AAV helper construct may not include a cre recombinase within the construct. Under such circumstances, cre may be used as a triggering agent to induce expression of the AAV Rep proteins and possibly AAV Cap proteins (e.g., in the exemplary embodiments of FIGS. 1A-1B). In the exemplary embodiments of FIGS. 2A-2B, expression of the AAV Cap proteins is driven by an inducible promoter, thus the appropriate triggering agent (e.g., doxycycline) can be added to induce expression of the AAV Cap proteins.

### AAV Helper Stable Cell Line

An AAV helper stable cell line as described herein may comprise a population of cells, wherein the population of cells comprises a cell comprising a second polynucleotide construct, which encodes for one or more adenoviral helper proteins and is integrated into the genome of the cell. In some embodiments, the second polynucleotide encoding AAV Helper proteins is integrated into a cell to produce an AAV helper stable cell line may be any second polynucleotide construct, construct 2, or adenoviral helper construct as described herein and/or in WO2022026927, which is hereby incorporated by reference in its entirety. In some embodiments, a second polynucleotide construct integrated into the nuclear genome of the cell.

In some embodiments, in the presence of the first triggering agent, the activator activates the second inducible promoter resulting in expression of the inducible recombinase, and the inducible recombinase is expressed. In some embodiments, in the presence of a second triggering agent, the inducible recombinase translocates to a nucleus of the cell and causes recombination between the third recombination site and the fourth recombination site resulting in excision of the self-excising element, thereby operably linking the second inducible promoter to the sequence encoding the one or more adenoviral helper proteins and allowing expression of the one or more adenoviral helper proteins.

In some embodiments, in absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus.

In some embodiments where the cell comprises a second polynucleotide construct integrated into the nuclear genome of the cell, the cell comprises a transiently transfected first polynucleotide encoding AAV Rep and AAV Cap proteins and a transiently transfected third polynucleotide encoding a payload. In some embodiments, where the cell comprises a second polynucleotide construct integrated into the nuclear genome of the cell, a transiently transfected first polynucleotide encoding AAV Rep and AAV Cap proteins, and a transiently transfected third polynucleotide encoding a payload, the first polynucleotide and the third polynucleotide are not integrated into the nuclear genome of the cell.

In some embodiments, where the cell comprises a second polynucleotide construct integrated into the nuclear genome of the cell, a transiently transfected first polynucleotide encoding AAV Rep and AAV Cap proteins of FIGS. 1A-1B, and a transiently transfected third polynucleotide encoding a payload, absence of the first triggering agent and the second triggering agent, the cell expresses a fusion protein comprising a Rep protein encoded by the first part of the AAV Rep coding sequence that terminates at the stop signaling sequence. In some embodiments, in absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence.

In some embodiments, where the cell comprises a second polynucleotide construct integrated into the nuclear genome of the cell, a transiently transfected first polynucleotide encoding AAV Rep and AAV Cap proteins of FIGS. 2A-2B, and a transiently transfected third polynucleotide encoding a payload, absence of activation of the second inducible promoter by the first triggering agent, the cell does not express detectable levels of the inducible recombinase and the one or more AAV helper proteins, wherein in the absence of the second triggering agent, the inducible recombinase is unable to translocate to the nucleus. In some embodiments, absence the first triggering agent the cell does not express detectable levels of the one or more AAV capsid proteins and in the absence of the second triggering agent, the cell expresses a fusion protein comprising a Rep protein encoded by the first part of the AAV Rep coding sequence that terminates at the stop signaling sequence.

### Payload Stable Cell Line

A payload stable cell line as described herein may comprise a population of cells, wherein the population of cells comprises a cell comprising a third polynucleotide construct, which encodes for an expressible payload and is integrated into the genome of the cell. This third polynucleotide construct is also referred to as a construct 3 or payload construct to be used in production of rAAV virions. The payload construct that is integrated into a cell to produce payload stable cell line may be any third polynucleotide construct, construct 2, or payload construct as described in WO2022026927, which is hereby incorporated by reference in its entirety. In some embodiments, the payload construct encodes progranulin. In some embodiments, the payload construct encodes dystrophin. In some embodiments, the expressible payload is progranulin. In some embodiments, the expressible payload is dystrophin. In some embodiments, the dystrophin is a shortened, functional form of dystrophin. In some embodiments, a third polynucleotide construct comprises a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR). In some embodiments, the payload is progranulin or dystrophin; optionally, wherein the dystrophin is a short, functional dystrophin. In some embodiments, the third polynucleotide construct comprises at least 5%, 10%, 20%, 30%, 40%, 50%, 60%, 70%, 75%, 80%, 85%, 90%, 95%, or 100% sequence identity to SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 30; SEQ ID NO: 31; SEQ ID NO: 33; SEQ ID NO: 34; SEQ ID NO: 35; SEQ ID NO: 36; SEQ ID NO: 38; SEQ ID NO: 39; SEQ ID NO: 108; or SEQ ID NO: 109; optionally, wherein the sequence encoding progranulin is replace with a sequence encoding dystrophin; further optionally, wherein the sequence encoding dystrophin encodes for a short, functional dystrophin. In some embodiments, the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 30; SEQ ID NO: 31; SEQ ID NO: 33; SEQ ID NO: 34; SEQ ID NO: 35; SEQ ID NO: 36; SEQ ID NO: 38; SEQ ID NO: 39; SEQ ID NO: 108; or SEQ ID NO: 109; optionally, wherein the sequence encoding progranulin is replace with a sequence encoding dystrophin; further optionally, wherein the sequence encoding dystrophin encodes for a short, functional dystrophin. In some embodiments, the third polynucleotide construct further comprises a spacer between the 5′ ITR and the sequence encoding the third selectable marker or a spacer between the sequence encoding the third selectable marker and the 3′ ITR, or a combination thereof. In some embodiments, the spacer ranges in length from 500 base pairs to 5000 base pairs.

### Dual Stable Cell Line

A stable cell line may comprise a population of cells, wherein the population of cells comprises a cell comprising a first polynucleotide construct as described herein integrated into the genome of the cell and a second polynucleotide construct as described herein integrated into the genome of the cell. A stable cell line may comprise a population of cells, wherein the population of cells comprises a cell comprising a third polynucleotide construct as described herein integrated into the genome of the cell and a second polynucleotide construct as described herein integrated into the genome of the cell. a third polynucleotide construct, which encodes for an expressible payload and is integrated into the genome of the cell. A stable cell line may comprise a population of cells, wherein the population of cells comprises a cell comprising a first polynucleotide construct as described herein integrated into the genome of the cell and a third polynucleotide construct as described herein integrated into the genome of the cell.

Certain embodiments integrate two of the three constructs as described herein into the nuclear genome of a cell, such as i) integrating a first polynucleotide encoding AAV Rep/Cap proteins and a second polynucleotide encoding AAV helper proteins; ii) integrating a first polynucleotide encoding AAV Rep/Cap proteins and a third polynucleotide encoding a payload; or iii) integrating a second polynucleotide encoding AAV helper proteins and third polynucleotide encoding a payload. In some embodiments one or more other polynucleotides may be transiently transfected into the cell. In some embodiments, one or more other polynucleotides are not integrated into the nuclear genome of the cell.

### Integrated AAV Rep/Cap and AAV Helper

In some embodiments that stably integrate an AAV Rep/Cap Construct (e.g., first polynucleotide encoding AAV Rep and AAV Cap proteins) and an AAV helper construct (e.g., second polynucleotide encoding AAV helper genes) into the nuclear genome of the cell and transfecting a payload construct (e.g., third polynucleotide encoding a payload), the first polynucleotide and second polynucleotide are commensurate with FIGS. 1A-1B and FIGS. 2A-2B and the descriptions herein. In some embodiments, the third polynucleotide is transiently transfected into the cell. In some embodiments, the third polynucleotide is not integrated into the nuclear genome of the cell.

In some embodiments, the first polynucleotide comprises at least 5%, 10%, 20%, 30%, 40%, 50%, 60%, 70%, 75%, 80%, 85%, 90%, 95%, or 100% sequence identity to any one of SEQ ID NO: 1-SEQ ID NO: 4, SEQ ID NO: 24, SEQ ID NO: 99, or SEQ ID NO: 104, and the second polynucleotide comprises at least 5%, 10%, 20%, 30%, 40%, 50%, 60%, 70%, 75%, 80%, 85%, 90%, 95%, or 100% sequence identity to any one of SEQ ID NO: 5-SEQ ID NO: 13.

In some embodiments, the first polynucleotide comprises at least 5%, 10%, 20%, 30%, 40%, 50%, 60%, 70%, 75%, 80%, 85%, 90%, 95%, or 100% sequence identity to SEQ ID NO: 101; SEQ ID NO: 104; SEQ ID NO: 114, SEQ ID NO: 115; or SEQ ID NO: 114 and SEQ ID NO: 115, and the second polynucleotide comprises at least 5%, 10%, 20%, 30%, 40%, 50%, 60%, 70%, 75%, 80%, 85%, 90%, 95%, or 100% sequence identity to any one of SEQ ID NO: 5-SEQ ID NO: 13.

In some embodiments, the third polynucleotide is commensurate with the payload constructs described herein or may be readily available payload constructs. In some embodiments, the third polynucleotide comprises at least 5%, 10%, 20%, 30%, 40%, 50%, 60%, 70%, 75%, 80%, 85%, 90%, 95%, or 100% sequence identity to SEQ ID NO: 15; SEQ ID NO: 17; SEQ ID NO: 19; SEQ ID NO: 21; SEQ ID NO: 108; or SEQ ID NO: 109. In some embodiments, the sequence encoding progranulin is replaced with a sequence encoding dystrophin. In some embodiments, the sequence encoding dystrophin encodes for a short, functional dystrophin.

In such embodiments, the first and second triggering agents include doxycycline and tamoxifen, respectively. In such instances, the doxycycline induces expression of a cre recombinase (e.g., as in FIGS. 1A-1B and FIGS. 2A-2B). In the exemplary embodiments illustrated in FIGS. 2A-2B, the doxycycline further induces expression of the AAV Cap proteins. The tamoxifen allows the estrogen response element fused to the cre recombinase to translocate the cre recombinase to the nucleus to allow expression of AAV Rep proteins and possibly AAV Cap proteins (e.g., in the exemplary embodiments of FIGS. 1A-1B).

In some embodiments (e.g., when the first polynucleotide encoding AAV Rep and Cap proteins is akin to the exemplary embodiments of FIGS. 1A-1B), absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence.

In some embodiments (e.g., when the first polynucleotide encoding AAV Rep and Cap proteins is akin to the exemplary embodiments of FIGS. 2A-2B), absence of activation of the second inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent the cell does not express detectable levels of the one or more AAV capsid proteins and in the absence of the second triggering agent, the cell expresses a fusion protein comprising a Rep protein encoded by the first part of the AAV Rep coding sequence that terminates at the stop signaling sequence.

### Integrated AAV Rep/Cap and Payload

In some embodiments that stably integrate a first polynucleotide (i.e., first polynucleotide encoding AAV Rep and/or Cap proteins) and a third polynucleotide (i.e., third polynucleotide encoding a payload) and transfecting an AAV helper construct (e.g., Construct 2), the AAV Rep/Cap Construct (e.g., Construct 1) and AAV helper construct (e.g., Construct 2) are commensurate with FIGS. 1A-1B and FIGS. 2A-2B and the descriptions herein. In some embodiments, the AAV Rep/Cap Construct comprises at least 5%, 10%, 20%, 30%, 40%, 50%, 60%, 70%, 75%, 80%, 85%, 90%, 95%, or 100% sequence identity to any one of SEQ ID NO: 1-SEQ ID NO: 4, SEQ ID NO: 22, SEQ ID NO: 23, SEQ ID NO: 99, SEQ ID NO: 101, or SEQ ID NO: 102, and the payload construct may be commensurate with the plasmids described herein. The AAV helper construct can be a readily available construct (e.g., commercially available) or it may be commensurate with an inducible AAV helper construct as illustrated in FIGS. 1A-1B and FIGS. 2A-2B and described herein.

In embodiments that transfect with an AAV helper construct commensurate with the constructs described herein, the AAV helper construct comprises at least 5%, 10%, 20%, 30%, 40%, 50%, 60%, 70%, 75%, 80%, 85%, 90%, 95%, or 100% sequence identity to any one of SEQ ID NO: 5-SEQ ID NO: 13. In such embodiments, the first and second triggering agents include doxycycline and tamoxifen, respectively. In such instances, the doxycycline induces expression of a cre recombinase (e.g., as in FIGS. 1A-1B and FIGS. 2A-2B). In the exemplary embodiments illustrated in FIGS. 2A-2B, the doxycycline further induces expression of the AAV Cap proteins. The tamoxifen allows the estrogen response element fused to the cre recombinase to translocate the cre recombinase to the nucleus to allow expression of AAV Rep proteins and possibly AAV Cap proteins (e.g., in the exemplary embodiments of FIGS. 1A-1B).

Readily available AAV helper construct may not include a cre recombinase within the construct. Under such circumstances, cre may be used as a triggering agent to induce expression of the AAV Rep proteins and possibly AAV Cap proteins (e.g., in the exemplary embodiments of FIGS. 1A-1B). In the exemplary embodiments of FIGS. 2A-2B, expression of the AAV Cap proteins is driven by an inducible promoter, thus the appropriate triggering agent (e.g., doxycycline) can be added to induce expression of the AAV Cap proteins.

### Integrated AAV Helper and Payload

A third combination of stably integrated constructs in accordance with some embodiments comprises a cell comprising an second polynucleotide encoding AAV helper proteins and a third polynucleotide encoding a payload integrated into the nuclear genome of the cell. In some embodiments, the AAV helper construct and payload construct are commensurate with FIGS. 1A-1B and FIGS. 2A-2B and the descriptions herein. In some such embodiments, the second polynucleotide and the third polynucleotide payload construct are commensurate with the plasmids and nucleic acid constructs described herein. The transfected AAV Rep/Cap construct can be a readily or commercially available construct or it may be commensurate with AAV Rep/Cap constructs as described herein, including FIGS. 1A-1B and FIGS. 2A-2B. In such embodiments, the first and second triggering agents include doxycycline and tamoxifen, respectively. In such instances, the doxycycline induces expression of a cre recombinase (e.g., as in FIGS. 1A-1B and FIGS. 2A-2B). In the exemplary embodiments illustrated in FIGS. 2A-2B, the doxycycline further induces expression of the AAV Cap proteins. The tamoxifen allows the estrogen response element fused to the cre recombinase to translocate the cre recombinase to the nucleus to allow expression of AAV Rep proteins and possibly AAV Cap proteins (e.g., in the exemplary embodiments of FIGS. 1A-1B).

In some embodiments, where the non-integrated and/or transiently transfected first polynucleotide is commensurate with the exemplary embodiments of FIGS. 1A-1B, absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence.

In some embodiments, where the non-integrated and/or transiently transfected first polynucleotide is commensurate with the exemplary embodiments of FIGS. 2A-2B, absence of activation of the second inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent the cell does not express detectable levels of the one or more AAV capsid proteins and in the absence of the second triggering agent, the cell expresses a fusion protein comprising a Rep protein encoded by the first part of the AAV Rep coding sequence that terminates at the stop signaling sequence.

Methods of Producing rAAV

Provided herein are methods of using the stable cell lines to produce recombinant AAV (rAAV) virions within which are packaged an expressible payload. In some embodiments, the stable cell line is a stable cell line that is transiently transfected with one or more plasmids, wherein these cells are capable of conditionally producing recombinant AAV (rAAV) virions within which are packaged an expressible payload; and production of virions is inducible upon addition of a triggering agent. In some embodiments, the stable cell line is a stable AAV helper cell line that is transiently transfected with a Rep/Cap plasmid and a payload plasmid. In some embodiments, the stable cell line is a stable Rep/Cap cell line that is transiently transfected with a AAV helper plasmid and a payload plasmid. In some embodiments, the stable cell line is a payload stable cell line that is transiently transfected with a Rep/Cap plasmid and an AAV helper plasmid. In some embodiments, the triggering agent is doxycycline. In some embodiments, the triggering agent is tamoxifen. In some embodiments, a first triggering agent and a second triggering agent induce virion production. In some embodiments, the first triggering agent is doxycycline and the second triggering agent is tamoxifen.

Methods for Producing rAAV Using Rep/Cap Stable Cell Line

A Rep/Cap stable line as disclosed herein may be transiently transfected with one or more plasmids to produce cells are capable of conditionally producing recombinant AAV (rAAV) virions within which are packaged an expressible payload.

### Dual Transient Transfection

A Rep/Cap stable line as disclosed herein may be transiently transfected with one or more plasmids to produce cells are capable of conditionally producing recombinant AAV (rAAV) virions within which are packaged an expressible payload. The Rep/Cap stable cell line may comprise cells that stably integrate the first polynucleotide construct as disclosed herein. The one or more plasmids may not integrate into the genomes of the cells. The one or more plasmids may be an AAV helper plasmid. An AAV helper plasmid may be any commercially available AAV helper plasmid. The AAV helper plasmid may be a plasmid comprising the second polynucleotide construct as described herein. The one or more plasmids may be a payload plasmid. The payload plasmid may be any plasmid comprising the payload flanked by ITRs. The payload plasmid may be a plasmid comprising the third polynucleotide construct as described herein. The one or more plasmids may be an AAV helper plasmid and a payload plasmid.

### Single Transient Transfection

A Rep/Cap stable line as disclosed herein may be transiently transfected with one or more plasmids to produce cells are capable of conditionally producing recombinant AAV (rAAV) virions within which are packaged an expressible payload. The Rep/Cap stable cell line may comprise cells that stably integrate the first polynucleotide construct as disclosed herein. In some embodiments, the Rep/Cap stable cell line my further comprise cells that stably integrate the second polynucleotide construct as disclosed herein. The one or more plasmids may not integrate into the genomes of the cells. The one or more plasmids may be a payload plasmid. The payload plasmid may be any plasmid comprising the payload flanked by ITRs. The payload plasmid may be a plasmid comprising the third polynucleotide construct as described herein.

A Rep/Cap stable line as disclosed herein may be transiently transfected with one or more plasmids to produce cells are capable of conditionally producing recombinant AAV (rAAV) virions within which are packaged an expressible payload. The Rep/Cap stable cell line may comprise cells that stably integrate the first polynucleotide construct as disclosed herein. In some embodiments, the Rep/Cap stable cell line my further comprise cells that stably integrate the third polynucleotide construct as disclosed herein. The one or more plasmids may not integrate into the genomes of the cells. The one or more plasmids may be an AAV helper plasmid. An AAV helper plasmid may be any commercially available Rep/Cap plasmid. The AAV helper plasmid may be a plasmid comprising the second polynucleotide construct as described herein.

Methods for Producing rAAV Using a AAV Helper Stable Cell Line

An AAV helper stable line as disclosed herein may be transiently transfected with one or more plasmids to produce cells are capable of conditionally producing recombinant AAV (rAAV) virions within which are packaged an expressible payload.

### Dual Transient Transfection

An AAV helper stable line as disclosed herein may be transiently transfected with one or more plasmids to produce cells are capable of conditionally producing recombinant AAV (rAAV) virions within which are packaged an expressible payload. The AAV helper stable cell line may comprise cells that stably integrate the second polynucleotide construct as disclosed herein. The one or more plasmids may not integrate into the genomes of the cells. The one or more plasmids may be a Rep/Cap plasmid. A Rep/Cap plasmid may be any commercially available Rep/Cap plasmid. The Rep/Cap plasmid may be a plasmid comprising the first polynucleotide construct as described herein. The one or more plasmids may be a payload plasmid. The payload plasmid may be any plasmid comprising the payload flanked by ITRs. The payload plasmid may be a plasmid comprising the third polynucleotide construct as described herein. The one or more plasmids may be a Rep/Cap plasmid and a payload plasmid.

In some aspects, a system for inducibly producing recombinant adenovirus associated virus (rAAV) virions comprises: (a) a first plasmid according to any one of the embodiments disclosed herein; (b) a cell comprising the second polynucleotide construct integrated into the nuclear genome of the cell according to any one of the embodiments disclosed herein; and (c) a second plasmid according to any one of the embodiments disclosed herein; optionally further comprising (d) a fourth polynucleotide construct according to any one of the embodiments disclosed herein; and further optionally comprising a fifth polynucleotide construct according to any one of the embodiments disclosed herein.

In some aspects, a system for inducibly producing recombinant adenovirus associated virus (rAAV) virions comprises: (a) the first polynucleotide construct in the first plasmid according to any one of the embodiments disclosed herein; (b) a cell comprising the second polynucleotide construct integrated into the nuclear genome of the cell according to any one of the embodiments disclosed herein; and (c) the third polynucleotide construct in the second plasmid according to any one of the embodiments disclosed herein; optionally further comprising (d) a fourth polynucleotide construct according to any one of the embodiments disclosed herein; and further optionally comprising a fifth polynucleotide construct according to any one of the embodiments disclosed herein. In some embodiments, the system further comprises the first triggering agent; optionally, wherein the first triggering agent is doxycycline. In some embodiments, the system further comprises the second triggering agent; optionally, wherein the second triggering agent is tamoxifen.

In some aspects, a method of generating a cell for inducibly producing recombinant AAV (rAAV) virions comprising a payload comprises: introducing into a cell a second polynucleotide construct according to any one of the embodiments disclosed herein; selecting for cells expressing the second selectable marker; introducing into a cell of the cells expressing the second selectable marker the first polynucleotide construct according to any one of the embodiments disclosed herein and the third polynucleotide construct according to any one of the embodiments disclosed herein, optionally, wherein the introducing is via transient transfection; thereby generating the cell inducibly producing recombinant AAV (rAAV) virions comprising a payload wherein the first polynucleotide construct is integrated into the nuclear genome of the cell and the first polynucleotide construct and the third polynucleotide construct are not integrated into the genome of the cell.

In some aspects, a method of generating a cell for inducibly producing recombinant AAV (rAAV) virions comprising a payload comprises: introducing into a cell a second polynucleotide construct according to any one of the embodiments disclosed herein; selecting for cells expressing the second selectable marker; introducing into a cell of the cells expressing the second selectable marker the first plasmid according to any one of the embodiments disclosed herein and the second plasmid according to any one of claims the embodiments disclosed herein, optionally, wherein the introducing is via transient transfection; thereby generating the cell inducibly producing recombinant AAV (rAAV) virions comprising a payload wherein the first polynucleotide construct is integrated into the nuclear genome of the cell and the first plasmid and the plasmid are not integrated into the genome of the cell. In some embodiments, the method further comprising contacting the cell for inducibly producing recombinant AAV (rAAV) virions comprising a payload to the first triggering agent and the second triggering agent, wherein in the presence of the first triggering agent, the activator activates the inducible promoter resulting in expression of the inducible recombinase, wherein in the presence of the second triggering agent, the inducible recombinase translocates to the nucleus, wherein recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; thereby inducibly producing recombinant AAV (rAAV) virions comprising a payload; optionally, further comprising (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of AAV Rep proteins and AAV Cap proteins, wherein the one or more promoters are operably linked to the complete AAV Rep coding sequence to allow expression of AAV Rep proteins and AAV Cap proteins. In some embodiments, the cell is a mammalian cell. In some embodiments, the mammalian cell is a HEK293 cell. In some embodiments, the first polynucleotide construct is in a plasmid comprising at least 70%, 75%, 80%, 85%, 90%, 95%, or 100% sequence identity to SEQ ID NO: 3. In some embodiments, the second polynucleotide construct is in a plasmid comprising at least 70%, 75%, 80%, 85%, 90%, 95%, or 100% sequence identity to SEQ ID NO: 12. In some embodiments, the third polynucleotide construct is in a plasmid comprising at least 70%, 75%, 80%, 85%, 90%, 95%, or 100% sequence identity to SEQ ID NO: 15; optionally, wherein the sequence encoding progranulin is replaced with a sequence encoding dystrophin; further optionally, wherein the sequence encoding dystrophin encodes for a short, functional dystrophin. In some embodiments, the third polynucleotide construct is in a plasmid comprising at least 70%, 75%, 80%, 85%, 90%, 95%, or 100% sequence identity to SEQ ID NO: 17; optionally, wherein the sequence encoding progranulin is replaced with a sequence encoding dystrophin; further optionally, wherein the sequence encoding dystrophin encodes for a short, functional dystrophin. In some embodiments, the third polynucleotide construct is in a plasmid comprising at least 70%, 75%, 80%, 85%, 90%, 95%, or 100% sequence identity to SEQ ID NO: 19; optionally, wherein the sequence encoding progranulin is replaced with a sequence encoding dystrophin; further optionally, wherein the sequence encoding dystrophin encodes for a short, functional dystrophin. In some embodiments, the third polynucleotide construct is in a plasmid comprising at least 70%, 75%, 80%, 85%, 90%, 95%, or 100% sequence identity to SEQ ID NO: 21.

In some aspects, a method for generating a recombinant adenovirus associated virus (rAAV) virion comprising a sequence encoding a payload comprises contacting the cell according to any one of the embodiments disclosed herein with the first triggering agent and the second triggering agent, wherein in the presence of the first triggering agent, the activator activates the inducible promoter of the second polynucleotide construct resulting in expression of the inducible recombinase, wherein in the presence of the second triggering agent, the inducible recombinase translocates to the nucleus, wherein recombination between the first recombination site and the second recombination site in the first polynucleotide construct by the inducible recombinase results in excision of the excisable element or inversion of the inversible element, wherein the inducible promoter becomes operably linked to the sequence encoding the one or more AAV helper proteins to allow expression of the one or more AAV helper proteins; and optionally, the first part of the AAV Rep coding sequence and the second part of the AAV Rep coding sequence are joined to form a complete AAV Rep coding sequence, wherein the one or more promoters are operably linked to the complete AAV Rep coding sequence to allow expression of AAV Rep proteins and AAV Cap proteins, and recombination between the third recombination site and the fourth recombination site in the second polynucleotide construct by the inducible recombinase results in excision of the self-excising element comprising the sequence encoding the inducible recombinase, wherein the expression of the one or more AAV helper proteins results in expression of the one or more Rep proteins and the one or more capsid proteins, thereby generating an rAAV virion comprising the sequence encoding the payload of interest. In some embodiments, the payload is progranulin or dystrophin; optionally, wherein the dystrophin is a short, functional dystrophin. In some embodiments, the first triggering agent is doxycycline. In some embodiments, the second triggering agent is tamoxifen.

### Single Transient Transfection

An AAV helper stable line as disclosed herein may be transiently transfected with one or more plasmids to produce cells are capable of conditionally producing recombinant AAV (rAAV) virions within which are packaged an expressible payload. The AAV helper stable cell line may comprise cells that stably integrate the second polynucleotide construct as disclosed herein. In some embodiments, the AAV helper stable cell line my further comprise cells that stably integrate the first polynucleotide construct as disclosed herein. The one or more plasmids may not integrate into the genomes of the cells. The one or more plasmids may be a payload plasmid. The payload plasmid may be any plasmid comprising the payload flanked by ITRs. The payload plasmid may be a plasmid comprising the third polynucleotide construct as described herein.

An AAV helper stable line as disclosed herein may be transiently transfected with one or more plasmids to produce cells are capable of conditionally producing recombinant AAV (rAAV) virions within which are packaged an expressible payload. The AAV helper stable cell line may comprise cells that stably integrate the second polynucleotide construct as disclosed herein. In some embodiments, the AAV helper stable cell line my further comprise cells that stably integrate the third polynucleotide construct as disclosed herein. The one or more plasmids may not integrate into the genomes of the cells. The one or more plasmids may be a Rep/Cap plasmid. A Rep/Cap plasmid may be any commercially available Rep/Cap plasmid. The Rep/Cap plasmid may be a plasmid comprising the first polynucleotide construct as described herein.

Methods for Producing rAAV Using Payload Stable Cell Line

A payload stable line as disclosed herein may be transiently transfected with one or more plasmids to produce cells are capable of conditionally producing recombinant AAV (rAAV) virions within which are packaged an expressible payload.

### Dual Transient Transfection

A payload stable line as disclosed herein may be transiently transfected with one or more plasmids to produce cells are capable of conditionally producing recombinant AAV (rAAV) virions within which are packaged an expressible payload. The payload stable cell line may comprise cells that stably integrate the third polynucleotide construct as disclosed herein. The one or more plasmids may not integrate into the genomes of the cells. The one or more plasmids may be a Rep/Cap plasmid. A Rep/Cap plasmid may be any commercially available Rep/Cap plasmid. The Rep/Cap plasmid may be a plasmid comprising the first polynucleotide construct as described herein. The one or more plasmids may be an AAV helper plasmid. An AAV helper plasmid may be any commercially available AAV helper plasmid. The AAV helper plasmid may be a plasmid comprising the second polynucleotide construct as described herein. The one or more plasmids may be a Rep/Cap plasmid and an AAV helper plasmid.

### Single Transient Transfection

A payload stable line as disclosed herein may be transiently transfected with one or more plasmids to produce cells are capable of conditionally producing recombinant AAV (rAAV) virions within which are packaged an expressible payload. The payload stable cell line may comprise cells that stably integrate the third polynucleotide construct as disclosed herein. In some embodiments, the payload stable cell line my further comprise cells that stably integrate the first polynucleotide construct as disclosed herein. The one or more plasmids may be an AAV helper plasmid. An AAV helper plasmid may be any commercially available AAV helper plasmid. The AAV helper plasmid may be a plasmid comprising the second polynucleotide construct as described herein. The one or more plasmids may be a Rep/Cap plasmid and an AAV helper plasmid.

A payload stable line as disclosed herein may be transiently transfected with one or more plasmids to produce cells are capable of conditionally producing recombinant AAV (rAAV) virions within which are packaged an expressible payload. The payload stable cell line may comprise cells that stably integrate the third polynucleotide construct as disclosed herein. In some embodiments, the AAV helper stable cell line my further comprise cells that stably integrate the second polynucleotide construct as disclosed herein. The one or more plasmids may not integrate into the genomes of the cells. The one or more plasmids may be a Rep/Cap plasmid. A Rep/Cap plasmid may be any commercially available Rep/Cap plasmid. The Rep/Cap plasmid may be a plasmid comprising the first polynucleotide construct as described herein.

Methods for Producing rAAV Using Triple Transient Transfection

A population of cells may be transiently transfected with one or more plasmids to produce cells are capable of conditionally producing recombinant AAV (rAAV) virions within which are packaged an expressible payload. In some embodiments, the transient transfection is triple transient transfection. The one or more plasmids for triple transient transfection may not integrate into the genomes of the cells. The one or more plasmids for triple transient transfection may be a Rep/Cap plasmid. A Rep/Cap plasmid may be any commercially available Rep/Cap plasmid. The Rep/Cap plasmid may be a plasmid comprising the first polynucleotide construct as described herein. The one or more plasmids for triple transient transfection may be an AAV helper plasmid. An AAV helper plasmid may be any commercially available AAV helper plasmid. The AAV helper plasmid may be a plasmid comprising the second polynucleotide construct as described herein. The one or more plasmids for triple transient transfection may be a payload plasmid. The payload plasmid may be any plasmid comprising the payload flanked by ITRs. The payload plasmid may be a plasmid comprising the third polynucleotide construct as described herein. The one or more plasmids for triple transient transfection may be a Rep/Cap plasmid, an AAV helper plasmid, and a payload plasmid.

rAAV Virion Produced by Methods of the Present Disclosure

In some aspects, the rAAV virion produced by the methods described in section “Methods of producing rAV.”

### Pharmaceutical Compositions

The present disclosure provides pharmaceutical compositions comprising the polynucleotides, or vector system described herein or an rAAV virion encapsidating a polynucleotide payload (e.g., for encoding a therapeutic protein, such as an antibody or any fragment or derivative thereof), produced from such a vector system, and a pharmaceutically acceptable carrier, diluent, excipient, or buffer. In some cases, the pharmaceutically acceptable carrier, diluent, excipient, or buffer is suitable for use in a human. Such excipients, carriers, diluents, and buffers include any pharmaceutical agent that may be administered without undue toxicity.

Pharmaceutically acceptable excipients include, but are not limited to, liquids such as water, saline, glycerol, polyethylene glycol, hyaluronic acid, and ethanol. Pharmaceutically acceptable salts may be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H.C. Ansel et al., eds., 7^{th} ed., Lippincott, Williams, & Wilkins; and Handbook of Pharmaceutical Excipients (2000) A.H. Kibbe et al., eds., 3^{rd} ed. Amer. Pharmaceutical Assoc. Certain facilitators of nucleic acid uptake and/or expression may also be included in the compositions or coadministered.

In some embodiments, a pharmaceutical composition comprises the plurality of rAAV virions of any one of the embodiments as disclosed herein as disclosed herein and a pharmaceutically acceptable carrier. In some embodiment, a plurality of pharmaceutical doses each independently comprise the plurality of rAAV virions of any one of the embodiments as disclosed herein as disclosed herein and a pharmaceutically acceptable carrier. In some embodiments, the encapsidation ratio has a difference of not more than 20%, 10%, 5%, 4%, 3%, 2%, or 1% between a first dose and a second dose of a plurality of pharmaceutical doses. In some embodiments, the F:E ratio has a difference of not more than 20%, 10%, 5%, 4%, 3%, 2%, or 1% between a first dose and a second dose of a plurality of pharmaceutical doses. In some embodiments, the concentration of viral genomes has a difference of not more than 20%, 10%, 5%, 4%, 3%, 2%, or 1% between a first dose and a second dose of a plurality of pharmaceutical doses. In some embodiments, the concentration of vector genomes has a difference of not more than 20%, 10%, 5%, 4%, 3%, 2%, or 1% between a first dose and a second dose of a plurality of pharmaceutical doses. In some embodiments, the rAAV virion infectivity has a difference of not more than 20%, 10%, 5%, 4%, 3%, 2%, or 1% between a first dose and a second dose of a plurality of pharmaceutical doses.

### Methods of Treatment

In another aspect, methods of treatment are provided. In various embodiments, the method comprises administering rAAV produced by the process described above to a patient in need thereof. In some embodiments, the administering is by intravenous administration, intramuscular administration, intrathecal administration, intracisternal administration, or administration via brain surgery.

In some embodiments, a method of treating a condition or disorder comprises administering a therapeutically effective amount of the pharmaceutical composition of as disclosed herein to a patient in need thereof. In some embodiments, the disorder is a monogenic disorder. In some embodiments, the treatment results in at least one undesirable side effect and wherein the undesirable side effect is reduced relative to administering a daily dose that deviates more than 50%, 40%, 30%, 30%, 15%, 10%, 5%, or 2% from an expected dose. In some embodiments, the administering is by injection. In some embodiments, the injection is an infusion. In some embodiments, the daily dose is administered to the patient once. In some embodiments, the daily dose is administered to the patient two or more times. In some embodiments, the treatment results in at least one undesirable side effect and wherein the undesirable side effect is reduced relative to administering a plurality of rAAV virions produced from a triple transfection method.

In some embodiments, the methods reduce the immunogenicity of a dose of rAAV having a predetermined number of viral genomes (VG) as compared to the same rAAV VG dose prepared by transient triple transfection. In some embodiments, the immunogenicity is measured by the titer or concentration of neutralizing antibodies in a subject. In some embodiments, a concentration of rAAV virion neutralizing antibody in the blood serum of the patient is reduced relative to a concentration of rAAV virion neutralizing antibody in the blood serum of a patient after administering a plurality of rAAV virions produced from a triple transfection method. In some embodiments, the concentration of rAAV virion neutralizing antibodies is measured by an ELISA assay.

In some embodiments, the methods reduce the number or intensity of adverse effects caused by administering a dose of rAAV having a predetermined number of viral genomes (VG) as compared to the same rAAV VG dose prepared by transient triple transfection. In some embodiments, the methods reduce the number of adverse effects. In some embodiments, the predetermined number of VG in a dose is no greater than 3×10¹⁴ vg/kg. In some embodiments, the predetermined number of VG in a dose is no greater than 1×10¹⁴ vg/kg. In some embodiments, the predetermined number of VG in a dose is no greater than 5×10¹³ vg/kg. In some embodiments, the methods reduce the intensity of adverse effects. In some embodiments, the methods reduce both the number and the intensity of adverse events.

In some embodiments, a method of administering a dose of rAAV virions having a predetermined number of viral genomes (VG) to a subject with reduced number or intensity of adverse effects as compared to administration of the same rAAV VG dose prepared by transient triple transfection comprises: administering a dose of rAAV produced in the cell as disclosed herein, the population of cells disclosed herein, or the stable cells as disclosed herein. In some embodiments, the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death. In some embodiments, the adverse effect is an increase in serum levels of one or more proinflammatory cytokines. In some embodiments, the adverse effect is an increase in serum levels of one or more of interferon gamma (IFNγ), interleukin 1β (IL-1β), and interleukin 6 (IL-6).

In another aspects, a method of repeatedly administering a dose of rAAV to a subject in need thereof is provided. In some embodiments, the method comprises administering a first dose of rAAV produced by the cell lines and the processes described above, and then administering at least a second dose of rAAV produced by the cell lines and the processes described above. In some embodiments, the method comprises administering a first dose and a second dose of rAAV produced by the cell lines and the processes described above. In some embodiments, the method comprises administering a first dose, a second dose, and a third dose of rAAV produced by the cell lines and the processes described above. In some embodiments, the method comprises administering more than three doses of rAAV produced by the cell lines and the processes described above. In some embodiments, the first dose of rAAV and the at least second dose of rAAV are administered through the same route of administration. In some embodiments, the first dose of rAAV and the at least second dose of rAAV are administered through different routes of administration. In some embodiments, the route of administration is intravenous administration, intramuscular administration, intrathecal administration, intracisternal administration, or administration via brain surgery.

In some embodiments, a method of treating a condition or disorder comprises administering a first therapeutically effective amount of the pharmaceutical composition of as disclosed herein having a predetermined number of viral genomes to a patient in need thereof and a second therapeutically effective amount of the pharmaceutical composition as disclosed herein having the predetermined number of viral genomes to the patient in need thereof. In some embodiments, the first therapeutically effective amount and the second therapeutically effective amount vary by no more than 1%, 5%, 10%, or 15%.

### Methods of Delivering a Payload or Protein

Once formulated, compositions comprising an rAAV virion or protein (e.g., therapeutic protein such as an antibody or any fragment or derivative thereof) may be administered directly to a subject or, alternatively, delivered ex vivo, to cells derived from the subject. For example, methods for the ex vivo delivery and reimplantation of transformed cells into a subject are known in the art and may include, e.g., dextran-mediated transfection, calcium phosphate precipitation, polybrene mediated transfection, lipofectamine and LT-1 mediated transfection, protoplast fusion, electroporation, encapsulation of the polynucleotide(s) in liposomes, and direct microinjection of the DNA into nuclei. Direct delivery of a vector system comprising an expressible sequence encoding a payload of interest in vivo will generally be accomplished by injection using either a conventional syringe, needless devices such as Bioject or a gene gun, such as the Accell gene delivery system (PowderMed Ltd, Oxford, England).

In certain embodiments, rAAV of the present disclosure or compositions comprising the rAAV may be administered to a subject in need thereof by any suitable route, such as, intravenous, intramuscular, intracranial, intracerebroventicular, intrathecal, intracisternal, or via brain surgery.

In some embodiments, the rAAV virions comprising an expressible sequence encoding a payload of interest are used in gene therapy applications to treat a disease. The payload may be, for example, a polypeptide, a protein, or an RNA. A polypeptide or a protein may be, for example, an enzyme, an antibody, a hormone, an aptamer, or an endonuclease (e.g., a site-specific endonuclease such an RNA-guided endonuclease), a component of a CRISPR/Cas system, an adenosine deaminase acting on RNA (ADAR) enzyme, a transcriptional activator, a transcriptional repressor, or any combination thereof, as described above. The payload may be progranulin. An RNA may be, for example, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof. In some embodiments, the rAAV virions used in gene therapy applications to treat a disease comprise one or more expressible sequences encoding one or more payloads of interest. For example, the rAAV virions comprise two expressible sequences, wherein a first expressible sequence encodes for a first gRNA and a second expressible sequence encodes for a second gRNA. In some embodiments, the first gRNA and the second gRNA are different. In some embodiments, the first gRNA and the second gRNA are the same.

In some embodiments, the protein (e.g., therapeutic protein such as an antibody or any fragment or derivative thereof) is used in gene therapy applications to treat a disease. The protein may be, for example, a polypeptide. A polypeptide or a protein may be, for example, an enzyme, an antibody, a hormone, an aptamer, or an endonuclease (e.g., a site-specific endonuclease such an RNA-guided endonuclease), a component of a CRISPR/Cas system, an adenosine deaminase acting on RNA (ADAR) enzyme, a transcriptional activator, a transcriptional repressor, or any combination thereof, as described above.

The rAAV virions or protein (e.g., therapeutic protein) may be formulated into compositions for delivery to a vertebrate subject (e.g., mammalian subject, preferably human). These compositions may either be prophylactic (to prevent a disease or condition) or therapeutic (to treat a disease or condition). The compositions will comprise a “therapeutically effective amount” of the rAAV virions such that amounts of the payload of interest may be produced in vivo sufficient to have a therapeutic benefit in the individual to which it is administered. The compositions will comprise a “therapeutically effective amount” of the protein (e.g., therapeutic protein) such that amounts have a therapeutic benefit in the individual to which it is administered. The exact amounts necessary will vary depending on the subject being treated; the age and general condition of the subject to be treated; the degree of protection desired; the severity of the condition being treated; the particular therapeutic agent produced, and the mode of administration, among other factors. An appropriate effective amount may be readily determined by one of skill in the art. Thus, a “therapeutically effective amount” will fall in a relatively broad range that may be determined through routine trials.

A “therapeutically effective amount” of virion comprising an expressible sequence encoding a payload of interest will fall in a relatively broad range that may be determined through experimentation and/or clinical trials. For example, for in vivo injection, a therapeutically effective dose of rAAV virions will be on the order of from about 10⁶ to about 10¹⁵ of the rAAV virions, e.g., from about 10⁸ to 10¹² rAAV virions. For in vitro transduction, an effective amount of rAAV virions to be delivered to cells will be on the order of from about 10⁸ to about 10¹³ of the rAAV virions. Other effective dosages may be readily established by one of ordinary skill in the art through routine trials establishing dose response curves.

In some cases, more than one administration (e.g., two, three, four or more administrations) may be employed to achieve the desired level of gene expression. In some cases, more than one administration is administered at various intervals, e.g., daily, weekly, twice monthly, monthly, every 3 months, every 6 months, yearly, etc. In some cases, multiple administrations are administered over a period of time from 1 month to 2 months, from 2 months to 4 months, from 4 months to 8 months, from 8 months to 12 months, from 1 year to 2 years, from 2 years to 5 years, or more than 5 years.

### Kits

In another aspect, components or embodiments described herein for the system are provided in a kit. For example, any of the plasmids, as well as the mammalian cells, related buffers, media, triggering agents, or other components related to cell culture and virion production can be provided, with optional components frozen and packaged as a kit, alone or along with separate containers of any of the other agents and optional instructions for use. In some embodiments, the kit may comprise culture vessels, vials, tubes, or the like.

The methods for producing and packaging recombinant vectors in desired AAV capsids to produce the rAAVs are not meant to be limiting and other suitable methods will be apparent to the skilled artisan.

### ASPECTS OF THE INVENTION

The below items disclose various aspects of the invention. Each of the aspects described below can be combined with other aspects and embodiments disclosed elsewhere herein, including the claims, where the combinations are clearly compatible. Certain aspects include:

Aspect 1. A cell for inducibly producing recombinant adenovirus associated virus (rAAV) virions, the cell comprising:
(a) a first plasmid comprising a first polynucleotide construct comprising an AAV Rep coding sequence, a sequence encoding one or more AAV capsid proteins, and a first constitutive promoter operably linked to a sequence encoding a first selectable marker, optionally, wherein the first polynucleotide construct comprises from 5′ to 3′:
one or more promoters operably linked to a first sequence comprising a first part of the AAV Rep coding sequence;
a second sequence comprising a second part of the AAV Rep coding sequence, wherein the one or more promoters are not operably linked to the second sequence comprising the second part of the AAV Rep coding sequence,
wherein the first sequence and the second sequence are separated by (i) an excisable element that comprises a first recombination site and a second recombination site flanking a coding sequence encoding a stop signaling sequence, wherein the first recombination site and the second recombination site are oriented in the same direction or (ii) an inversible element that comprises a first recombination site and a second recombination site flanking a coding sequence encoding a stop signaling sequence, wherein the first recombination site and the second recombination site are oriented in opposite directions; and
a third sequence comprising the sequence encoding one or more AAV capsid proteins, wherein the second sequence comprises a promoter that is operably linked to the third sequence; and the first constitutive promoter operably linked to a sequence encoding the first selectable marker; (b) a second polynucleotide construct integrated into the nuclear genome of the cell, comprising from 5′ to 3′:
a second constitutive promoter operably linked to a sequence encoding an activator, wherein the cell constitutively expresses the activator and the activator is unable to activate an inducible promoter in absence of a first triggering agent;
the inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; a sequence encoding one or more AAV helper proteins, wherein the inducible promoter is not operably linked to the sequence encoding the one or more AAV helper proteins; and a third constitutive promoter operably linked to a sequence encoding a second selectable marker, wherein the cell constitutively expresses the second selectable marker; and
(c) a second plasmid comprising a third polynucleotide construct comprising a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR);
wherein in absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence.
   Aspect 2. The cell of Aspect 1, wherein the payload is progranulin or dystrophin; optionally, wherein the dystrophin is a short, functional dystrophin.
   Aspect 3. The cell of Aspect 1 or 2, wherein the coding sequence encoding the stop signaling sequence further encodes a protein marker that comprises the stop signaling sequence.
   Aspect 4. The cell of Aspect any one of Aspects 1-3, wherein the cell further comprises: an adenovirus E1A protein and E1B protein, and the one or more AAV helper proteins expressed by the second polynucleotide construct are an adenovirus E2A protein and E4 protein, or an adenovirus E2A protein and E4 protein, and the one or more AAV helper proteins expressed by the second polynucleotide construct are an adenovirus E1A protein and E1B protein.
   Aspect 5. The cell of any one of Aspects 1-4, further comprising a fourth polynucleotide construct comprising an inducible or constitutive promoter operably linked to a sequence encoding one or more helper proteins.
   Aspect 6. The cell of any one of Aspects 1-5, wherein the sequence coding for one or more AAV helper proteins comprises a bicistronic open reading frame encoding two AAV helper proteins.
   Aspect 7. The cell of Aspect 6, wherein the two AAV helper proteins are E2A and E4 or E1A and E1B.
   Aspect 8. The cell of Aspect 6 or 7, wherein the bicistronic open reading frame comprises an internal ribosome entry site (IRES) or a peptide 2A (P2A) sequence.
   Aspect 9. The cell of Aspect 8, wherein transcription of the AAV Rep coding sequences is driven by the P5 and P19 native AAV promoters, and transcription of the sequence encoding the one or more AAV capsid proteins is driven by the P40 native AAV promoter.
   Aspect 10. The cell of any one of Aspects 1-9, wherein transcription of the AAV Rep coding sequences is driven by an inducible promoter and transcription of the sequence encoding the one or more AAV capsid proteins is driven by an inducible promoter.
   Aspect 11. The cell of any one of Aspects 1-10, wherein the AAV capsid proteins comprise VP1, VP2, and VP3.
   Aspect 12. The cell of any one of Aspects 1-11, wherein the cell is a mammalian cell; optionally, wherein the mammalian cell is a HEK293 cell.
   Aspect 13. The cell of any one of Aspects 1-12, wherein the first polynucleotide construct and the third polynucleotide construct are not integrated into the nuclear genome of the cell.
   Aspect 14. The cell of any one of Aspects 1-13, wherein the inducible promoter in the second polynucleotide construct is selected from the group consisting of a tetracycline-inducible promoter, an ecdysone-inducible promoter, and a cumate-inducible promoter.
   Aspect 15. The cell of Aspect 14, wherein the first triggering agent for inducing the tetracycline-inducible promoter is tetracycline or doxycycline.
   Aspect 16. The cell of any one of Aspects 1-15, wherein the inducible recombinase is fused to an estrogen response element (ER) and translocates to the nucleus in the presence of tamoxifen.
   Aspect 17. The cell of any one of Aspects 1-16, wherein the second triggering agent for translocating the inducible recombinase is a hormone, optionally, wherein the second triggering agent is tamoxifen.
   Aspect 18. The cell of any one of Aspects 1-17, wherein the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase.
   Aspect 19. The cell of any one of Aspects 1-18, wherein upon expression of the inducible recombinase in the presence of the second triggering agent, the inducible recombinase translocates to the nucleus,
recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; and optionally,
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein.
   Aspect 20. The cell of any one of Aspects 1-19, wherein the second polynucleotide construct further comprises an insert comprising a sequence encoding VA-RNA; or optionally, wherein a fifth construct comprises an insert comprising a sequence encoding VA-RNA.
   Aspect 21. The cell of Aspect 20, wherein the VA-RNA is wild-type VA-RNA or VA-RNA comprising one or more mutations in the VA-RNA internal promoter.
   Aspect 22. The cell of Aspect 20 or 21, wherein the insert comprises:
a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and
a VA-RNA coding sequence, wherein excision of the second excisable element by the inducible recombinase generates a functional complete second constitutive promoter operably linked to the VA-RNA coding sequence to allow expression of the VA-RNA.
   Aspect 23. The cell of Aspect 22, wherein
the first part of the second constitutive promoter comprises a distal sequence element (DSE) of an RNA polymerase III promoter, and the second part of the second constitutive promoter comprises a proximal sequence element (PSE) of an RNA polymerase III promoter;
the first part of the second constitutive promoter comprises a distal sequence element (DSE) of a U6 promoter, and the second part of the second constitutive promoter comprises a proximal sequence element (PSE) of a U6 promoter; or
the first part of the second constitutive promoter comprises a distal sequence element (DSE) of a U7 promoter, and the second part of the second constitutive promoter comprises a proximal sequence element (PSE) of a U7 promoter.
   Aspect 24. The cell of any one of Aspects 1-23, wherein the first polynucleotide construct further comprises:
(i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or
(ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site.
   Aspect 25. The cell of Aspect 24, wherein first part of the AAV Rep coding sequence comprises a p5 internal promoter and a p19 internal promoter, and the second part of the AAV Rep coding sequence comprises a p40 internal promoter.
   Aspect 26. The cell of Aspect 25, wherein the excisable spacer is inserted at an insertion site between the p19 internal promoter and the p40 internal promoter of the AAV Rep coding sequence.
   Aspect 27. The cell of Aspect 26, wherein the insertion site is between a CAG and a G, a CAG and an A, an AAG and a G, and an AAG and an A.
   Aspect 28. The cell of any one of Aspects 1-27, wherein the first plasmid comprises at least 5%, 10%, 20%, 30%, 40%, 50%, 60%, 70%, 75%, 80%, 85%, 90%, 95%, or 100% sequence identity to SEQ ID NO: 1; SEQ ID NO: 2; SEQ ID NO: 3; SEQ ID NO: 4.
   Aspect 29. The cell of any one of Aspects 1-28, wherein the first polynucleotide construct comprises at least 5%, 10%, 20%, 30%, 40%, 50%, 60%, 70%, 75%, 80%, 85%, 90%, 95%, or 100% sequence identity to SEQ ID NO: 1; SEQ ID NO: 2; SEQ ID NO: 3; SEQ ID NO: 4.
   Aspect 30. The cell of any one of Aspects 1-29, wherein the second polynucleotide construct comprises at least 5%, 10%, 20%, 30%, 40%, 50%, 60%, 70%, 75%, 80%, 85%, 90%, 95%, or 100% sequence identity to SEQ ID NO: 5; SEQ ID NO: 6; SEQ ID NO: 7; SEQ ID NO: 8; or SEQ ID NO: 9-SEQ ID NO: 13.
   Aspect 31. The cell of any one of Aspects 1-30, wherein the third polynucleotide construct comprises at least 5%, 10%, 20%, 30%, 40%, 50%, 60%, 70%, 75%, 80%, 85%, 90%, 95%, or 100% sequence identity to SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 30; SEQ ID NO: 31; SEQ ID NO: 33; SEQ ID NO: 34; SEQ ID NO: 35; SEQ ID NO: 36; SEQ ID NO: 38; SEQ ID NO: 39; SEQ ID NO: 108; optionally, wherein the sequence encoding progranulin is replace with a sequence encoding dystrophin; further optionally, wherein the sequence encoding dystrophin encodes for a short, functional dystrophin.
   Aspect 33. The cell of any one of Aspects 1-32, wherein the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 30; SEQ ID NO: 31; SEQ ID NO: 33; SEQ ID NO: 34; SEQ ID NO: 35; SEQ ID NO: 36; SEQ ID NO: 38; SEQ ID NO: 39; SEQ ID NO: 108; optionally, wherein the sequence encoding progranulin is replace with a sequence encoding dystrophin; further optionally, wherein the sequence encoding dystrophin encodes for a short, functional dystrophin.
   Aspect 34. The cell any one of Aspect 1-33, wherein the third polynucleotide construct further comprises a spacer between the 5′ ITR and the sequence encoding the third selectable marker or a spacer between the sequence encoding the third selectable marker and the 3′ ITR, or a combination thereof.
   Aspect 35. The cell of Aspect 34, wherein the spacer ranges in length from 500 base pairs to 5000 base pairs.
   Aspect 36. A system for inducibly producing recombinant adenovirus associated virus (rAAV) virions, the system comprising:
(a) a first plasmid according to any one of Aspects 1-35;
(b) a cell comprising the second polynucleotide construct integrated into the nuclear genome of the cell according to any one of Aspects 1-35; and
(c) a second plasmid according to any one of Aspects 1-35;
optionally further comprising (d) a fourth polynucleotide construct according to any one of Aspects 5-35; and further optionally comprising a fifth polynucleotide construct according to any one of Aspects 20-35.
   Aspect 37. A system for inducibly producing recombinant adenovirus associated virus (rAAV) virions, the system comprising:
(a) the first polynucleotide construct in the first plasmid according to any one of Aspects 1-35;
(b) a cell comprising the second polynucleotide construct integrated into the nuclear genome of the cell according to any one of Aspects 1-35; and
(c) the third polynucleotide construct in the second plasmid according to any one of Aspects 1-35;
optionally further comprising (d) a fourth polynucleotide construct according to any one of Aspects 5-35; and further optionally comprising a fifth polynucleotide construct according to any one of Aspects 20-35.
   Aspect 38. The system of Aspects 36 or 37, further comprising the first triggering agent; optionally, wherein the first triggering agent is doxycycline.
   Aspect 39. The system of any one of Aspects 36-38, further comprising the second triggering agent; optionally, wherein the second triggering agent is tamoxifen.
   Aspect 40. A method of generating a cell for inducibly producing recombinant AAV (rAAV) virions comprising a payload, the method comprising:
introducing into a cell a second polynucleotide construct according to any one of Aspects 1-35; selecting for cells expressing the second selectable marker;
introducing into a cell of the cells expressing the second selectable marker the first polynucleotide construct according to any one of Aspects 1-35 and the third polynucleotide construct according to any one of Aspects 1-35, optionally, wherein the introducing is via transient transfection;
thereby generating the cell inducibly producing recombinant AAV (rAAV) virions comprising a payload wherein the first polynucleotide construct is integrated into the nuclear genome of the cell and the first polynucleotide construct and the third polynucleotide construct are not integrated into the genome of the cell.
   Aspect 41. A method of generating a cell for inducibly producing recombinant AAV (rAAV) virions comprising a payload, the method comprising:
introducing into a cell a second polynucleotide construct according to any one of Aspects 1-35; selecting for cells expressing the second selectable marker;
introducing into a cell of the cells expressing the second selectable marker the first plasmid according to any one of Aspects 1-35 and the second plasmid according to any one of Aspects 1-35, optionally, wherein the introducing is via transient transfection;
thereby generating the cell inducibly producing recombinant AAV (rAAV) virions comprising a payload wherein the first polynucleotide construct is integrated into the nuclear genome of the cell and the first plasmid and the plasmid are not integrated into the genome of the cell.
   Aspect 42. The method of Aspect 40 or 41, further comprising contacting the cell for inducibly producing recombinant AAV (rAAV) virions comprising a payload with the first triggering agent and the second triggering agent, wherein in the presence of the first triggering agent, the activator activates the inducible promoter resulting in expression of the inducible recombinase, wherein in the presence of the second triggering agent, the inducible recombinase translocates to the nucleus, wherein recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; thereby inducibly producing recombinant AAV (rAAV) virions comprising a payload; optionally, further comprising
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein.
   Aspect 43. The method of any one of Aspects 40-42, wherein the cell is a mammalian cell.
   Aspect 44. The method of Aspect 43, wherein the mammalian cell is a HEK293 cell.
   Aspect 45. The method of any one of Aspects 40-44, wherein the first polynucleotide construct is in a plasmid comprising at least 70%, 75%, 80%, 85%, 90%, 95%, or 100% sequence identity to SEQ ID NO: 3.
   Aspect 46. The method of any one of Aspects 40-45, wherein the second polynucleotide construct is in a plasmid comprising at least 70%, 75%, 80%, 85%, 90%, 95%, or 100% sequence identity to SEQ ID NO: 12.
   Aspect 47. The method of any one of Aspects 40-46, wherein the third polynucleotide construct is in a plasmid comprising at least 70%, 75%, 80%, 85%, 90%, 95%, or 100% sequence identity to SEQ ID NO: 15; optionally, wherein the sequence encoding progranulin is replaced with a sequence encoding dystrophin; further optionally, wherein the sequence encoding dystrophin encodes for a short, functional dystrophin.
   Aspect 48. The method of any one of Aspects 40-46, wherein the third polynucleotide construct is in a plasmid comprising at least 70%, 75%, 80%, 85%, 90%, 95%, or 100% sequence identity to SEQ ID NO: 17; optionally, wherein the sequence encoding progranulin is replaced with a sequence encoding dystrophin; further optionally, wherein the sequence encoding dystrophin encodes for a short, functional dystrophin.
   Aspect 49. The method of any one of Aspects 40-46, wherein the third polynucleotide construct is in a plasmid comprising at least 70%, 75%, 80%, 85%, 90%, 95%, or 100% sequence identity to SEQ ID NO: 19; optionally, wherein the sequence encoding progranulin is replaced with a sequence encoding dystrophin; further optionally, wherein the sequence encoding dystrophin encodes for a short, functional dystrophin.
   Aspect 50. The method of any one of Aspects 40-46, wherein the third polynucleotide construct is in a plasmid comprising at least 70%, 75%, 80%, 85%, 90%, 95%, or 100% sequence identity to SEQ ID NO: 21.
   Aspect 51. A method for generating a recombinant adenovirus associated virus (rAAV) virion comprising a sequence encoding a payload, the method comprising contacting the cell according to any one of Aspects 1-35 with the first triggering agent and the second triggering agent, wherein in the presence of the first triggering agent, the activator activates the inducible promoter of the second polynucleotide construct resulting in expression of the inducible recombinase, wherein in the presence of the second triggering agent, the inducible recombinase translocates to the nucleus, wherein recombination between the first recombination site and the second recombination site in the first polynucleotide construct by the inducible recombinase results in excision of the excisable element or inversion of the inversible element, wherein the inducible promoter becomes operably linked to the sequence encoding the one or more AAV helper proteins to allow expression of the one or more AAV helper proteins; and optionally, the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein, and recombination between the third recombination site and the fourth recombination site in the second polynucleotide construct by the inducible recombinase results in excision of the self-excising element comprising the sequence encoding the inducible recombinase, wherein the expression of the one or more AAV helper proteins results in expression of the one or more Rep proteins and the one or more capsid proteins, thereby generating an rAAV virion comprising the sequence encoding the payload of interest.
   Aspect 52. The method of Aspect 51, wherein the payload is progranulin or dystrophin; optionally, wherein the dystrophin is a short, functional dystrophin.
   Aspect 53. The method of Aspects 51 or 52, wherein the first triggering agent is doxycycline.
   Aspect 54. The method of any one of Aspects 51-52, wherein the second triggering agent is tamoxifen.

### EXAMPLES

The following examples are included for illustrative purposes only and are not intended to limit the scope of the invention.

### Example 1

Stable AAV Helper Cell Line Using Dual Transient Transfection for Production of rAAV Encapsidating a Sequence Coding for Dystrophin

This example describes the production of rAAV encapsidating a sequence coding for dystrophin from a dual transiently transfected stable AAV helper cell line after induction (see, e.g., FIG. 2). The encoded dystrophin is a shortened, functional form of dystrophin.

A stable AAV helper cell line is produced. Briefly, a suspension culture of HEK 293 cells are transfected with a plasmid comprising inducible adenoviral helper genes, such as a plasmid comprising a construct of SEQ ID NO: 12. These cells are then cultured in puromycin to select for cells that stably integrated the construct of SEQ ID NO: 12 to produce the stable AAV helper cell line.

This stable AAV helper cell line then undergoes dual transient transfection and induction, in which the stable AAV helper cell line is transfected with two plasmids via transient transfection methods: a plasmid encoding AAV Rep and Cap proteins and a plasmid encoding a payload flanked by ITRs (dystrophin plasmid), and then is induced with doxycycline and tamoxifen to produce rAAV encapsidating a sequence coding for dystrophin. The titer of the produced rAAV encapsidating a sequence coding for dystrophin is higher than the titer produced by triple transient transfection methods. The homogeneity of titer of the produced rAAV encapsidating a sequence coding for dystrophin is higher than the homogeneity of titer produced by triple transient transfection methods. The infectivity of the produced rAAV encapsidating a sequence coding for dystrophin is higher than the infectivity of virions produced by triple transient transfection methods.

Cells are infected by the produced rAAV encapsidating a sequence coding for dystrophin and subsequently, dystrophin is produced by the infected cells at a therapeutically relevant level.

### Example 2

Stable AAV Helper Cell Line Using Dual Transient Transfection for Production of rAAV Encapsidating a Sequence Coding for Progranulin

This example describes the production of rAAV encapsidating a sequence coding for progranulin from a dual transiently transfected stable AAV helper cell line after induction (see, e.g., FIG. 2).

A stable AAV helper cell line is produced. Briefly, a suspension culture of HEK 293 cells are transfected with a plasmid comprising inducible adenoviral helper genes, such as a plasmid comprising a construct of SEQ ID NO: 12. These cells are then cultured in puromycin to select for cells that stably integrated the construct of SEQ ID NO: 12 to produce the stable AAV helper cell line.

This stable AAV helper cell line then undergoes dual transient transfection and induction, in which the stable AAV helper cell line is transfected with two plasmids via transient transfection methods: a plasmid encoding AAV Rep and Cap proteins and a plasmid encoding a payload flanked by ITRs (progranulin plasmid, e.g., SEQ ID NO: 15; SEQ ID NO: 17; SEQ ID NO: 19; or SEQ ID NO: 21), and then is induced with doxycycline and tamoxifen to produce rAAV encapsidating a sequence coding for progranulin. The titer of the produced rAAV encapsidating a sequence coding for progranulin is higher than the titer produced by triple transient transfection methods. The homogeneity of titer of the produced rAAV encapsidating a sequence coding for progranulin is higher than the homogeneity of titer produced by triple transient transfection methods. The infectivity of the produced rAAV encapsidating a sequence coding for progranulin is higher than the infectivity of virions produced by triple transient transfection methods.

Cells are infected by the produced rAAV encapsidating a sequence coding for progranulin and subsequently, progranulin is produced by the infected cells at a therapeutically relevant level.

### Example 3

Stable AAV Helper Cell Line with Dual Transient Transfection for AAV Rep and Cap Proteins and Payload

A cell is transfected with an integrating vector encoding AAV Helper proteins. The polynucleotide integrated into the nuclear genome includes a second constitutive promoter operably linked to a sequence encoding an activator, wherein the cell constitutively expresses the activator and the activator is unable to activate an inducible promoter in absence of a first triggering agent, a third constitutive promoter operably linked to a sequence encoding a second selectable marker, wherein the cell constitutively expresses the second selectable marker, and from 5′ to 3′:
(i) the inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; and
(ii) a sequence encoding one or more AAV helper proteins, wherein the inducible promoter is not operably linked to the sequence encoding the one or more AAV helper proteins.
   Integration of this polynucleotide into the nuclear genome of the cell allows for propagation and/or selection of cells with this polynucleotide on a selection media (e.g., media containing an antibiotic).

Cells are further transfected with one or more vectors, plasmids, or other constructs that do not integrate into the nuclear genome of the cell. A first plasmid comprises a first polynucleotide construct comprising an AAV Rep coding sequence, a sequence encoding one or more AAV capsid proteins, and a first constitutive promoter operably linked to a sequence encoding a first selectable marker, optionally, wherein the first polynucleotide construct comprises:
(i) from 5′ to 3′:
one or more promoters operably linked to a first sequence comprising a first part of the AAV Rep coding sequence;
a second sequence comprising a second part of the AAV Rep coding sequence, wherein the one or more promoters are not operably linked to the second sequence comprising the second part of the AAV Rep coding sequence,
wherein the first sequence and the second sequence are separated by (A) an excisable element that comprises a first recombination site and a second recombination site flanking a coding sequence encoding a stop signaling sequence, wherein the first recombination site and the second recombination site are oriented in the same direction or (B) an inversible element that comprises a first recombination site and a second recombination site flanking a coding sequence encoding a stop signaling sequence,
wherein the first recombination site and the second recombination site are oriented in opposite directions;
a third sequence comprising the sequence encoding one or more AAV capsid proteins,
wherein the second sequence comprises a promoter that is operably linked to the third sequence; and
the first constitutive promoter operably linked to a sequence encoding the first selectable marker; or
(ii) from 5′ to 3′:
one or more promoters operably linked to a sequence comprising a first part of an AAV Rep coding sequence, a 5′ splice site, a first part of an intron, a first recombination site, a first 3′ splice site, a coding sequence comprising a stop signaling sequence, a second recombination site, a second part of the intron, a second 3′ splice site, and a sequence comprising a second part of the AAV Rep coding sequence,
wherein the first recombination site, the first 3′ splice site, the coding sequence comprising the stop signaling sequence, and the second recombination site form an excisable element, wherein the first recombination site and the second recombination site are oriented in the same direction, and wherein the one or more promoters are not operably linked to the sequence comprising the second part of the AAV Rep coding sequence,
wherein the first and second recombination sites are recombined by the inducible recombinase in the presence of a first triggering agent and a second triggering agent resulting in excision of the excisable element, and the first part of the AAV Rep coding sequence and the first part of the intron are joined to the second part of the intron and the second part of the AAV Rep coding sequence to form a complete AAV Rep coding sequence, allowing expression of AAV Rep proteins;
a third sequence comprising the sequence encoding one or more AAV capsid proteins,
wherein the second sequence comprises a promoter that is operably linked to the third sequence; and
the first constitutive promoter operably linked to a sequence encoding the first selectable marker.

A second plasmid comprises a third polynucleotide construct comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR).

In these cells, absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence of the first triggering agent and the second triggering agent, the cell expresses a fusion protein comprising a Rep protein encoded by the first part of the AAV Rep coding sequence that terminates at the stop signaling sequence.

### Example 4

Stable AAV Helper and AAV Rep/Cap Cell Line with Single Transient Transfection for a Payload

A cell is transfected with an integrating vector encoding AAV Helper proteins (as described in Example 3) and an integrating vector encoding AAV Rep and/or Cap proteins. In such embodiments, the vector encoding AAV Rep and/or Cap proteins comprises a first polynucleotide construct comprising an AAV Rep coding sequence a polynucleotide (as described in Example 3).

Integration of one or both of these polynucleotides into the nuclear genome of the cell allows for propagation and/or selection of cells with this polynucleotide on a selection media (e.g., media containing an antibiotic).

The cell is further transfected with a non-integrating plasmid comprising a third polynucleotide construct comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR).

In these cells, in absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence.

### Example 5

Stable AAV Helper and Payload Cell Line with Single Transient Transfection of AAV Rep/Cap

A cell is transfected with an integrating vector encoding AAV Helper proteins (as described in Example 3) and an integrating vector encoding a payload, where the payload vector comprises a third polynucleotide construct integrated into the nuclear genome of the cell, comprising a promoter operably linked to a sequence encoding a payload and a fourth constitutive promoter operably linked a third selectable marker, wherein the sequence encoding the payload is flanked by a 5′ AAV inverted terminal repeat (5′ ITR) and a 3′ AAV inverted terminal repeat (3′ ITR). The cell is further transfected with a non-integrating plasmid encoding AAV Rep and/or Cap proteins. The non-integrating plasmid encoding AAV Rep and/or Cap proteins is either a commercially available plasmid or a first plasmid as described in Example 3.

In these cells, absence of activation of the inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent and the second triggering agent, the cell expresses a fusion protein comprising a Rep protein encoded by the first part of the AAV Rep coding sequence that terminates at the stop signaling sequence.

### Example 6

rAAV Production Using a Stable Cell

In any one of the cells and/or cell lines described in Examples 3-5, addition of one or more triggering agents induces expression of one or more genes that allow for rAAV particle production and encapsidation of a sequence encoding a payload. In providing the triggering agents, expression of the inducible recombinase occurs, and the inducible recombinase translocates to the nucleus and recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein and an AAV Cap protein.

### Example 7

Stable AAV Helper Cell Line with Dual Transient Transfection for AAV Rep and Cap Proteins and Payload

A cell is transfected with an integrating vector encoding AAV Helper proteins. The polynucleotide integrated into the nuclear genome includes a second constitutive promoter operably linked to a sequence encoding an activator, wherein the cell constitutively expresses the activator and the activator is unable to activate an inducible promoter in absence of a first triggering agent, a third constitutive promoter operably linked to a sequence encoding a second selectable marker, wherein the cell constitutively expresses the second selectable marker, and from 5′ to 3′:
(i) the inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; and
(ii) a sequence encoding one or more AAV helper proteins, wherein the inducible promoter is not operably linked to the sequence encoding the one or more AAV helper proteins.
   Integration of this polynucleotide into the nuclear genome of the cell allows for propagation and/or selection of cells with this polynucleotide on a selection media (e.g., media containing an antibiotic).

Cells are further transfected with one or more vectors, plasmids, or other constructs that do not integrate into the nuclear genome of the cell. A first plasmid comprises a first polynucleotide construct comprising an AAV Rep coding sequence, a sequence encoding one or more AAV capsid proteins, and a first constitutive promoter operably linked to a sequence encoding a first selectable marker, optionally, wherein the first polynucleotide construct comprises:
(i) from 5′ to 3′:
   (A) one or more promoters operably linked to a first sequence comprising a first part of the AAV Rep coding sequence;
   a second sequence comprising a second part of the AAV Rep coding sequence,
   wherein the one or more promoters are not operably linked to the second sequence comprising the second part of the AAV Rep coding sequence,
   wherein the first sequence and the second sequence are separated by (I) an excisable element that comprises a first recombination site and a second recombination site flanking a coding sequence encoding a stop signaling sequence, wherein the first recombination site and the second recombination site are oriented in the same direction or (II) an inversible element that comprises a first recombination site and a second recombination site flanking a coding sequence encoding a stop signaling sequence, wherein the first recombination site and the second recombination site are oriented in opposite directions; or
   (B) one or more promoters operably linked to a sequence comprising a first part of an AAV Rep coding sequence, a 5′ splice site, a first part of an intron, a first recombination site, a first 3′ splice site, a coding sequence comprising a stop signaling sequence, a second recombination site, a second part of the intron, a second 3′ splice site, and a sequence comprising a second part of the AAV Rep coding sequence,
   wherein the first recombination site, the first 3′ splice site, the coding sequence comprising the stop signaling sequence, and the second recombination site form an excisable element, wherein the first recombination site and the second recombination site are oriented in the same direction, and wherein the one or more promoters are not operably linked to the sequence comprising the second part of the AAV Rep coding sequence,
   wherein the first and second recombination sites are recombined by the inducible recombinase in the presence of a first triggering agent and a second triggering agent resulting in excision of the excisable element, and the first part of the AAV Rep coding sequence and the first part of the intron are joined to the second part of the intron and the second part of the AAV Rep coding sequence to form a complete AAV Rep coding sequence, allowing expression of AAV Rep proteins;
   (ii) a third sequence comprising the sequence encoding the one or more AAV capsid proteins operably linked to a first inducible promoter; optionally, further comprising a polyadenylation signal sequence, wherein the polyadenylation signal sequence encodes a stronger polyadenylation signal than a native AAV Cap polyadenylation signal sequence and is a 3′ of the sequence encoding the one or more AAV capsid proteins; and
   (iii) the first constitutive promoter operably linked to a sequence encoding the first selectable marker.

A second plasmid comprises a third polynucleotide construct comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR).

In these cells, absence of activation of the second inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent the cell does not express detectable levels of the one or more AAV capsid proteins and in the absence of the second triggering agent, the cell expresses a fusion protein comprising a Rep protein encoded by the first part of the AAV Rep coding sequence that terminates at the stop signaling sequence.

### Example 8

Stable AAV Helper and AAV Rep/Cap Cell Line with Single Transient Transfection for a Payload

A cell is transfected with an integrating vector encoding AAV Helper proteins (as described in Example 7) and an integrating vector encoding AAV Rep and/or Cap proteins. In such embodiments, the vector encoding AAV Rep and/or Cap proteins comprises a first polynucleotide construct comprising an AAV Rep coding sequence a polynucleotide (as described in Example 7).

Integration of one or both of these polynucleotides into the nuclear genome of the cell allows for propagation and/or selection of cells with this polynucleotide on a selection media (e.g., media containing an antibiotic).

The cell is further transfected with a non-integrating plasmid comprising a third polynucleotide construct comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR).

In these cells, in absence of activation of the second inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent the cell does not express detectable levels of the one or more AAV capsid proteins and in the absence of the second triggering agent, the cell expresses a fusion protein comprising a Rep protein encoded by the first part of the AAV Rep coding sequence that terminates at the stop signaling sequence.

### Example 9

Stable AAV Helper and Payload Cell Line with Single Transient Transfection of AAV Rep/Cap

A cell is transfected with an integrating vector encoding AAV Helper proteins (as described in Example 7) and an integrating vector encoding a payload, where the payload vector comprises a third polynucleotide construct integrated into the nuclear genome of the cell, comprising a promoter operably linked to a sequence encoding a payload and a fourth constitutive promoter operably linked a third selectable marker, wherein the sequence encoding the payload is flanked by a 5′ AAV inverted terminal repeat (5′ ITR) and a 3′ AAV inverted terminal repeat (3′ ITR). The cell is further transfected with a non-integrating plasmid encoding AAV Rep and/or Cap proteins. The non-integrating plasmid encoding AAV Rep and/or Cap proteins is either a commercially available plasmid or a first plasmid as described in Example 3.

In these cells, absence of activation of the second inducible promoter by the first triggering agent, the cell does not express detectable levels of the inducible recombinase and the one or more AAV helper proteins, wherein in the absence of the second triggering agent, the inducible recombinase is unable to translocate to the nucleus; and optionally wherein in absence the first triggering agent the cell does not express detectable levels of the one or more AAV capsid proteins and in the absence of the second triggering agent, the cell expresses a fusion protein comprising a Rep protein encoded by the first part of the AAV Rep coding sequence that terminates at the stop signaling sequence.

### Example 10

rAAV Production Using a Stable Cell

In any one of the cells and/or cell lines described in Examples 7-9, addition of one or more triggering agents induces expression of one or more genes that allow for rAAV particle production and encapsidation of a sequence encoding a payload. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 11

System for rAAV Production with an Integrated AAV Helper Construct

This example describes a system for rAAV production. This system includes a cell comprising an polynucleotide encoding AAV Helper proteins is incorporated into its nuclear genome, such as described in Examples 3-6. The system further includes a polynucleotide encoding AAV Rep and/or Cap proteins and a polynucleotide encoding a payload, such as described in Example 3. The polynucleotides encoding AAV Rep and/or Cap proteins and a payload can be a plasmid or other construct.

The polynucleotides and/or plasmids are transfected into the cell, which allows the cell to be capable of producing rAAV particles. The cells are induced using the relevant triggering agent(s), such as doxycycline and tamoxifen. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 12

System for rAAV Production with an Integrated AAV Helper Construct and an Integrated AAV Rep and/or Cap Construct

This example includes a cell where a polynucleotide encoding AAV Rep and/or Cap protein and a polynucleotide encoding AAV Helper proteins are integrated into the nuclear genome of the cell, such as described in Example 4. This system further includes a polynucleotide comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR)—this polynucleotide can be a plasmid or other construct.

The polynucleotide and/or plasmid is transfected into the cell, which allows the cell to be capable of producing rAAV particles. The cells are induced using the relevant triggering agent(s), such as doxycycline and tamoxifen. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 13

System for rAAV Production with an Integrated AAV Helper Construct and an Integrated Payload Construct

This example describes a system comprising a cell where a polynucleotide encoding AAV Helper proteins and a polynucleotide encoding are integrated into the nuclear genome of the cell, such as described in Example 5. This system further includes a polynucleotide encoding AAV Rep and/or Cap proteins. The polynucleotide encoding AAV Rep and/or Cap proteins can be a commercially available construct and/or a polynucleotide as described in Example 3. The polynucleotide encoding AAV Rep and/or Cap proteins can be a plasmid or other construct.

The polynucleotide and/or plasmid is transfected into the cell, which allows the cell to be capable of producing rAAV particles. The cells are induced using the relevant triggering agent(s), such as doxycycline and tamoxifen. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 14

System for rAAV Production with an Integrated AAV Helper Construct

This example describes a system for rAAV production. This system includes a cell comprising an polynucleotide encoding AAV Helper proteins is incorporated into its nuclear genome, such as described in Examples 7-10. The system further includes a polynucleotide encoding AAV Rep and/or Cap proteins and a polynucleotide encoding a payload, such as described in Example 7. The polynucleotides encoding AAV Rep and/or Cap proteins and a payload can be a plasmid or other construct.

The polynucleotides and/or plasmids are transfected into the cell, which allows the cell to be capable of producing rAAV particles. The cells are induced using the relevant triggering agent(s), such as doxycycline and tamoxifen. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 15

System for rAAV Production with an Integrated AAV Helper Construct and an Integrated AAV Rep and/or Cap Construct

This example describes a system for rAAV production. This system includes a cell comprising an polynucleotide encoding AAV Helper proteins is incorporated into its nuclear genome, such as described in Examples 7-10. The system further includes a polynucleotide encoding AAV Rep and/or Cap proteins and a polynucleotide encoding a payload, such as described in Example 7. The polynucleotides encoding AAV Rep and/or Cap proteins and a payload can be a plasmid or other construct.

The polynucleotide and/or plasmid are transfected into the cell, which allows the cell to be capable of producing rAAV particles. The cells are induced using the relevant triggering agent(s), such as doxycycline and tamoxifen. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 16

System for rAAV Production with an Integrated AAV Helper Construct and an Integrated Payload Construct

This example describes a system comprising a cell where a polynucleotide encoding AAV Helper proteins and a polynucleotide encoding are integrated into the nuclear genome of the cell, such as described in Example 9. This system further includes a polynucleotide encoding AAV Rep and/or Cap proteins. The polynucleotide encoding AAV Rep and/or Cap proteins is a polynucleotide as described in Example 7. The polynucleotide encoding AAV Rep and/or Cap proteins can be a plasmid or other construct.

The polynucleotide and/or plasmid are transfected into the cell, which allows the cell to be capable of producing rAAV particles. The cells are induced using the relevant triggering agent(s), such as doxycycline and tamoxifen. In providing the triggering agents, expression of the inducible recombinase occurs, and the inducible recombinase translocates to the nucleus, and recombination between the third recombination site and the fourth recombination site in the second polynucleotide construct results in excision of the self-excising element comprising the sequence encoding the inducible recombinase, wherein the second inducible promoter becomes operably linked to the sequence encoding the one or more AAV helper proteins to allow expression of the one or more AAV helper proteins; and optionally, (i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or (ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters of the first construct are operably linked to the complete AAV Rep coding sequence to allow expression of an AAV Rep protein; and in the presence of the first triggering agent, the activator activates the first inducible promoter resulting in expression of the one or more AAV capsid proteins.

### Example 17

Generating a Cell for rAAV production Having an Integrated Construct

Generating a cell for inducibly producing recombinant AAV (rAAV) virions comprising a payload includes introducing one or two polynucleotide constructs for genomic integration, such as polynucleotide encoding AAV Rep and/or Cap proteins from Example 4 or Example 8, a polynucleotide encoding AAV Helper proteins from Examples 3-5 and Examples 7-9, and/or a polynucleotide encoding a payload from Examples 3-5 and Examples 7-9. Once a first polynucleotide is transfected and integrated, the cell can be selected for expression of one or more selectable markers, depending on how many constructs have been integrated. The integration of more than one polynucleotide can occur in series, rather than parallel, such that the cell undergoes multiple rounds of transfection/integration and selection.

One or two polynucleotide constructs, such as a plasmid encoding an AAV Rep and/or Cap proteins and/or a plasmid encoding a payload (such as in Example 3 or Example 7) is transiently transfected into the cell. The cell is then selected for the presence of the transient polynucleotide using a selectable marker.

The cell with one or more integrated polynucleotides and one or two transiently transfected polynucleotides are then induced to produce rAAV particles.

### Example 18

Cells comprising One or More Integrated Constructs and Using an Exogenous Recombinase

This example describes using an exogenously applied recombinase (such as cre recombinase) to excise the excisable element from one or more constructs described in Examples 3-5 and Examples 7-9. Without the recombinase, the cells produce a fusion Rep protein that terminates at a stop codon. In the presence of the recombinase, the full length Rep proteins are produced.

### Example 19

Systems comprising a Cell with One or More Integrated Constructs and Using an Exogenous Recombinase

This example describes a system for rAAV production comprising a cell with one or more integrated constructs, such as describe in Examples 3-5 and Examples 7-9 and or the systems of Examples 11-16. This example includes a recombinase, such as cre recombinase, and one or more triggering agents, such as doxycycline and tamoxifen.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1-214. (canceled)

215. A method for generating a recombinant adenovirus associated virus (rAAV) virion comprising a sequence encoding a payload, the method comprising:
contacting a cell to a first triggering agent and a second triggering agent, wherein the cell comprises:
(a) a first polynucleotide construct comprising an AAV Rep coding sequence, a sequence encoding one or more AAV Cap proteins, and a first constitutive promoter operably linked to a sequence encoding a first selectable marker, optionally, wherein the first polynucleotide construct comprises:
(i) from 5′ to 3′:
(A) one or more promoters operably linked to a first sequence comprising a first part of the AAV Rep coding sequence;
a second sequence comprising a second part of the AAV Rep coding sequence, wherein the one or more promoters are not operably linked to the second sequence comprising the second part of the AAV Rep coding sequence,
wherein the first sequence and the second sequence are separated by (I) an excisable element that comprises a first recombination site and a second recombination site flanking a coding sequence encoding a stop signaling sequence, wherein the first recombination site and the second recombination site are oriented in the same direction or (II) an inversible element that comprises a first recombination site and a second recombination site flanking a coding sequence encoding a stop signaling sequence, wherein the first recombination site and the second recombination site are oriented in opposite directions; or
(B) one or more promoters operably linked to a sequence comprising a first part of an AAV Rep coding sequence, a 5′ splice site, a first part of an intron, a first recombination site, a first 3′ splice site, a coding sequence comprising a stop signaling sequence, a second recombination site, a second part of the intron, a second 3′ splice site, and a sequence comprising a second part of the AAV Rep coding sequence,
wherein the first recombination site, the first 3′ splice site, the coding sequence comprising the stop signaling sequence, and the second recombination site form an excisable element, wherein the first recombination site and the second recombination site are oriented in the same direction, and wherein the one or more promoters are not operably linked to the sequence comprising the second part of the AAV Rep coding sequence,
wherein the first and second recombination sites are recombined by the inducible recombinase in the presence of a first triggering agent and a second triggering agent resulting in excision of the excisable element, and the first part of the AAV Rep coding sequence and the first part of the intron are joined to the second part of the intron and the second part of the AAV Rep coding sequence to form a complete AAV Rep coding sequence, allowing expression of AAV Rep proteins;
(ii) a third sequence comprising the sequence encoding the one or more AAV Cap proteins, wherein (A) the second sequence comprises a promoter that is operably linked to the third sequence, or wherein (B) the third sequences is operably linked to a first inducible promoter;
(iii) the first constitutive promoter operably linked to a sequence encoding the first selectable marker; and
(b) a second polynucleotide construct integrated into the nuclear genome of the cell, comprising:
a second constitutive promoter operably linked to a sequence encoding an activator, wherein the cell constitutively expresses the activator and the activator is unable to activate the first inducible promoter or a second inducible promoter in absence of a first triggering agent;
from 5′ to 3′:
(i) the second inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; and
(ii) a sequence encoding one or more AAV helper proteins, wherein the inducible promoter is not operably linked to the sequence encoding the one or more AAV helper proteins; and
a third constitutive promoter operably linked to a sequence encoding a second selectable marker, wherein the cell constitutively expresses the second selectable marker; and
(c) a third polynucleotide construct comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR);
wherein in the presence of the first triggering agent, the activator activates the second inducible promoter in the second polynucleotide construct resulting in expression of the inducible recombinase, wherein in the presence of the second triggering agent, the inducible recombinase translocates to the nucleus, wherein recombination between the third recombination site and the fourth recombination site in the second polynucleotide construct by the inducible recombinase results in excision of the self-excising element comprising the sequence encoding the inducible recombinase, and wherein the second inducible promoter becomes operably linked to the sequence encoding the one or more AAV helper proteins to allow expression of the one or more AAV helper proteins; and
wherein recombination between the first recombination site and the second recombination site in the first polynucleotide construct by the inducible recombinase results in excision of the excisable element or inversion of the inversible element wherein the first part of the AAV Rep coding sequence and the second part of the AAV Rep coding sequence are joined to form a complete AAV Rep coding sequence, wherein the one or more promoters are operably linked to the complete AAV Rep coding sequence to allow expression of the one or more Rep proteins; and wherein (A) the promoter comprised within the second sequence allows expression of the one or more Cap proteins or (B) wherein in the presence of the first triggering agent, the activator activates the first inducible promoter resulting in expression of the one or more AAV Cap proteins; and
wherein inducing expression of the one or more AAV helper proteins results in expression of the one or more Rep proteins and the one or more Cap proteins, thereby generating an rAAV virion comprising the sequence encoding the payload of interest.

216. The method of claim 215, wherein either the first polynucleotide construct or the third polynucleotide construct are integrated into the nuclear genome of the cell.

217. The method of claim 215, wherein one or both of the first polynucleotide construct and the third polynucleotide construct are comprised in a plasmid and not integrated into the nuclear genome of the cell.

218. The method of claim 215, further comprising:
introducing into the cell the first polynucleotide construct, the second polynucleotide construct, and the third polynucleotide construct, wherein the second polynucleotide construct integrates into the nuclear genome of the cell, and wherein one or both of the first polynucleotide construct and the third polynucleotide construct are comprised in a plasmid and not integrated into the nuclear genome of the cell; and
selecting for a cell expressing the first selectable marker, the second selectable marker, and the third selectable marker.

219. The method of claim 215, further comprising:
introducing into the cell the second polynucleotide construct, wherein the second polynucleotide construct integrates into the nuclear genome of the cell, and
selecting for a cell expressing the second selectable marker.

220. The method of claim 219, further comprising:
introducing into the cell the first polynucleotide construct and the third polynucleotide construct, and wherein one or both of the first polynucleotide construct and the third polynucleotide construct are comprised in a plasmid and not integrated into the nuclear genome of the cell; and
selecting for a cell expressing the first selectable marker and the third selectable marker.

221. The method of claim 217, wherein the plasmid comprising the first polynucleotide comprises at least 5%, 10%, 20%, 30%, 40%, 50%, 60%, 70%, 75%, 80%, 85%, 90%, 95%, or 100% sequence identity to SEQ ID NO: 1; SEQ ID NO: 2; SEQ ID NO: 3; SEQ ID NO: 4; SEQ ID NO: 22; SEQ ID NO: 24; SEQ ID NO: 99; SEQ ID NO: 101; SEQ ID NO: 104; SEQ ID NO: 114; SEQ ID NO: 115; or SEQ ID NO: 114 and SEQ ID NO: 115.

222. The method of claim 215, wherein transcription of full-length AAV Rep coding sequences occurs after excision of the excisable element or inversion of the inversible element; optionally, wherein transcription of the sequence encoding the one or more AAV capsid proteins occurs after excision of the excisable element or inversion of the inversible element.

223. The method of claim 215, wherein transcription of the AAV Rep coding sequences is driven by the P5 and P19 native AAV promoters, and transcription of the sequence encoding the one or more AAV capsid proteins is driven by the P40 native AAV promoter comprised in the second sequence.

224. The method of claim 215, wherein the second inducible promoter is selected from the group consisting of a tetracycline-inducible promoter, an ecdysone-inducible promoter, and a cumate-inducible promoter.

225. The method of claim 215, wherein third sequence comprising the sequence encoding the one or more AAV Cap proteins is operably linked to the first inducible promoter, and the first inducible promoter is selected from the group consisting of a tetracycline-inducible promoter, an ecdysone-inducible promoter, and a cumate-inducible promoter.

226. The method of claim 215, wherein the third sequences further comprises a polyadenylation signal sequence, wherein the polyadenylation signal sequence encodes a stronger polyadenylation signal than a native AAV Cap polyadenylation signal sequence and is a 3′ of the sequence encoding the one or more AAV capsid proteins; optionally wherein the stronger polyadenylation signal enhances RNA processing, RNA stability, RNA translation efficiency, or any combination thereof.

227. The method of claim 226, wherein the polyadenylation signal sequence is a SV40 polyadenylation signal sequence, a bovine growth hormone polyadenylation signal sequence, or a Rabbit Beta Globin polyadenylation signal sequence.

228. The method of claim 226, wherein the native AAV Cap polyadenylation signal sequence has at least 70%, at least 75%, at least 80%, at least 85%, at least 90%, at least 95%, at least 97%, at least 98%, at least 99%, or 100% sequence identity to SEQ ID NO: 112; and wherein the polyadenylation signal sequence has at least 70%, at least 75%, at least 80%, at least 85%, at least 90%, at least 95%, at least 97%, at least 98%, at least 99%, or 100% sequence identity to SEQ ID NO: 111, has at least 70%, at least 75%, at least 80%, at least 85%, at least 90%, at least 95%, at least 97%, at least 98%, at least 99%, or 100% sequence identity to SEQ ID NO: 110, or has at least 70%, at least 75%, at least 80%, at least 85%, at least 90%, at least 95%, at least 97%, at least 98%, at least 99%, or 100% sequence identity to SEQ ID NO: 113.

229. The method of claim 215, wherein the first triggering agent is tetracycline or doxycycline and/or the second triggering agent is tamoxifen.

230. The method of claim 215, wherein the cell is a HEK293 cell.

231. A method for generating a recombinant adenovirus associated virus (rAAV) virion comprising a sequence encoding a payload, the method comprising:
contacting a cell to a recombinase, wherein the cell comprises:
(a) a first polynucleotide construct comprising an AAV Rep coding sequence, a sequence encoding one or more AAV Cap proteins, and a first constitutive promoter operably linked to a sequence encoding a first selectable marker, optionally, wherein the first polynucleotide construct comprises:
(i) from 5′ to 3′:
(A) one or more promoters operably linked to a first sequence comprising a first part of the AAV Rep coding sequence;
a second sequence comprising a second part of the AAV Rep coding sequence, wherein the one or more promoters are not operably linked to the second sequence comprising the second part of the AAV Rep coding sequence,
wherein the first sequence and the second sequence are separated by (I) an excisable element that comprises a first recombination site and a second recombination site flanking a coding sequence encoding a stop signaling sequence, wherein the first recombination site and the second recombination site are oriented in the same direction or (II) an inversible element that comprises a first recombination site and a second recombination site flanking a coding sequence encoding a stop signaling sequence, wherein the first recombination site and the second recombination site are oriented in opposite directions; or
(B) one or more promoters operably linked to a sequence comprising a first part of an AAV Rep coding sequence, a 5′ splice site, a first part of an intron, a first recombination site, a first 3′ splice site, a coding sequence comprising a stop signaling sequence, a second recombination site, a second part of the intron, a second 3′ splice site, and a sequence comprising a second part of the AAV Rep coding sequence,
wherein the first recombination site, the first 3′ splice site, the coding sequence comprising the stop signaling sequence, and the second recombination site form an excisable element, wherein the first recombination site and the second recombination site are oriented in the same direction, and wherein the one or more promoters are not operably linked to the sequence comprising the second part of the AAV Rep coding sequence,
wherein the first and second recombination sites are recombined by the inducible recombinase in the presence of a first triggering agent and a second triggering agent resulting in excision of the excisable element, and the first part of the AAV Rep coding sequence and the first part of the intron are joined to the second part of the intron and the second part of the AAV Rep coding sequence to form a complete AAV Rep coding sequence, allowing expression of AAV Rep proteins;
(ii) a third sequence comprising the sequence encoding the one or more AAV Cap proteins, wherein (A) the second sequence comprises a promoter that is operably linked to the third sequence, or wherein (B) the third sequences is operably linked to a first inducible promoter;
(iii) the first constitutive promoter operably linked to a sequence encoding the first selectable marker; and
(b) a second polynucleotide construct integrated into the nuclear genome of the cell, comprising:
a second constitutive promoter operably linked to a sequence encoding an activator, wherein the cell constitutively expresses the activator and the activator is unable to activate the first inducible promoter or a second inducible promoter in absence of a first triggering agent;
from 5′ to 3′:
(i) the second inducible promoter operably linked to a self-excising element; the self-excising element comprising a third recombination site and a fourth recombination site flanking a sequence encoding an inducible recombinase, wherein the inducible recombinase is unable to translocate to the nucleus in the absence of a second triggering agent, wherein the third recombination site and the fourth recombination site are oriented in the same direction; and
(ii) a sequence encoding one or more AAV helper proteins, wherein the inducible promoter is not operably linked to the sequence encoding the one or more AAV helper proteins; and
a third constitutive promoter operably linked to a sequence encoding a second selectable marker, wherein the cell constitutively expresses the second selectable marker; and
(c) a third polynucleotide construct comprising a promoter operably linked to a sequence encoding a payload and a fourth constitutive promoter operably linked to a third selectable marker, wherein the sequence encoding the payload is flanked by a 5′ AAV inverted terminal repeat (5′ ITR) and a 3′ AAV inverted terminal repeat (3′ ITR);
wherein in the presence of the recombinase, recombination between the first recombination site and the second recombination site in the first polynucleotide construct by the recombinase results in excision of the excisable element or inversion of the inversible element and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of the one or more Rep proteins; and wherein (A) the promoter comprised within the second sequence allows expression of the one or more Cap proteins or (B) wherein in the presence of the first triggering agent, the activator activates the first inducible promoter resulting in expression of the one or more AAV Cap proteins,
thereby generating an rAAV virion comprising the sequence encoding the payload of interest.

232. The method of claim 231, further comprising contacting the cell to a first triggering agent and a second triggering agent, wherein in the presence of the first triggering agent, the activator activates the second inducible promoter in the second polynucleotide construct resulting in expression of the inducible recombinase, wherein in the presence of the second triggering agent, the inducible recombinase translocates to the nucleus, wherein recombination between the third recombination site and the fourth recombination site in the second polynucleotide construct by the inducible recombinase results in excision of the self-excising element comprising the sequence encoding the inducible recombinase, and wherein the second inducible promoter becomes operably linked to the sequence encoding the one or more AAV helper proteins to allow expression of the one or more AAV helper proteins; and wherein inducing expression of the one or more AAV helper proteins results in expression of the one or more Rep proteins and the one or more Cap proteins, thereby generating an rAAV virion comprising the sequence encoding the payload of interest.

233. The method of claim 231, wherein either the first polynucleotide construct or the third polynucleotide construct are integrated into the nuclear genome of the cell.

234. The method of claim 231, wherein one or both of the first polynucleotide construct and the third polynucleotide construct are comprised in a plasmid and not integrated into the nuclear genome of the cell.

235. The method of claim 231, further comprising:
introducing into the cell the first polynucleotide construct, the second polynucleotide construct, and the third polynucleotide construct, wherein the second polynucleotide construct integrates into the nuclear genome of the cell, and wherein one or both of the first polynucleotide construct and the third polynucleotide construct are comprised in a plasmid and not integrated into the nuclear genome of the cell; and
selecting for a cell expressing the first selectable marker, the second selectable marker, and the third selectable marker.

236. The method of claim 231, further comprising:
introducing into the cell the second polynucleotide construct, wherein the second polynucleotide construct integrates into the nuclear genome of the cell, and
selecting for a cell expressing the second selectable marker.

237. The method of claim 236, further comprising:
introducing into the cell the first polynucleotide construct and the third polynucleotide construct, and wherein one or both of the first polynucleotide construct and the third polynucleotide construct are comprised in a plasmid and not integrated into the nuclear genome of the cell; and
selecting for a cell expressing the first selectable marker and the third selectable marker.

238. The method of claim 231, wherein the cell is a HEK293 cell.


SEQUENCE LISTING (123 sequences)

SEQ ID NO: 1 - DNA, 6367 bp, synthetic construct
  source 1..6367: mol_type = other DNA
ggcctccacggccactagtaacggccgccagtgtgctggaattcgccctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactaca

SEQ ID NO: 2 - DNA, 12385 bp, synthetic construct
  source 1..12385: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtaacggccgccagtgtgctggaattcgccctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacataagggcgaattctgcagatatccatcacactggcggccgctcgagcatgcatctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaggtacctcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 3 - DNA, 11646 bp, synthetic construct
  source 1..11646: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaattaagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggcgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 4 - DNA, 6397 bp, synthetic construct
  source 1..6397: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgt

SEQ ID NO: 5 - DNA, 14766 bp, synthetic construct
  source 1..14766: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 6 - DNA, 13743 bp, synthetic construct
  source 1..13743: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 7 - DNA, 10347 bp, synthetic construct
  source 1..10347: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcc

SEQ ID NO: 8 - DNA, 9344 bp, synthetic construct
  source 1..9344: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatg

SEQ ID NO: 9 - DNA, 14227 bp, synthetic construct
  source 1..14227: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaggttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 10 - DNA, 14058 bp, synthetic construct
  source 1..14058: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtcaaccctcccacacgtaaccagatgggagaagctctcgtattccaacagctgtaggttgaccgtctgaatccttcactatggcctttatacctggatgcagatccaacaggacttgtctacatctaccacaaggtgacaagatcccgcgattctcgttaccaatagcaacgatacaagtcagattccctgcggcggcggcagcggcggttcccagtactacgagttctgcgcaggggccacccgtaaaatgatacacatttaccccagtaaaaatccgcccatcggatgacaaggctgcactagccacggaatagtcgtcagatataggtatggaattgattgtggcggtagccctctctatcagcgtggactcctcctgagagaggggttttgccatggtggcggcttaagggttcgatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 11 - DNA, 14067 bp, synthetic construct
  source 1..14067: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtctagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagggcccattggtatggcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 12 - DNA, 13923 bp, synthetic construct
  source 1..13923: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggttcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 13 - DNA, 14372 bp, synthetic construct
  source 1..14372: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatggtggtagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 14 - DNA, 2366 bp, synthetic construct
  source 1..2366: mol_type = other DNA
cactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagaga

SEQ ID NO: 15 - DNA, 8271 bp, synthetic construct
  source 1..8271: mol_type = other DNA
ccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcactagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcac

SEQ ID NO: 16 - DNA, 2280 bp, synthetic construct
  source 1..2280: mol_type = other DNA
tctctgcgcgctcgctcgctcactgaggccgggcgaccaaaggtcgcccgacgcccgggctttgcccgggcggcctcagtgagcgagcgagcgcgcagaggatccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - DNA, 8195 bp, synthetic construct
  source 1..8195: mol_type = other DNA
agcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgagcttctgatagactaattgacatcatttgagtcaattccagacatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcactcagtttggaaggttgtcgactctcatcagatccagctctcgttcaaaaatttcgtcaattggcagcatctggccgtccacggtcataaacttatgatctttagtagcgcggatgagggatccgtcctcgagacagtattcaaaaacttcctgctctcctctgtcgtgccattgagccacgggctgtgtgtagatgttcccattattatcgactgaatagacggtacattcaatcctcttctccacgattttgccgataggaagcaatccgtactcgacagtcaggatctcggtctcgtatgaaaggcacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcctgttgagaaatgttaaatgttttcatggtggcgatatcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttgcggccgcggatccgatttaaattcgaattcgctagctctagtgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagataacgcaggaaagaaataaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaag

SEQ ID NO: 18 - DNA, 2445 bp, synthetic construct
  source 1..2445: mol_type = other DNA
tctctgcgcgctcgctcgctcactgaggccgggcgaccaaaggtcgcccgacgcccgggctttgcccgggcggcctcagtgagcgagcgagcgcgcagaggatccaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcaccgctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggtacccaggggtggcgggtgggtgggctgagcacagtgtggcaggcagccgggccccagtgcccacctgcccttcttcatctgccctaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 19 - DNA, 8359 bp, synthetic construct
  source 1..8359: mol_type = other DNA
gtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgagcttctgatagactaattgacatcatttgagtcaattccagacatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcactcagtttggaaggttgtcgactctcatcagatccagctctcgttcaaaaatttcgtcaattggcagcatctggccgtccacggtcataaacttatgatctttagtagcgcggatgagggatccgtcctcgagacagtattcaaaaacttcctgctctcctctgtcgtgccattgagccacgggctgtgtgtagatgttcccattattatcgactgaatagacggtacattcaatcctcttctccacgattttgccgataggaagcaatccgtactcgacagtcaggatctcggtctcgtatgaaaggcacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcctgttgagaaatgttaaatgttttcatggtggcgatatcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttgcggccgcggatccgatttaaattcgaattcgctagctctagtgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatccaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcaccgctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggtacccaggggtggcgggtgggtgggctgagcacagtgtggcaggcagccgggccccagtgcccacctgcccttcttcatctgccctaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagataagcaggaaagaaataaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacga

SEQ ID NO: 20 - DNA, 2477 bp, synthetic construct
  source 1..2477: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcgcctagaagtaaaggcaacatccactgaggagcagttctttgatttgcaccaccaccggatccgggacctgaaataaaagacaaaaagactaaacttacgctagccggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagaga

SEQ ID NO: 21 - DNA, 8377 bp, synthetic construct
  source 1..8377: mol_type = other DNA
gcgcctagaagtaaaggcaacatccactgaggagcagttctttgatttgcaccaccaccggatccgggacctgaaataaaagacaaaaagactaaacttacgctagccggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcactagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatctttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgcta

SEQ ID NO: 22 - DNA, 3952 bp, synthetic construct
  source 1..3952: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 23 - DNA, 2650 bp, synthetic construct
  source 1..2650: mol_type = other DNA
atggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 24 - DNA, 6397 bp, synthetic construct; identical to SEQ ID NO: 4
  source 1..6397: mol_type = other DNA

SEQ ID NO: 25 - DNA, 98 bp, Adeno-associated virus
  source 1..98: mol_type = genomic DNA
ggaggggtggagtcgtgacgtgaattacgtcatagggttagggaggtcctgtattagaggtcacgtgagtgttttgcgacattttgcgacaccatgtg

SEQ ID NO: 26 - DNA, 353 bp, synthetic construct
  source 1..353: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaac

SEQ ID NO: 27 - DNA, 1356 bp, synthetic construct
  source 1..1356: mol_type = other DNA
ataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttat

SEQ ID NO: 28 - DNA, 220 bp, synthetic construct
  source 1..220: mol_type = other DNA
aattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacag

SEQ ID NO: 29 - DNA, 13923 bp, synthetic construct; identical to SEQ ID NO: 12
  source 1..13923: mol_type = other DNA

SEQ ID NO: 30 - DNA, 1782 bp, synthetic construct
  source 1..1782: mol_type = other DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 31 - DNA, 2366 bp, synthetic construct; identical to SEQ ID NO: 14
  source 1..2366: mol_type = other DNA

SEQ ID NO: 32 - DNA, 4886 bp, synthetic construct
  source 1..4886: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcactagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 33 - DNA, 5182 bp, synthetic construct
  source 1..5182: mol_type = other DNA
ccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctgcaggagaccgagttggattgtctcctctaagaactcttgattacagttaaggaatctgccgatcagtggtgtgtcaaggtctacagtgagcgccgcttgactcttatctgatcaacggctataacgtggaattgccaggcttgcagatagcgttaggtattccaaccgaatagacaacacactgtgtcttgttaacagttctgctacgacggcaataacacgaagataataccggtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattccggttataacggtccaatctaacagtgatattcactgtggtatcctgttctaggttggttcttgataggtctcggatccaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatgaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 34 - DNA, 8271 bp, synthetic construct; identical to SEQ ID NO: 15
  source 1..8271: mol_type = other DNA

SEQ ID NO: 35 - DNA, 8190 bp, synthetic construct
  source 1..8190: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggtctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 36 - DNA, 2005 bp, synthetic construct
  source 1..2005: mol_type = other DNA
gcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaa

SEQ ID NO: 37 - DNA, 4639 bp, synthetic construct
  source 1..4639: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatcctctacgccatattatccacagtccaacggccaggcggaggctagtaacaaggttatcctcggcatcctccgcaggtaccatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttggtaccctcacggaccagtgcaacatattcccaacatcccgttgcagcctatcattaaaccttggccggtcgcggctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 38 - DNA, 8271 bp, synthetic construct; identical to SEQ ID NO: 15
  source 1..8271: mol_type = other DNA

SEQ ID NO: 39 - DNA, 8567 bp, synthetic construct
  source 1..8567: mol_type = other DNA
taaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatagcttctgatagactaattgacatcatttgagtcaattccagacatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcactcagtttggaaggttgtcgactctcatcagatccagctctcgttcaaaaatttcgtcaattggcagcatctggccgtccacggtcataaacttatgatctttagtagcgcggatgagggatccgtcctcgagacagtattcaaaaacttcctgctctcctctgtcgtgccattgagccacgggctgtgtgtagatgttcccattattatcgactgaatagacggtacattcaatcctcttctccacgattttgccgataggaagcaatccgtactcgacagtcaggatctcggtctcgtatgaaaggcacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcctgttgagaaatgttaaatgttttcatggtggcgatatcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatcctcatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggatccgagacctatcaagaaccaacctagaacaggataccacagtgaatatcactgttagattggaccgttataaccggaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcaccgctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcaccggtattatcttcgtgttattgccgtcgtagcagaactgttaacaagacacagtgtgttgtctattcggttggaatacctaacgctatctgcaagcctggcaattccacgttatagccgttgatcagataagagtcaagcggcgctcactgtagaccttgacacaccactgatcggcagattccttaactgtaatcaagagttcttagaggagacaatccaactcggtctcctgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaa

SEQ ID NO: 40 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 41 - protein, 373 aa, Homo sapiens
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 42 - protein, 279 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 43 - DNA, 534 bp, synthetic construct
  source 1..534: mol_type = other DNA
atgagagggtcagatccggcagcgttgaagcgggctaggaacactgaggcagcaaggcgctctcgagcaaggaagttgcaacggatgaaacaattggaagacaaagttgaagagctgctttcaaaaaactaccaccttgaaaatgaagtcgcgaggctgaagaaattggtcggatctgctggcagcgcagcggggagcggtgagtttatggtcagacctctcaactgtattgtcgctgtctcacagaacatgggtatcggaaagaacggtgacttgccgtggccgccactgcggaatgagttcaaatactttcagcgcatgacgaccaccagcagtgtggagggtaagcaaaatcttgtcataatgggtcgcaagacttggttttctattccagagaaaaacagaccgcttaaagataggattaacatcgtgttgagccgggaactgaaagagccaccaaggggagcacattttttggctaagtccttggatgacgccctgcgactgatagagcaaccagaacttgcttagtaa

SEQ ID NO: 44 - DNA, 453 bp, synthetic construct
  source 1..453: mol_type = other DNA
atgcgcggttccgacccagcagctttgaaacgagcacgaaacacggaagcagcccgcaggagtcgagcgagaaaacttcagcggatgaagcagcttgaagataaagtcgaggaattgcttagcaagaattatcacctcgagaatgaagtggcgcgactgaaaaaacttgtaggttctgctgggagcgcagccggaagcggcgagttctcaaaagttgacatggtgtggatcgtgggtggaagttctgtctatcaagaggcgatgaatcagcctggccacctcagactgtttgttacaaggatcatgcaggagttcgagtctgacacgttttttccagagatcgacctggggaaatataaactcctcccagagtacccaggagtgcttagtgaggtccaagaagagaagggaatcaaatataaatttgaagtttacgaaaagaaggattagtaa

SEQ ID NO: 45 - protein, 102 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 46 - protein, 36 aa, Nostoc punctiforme
MIKIATRKYLGKQNVYDIGVERDHNFALKNGFIASN

SEQ ID NO: 47 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 48 - protein, 338 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 49 - protein, 252 aa, synthetic construct
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 50 - protein, 366 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 51 - protein, 224 aa, synthetic construct
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 52 - protein, 385 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 53 - protein, 205 aa, synthetic construct
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 54 - protein, 435 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 55 - protein, 155 aa, synthetic construct
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 56 - protein, 154 aa, synthetic construct
MTTSASSHLNKGIKQVYMSLPQGEKVQAMYIWIDGTGEGLRCKTRTLDSEPKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 57 - protein, 357 aa, synthetic construct
MIKIATRKYLGKQNVYDIGVERDHNFALKNGFIASNCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 58 - protein, 218 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 59 - protein, 293 aa, synthetic construct
MIKIATRKYLGKQNVYDIGVERDHNFALKNGFIASNCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 60 - protein, 284 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 61 - protein, 227 aa, synthetic construct
MIKIATRKYLGKQNVYDIGVERDHNFALKNGFIASNCLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 62 - protein, 330 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 63 - protein, 181 aa, synthetic construct
MIKIATRKYLGKQNVYDIGVERDHNFALKNGFIASNCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 64 - protein, 353 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 65 - protein, 158 aa, synthetic construct
MIKIATRKYLGKQNVYDIGVERDHNFALKNGFIASNCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 66 - protein, 144 aa, synthetic construct
MPVAGSELPRRPLPPAAQERDAEPRPPHGELQYLGQIQHILRCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 67 - protein, 273 aa, synthetic construct
MIKIATRKYLGKQNVYDIGVERDHNFALKNGFIASNCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 68 - protein, 262 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 69 - protein, 155 aa, synthetic construct
MIKIATRKYLGKQNVYDIGVERDHNFALKNGFIASNCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 70 - protein, 266 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 71 - protein, 151 aa, synthetic construct
MIKIATRKYLGKQNVYDIGVERDHNFALKNGFIASNCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 72 - protein, 277 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 73 - protein, 140 aa, synthetic construct
MIKIATRKYLGKQNVYDIGVERDHNFALKNGFIASNCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 74 - DNA, 613 bp, synthetic construct
  source 1..613: mol_type = other DNA
gaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactga

SEQ ID NO: 75 - DNA, 223 bp, synthetic construct
  source 1..223: mol_type = other DNA
atgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaatt

SEQ ID NO: 76 - protein, 724 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 77 - protein, 610 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 78 - protein, 524 aa, synthetic construct
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 79 - protein, 638 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 80 - protein, 496 aa, synthetic construct
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 81 - protein, 657 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 82 - protein, 477 aa, synthetic construct
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 83 - protein, 707 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 84 - protein, 427 aa, synthetic construct
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 85 - DNA, 22 bp, synthetic construct
  source 1..22: mol_type = other DNA
cagtgagcgagcgagcgcgcag

SEQ ID NO: 86 - skipped

SEQ ID NO: 87 - DNA, 21 bp, synthetic construct
  source 1..21: mol_type = other DNA
gatggagttggccactccctc

SEQ ID NO: 88 - DNA, 161 bp, synthetic construct
  source 1..161: mol_type = other DNA
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 89 - DNA, 151 bp, synthetic construct
  source 1..151: mol_type = other DNA
gggcactcttccgtggtctggtggataaattcgcaagggtatcatggcggacgaccggccggatccggccgtccgccgtgatccatgcggttaccgcccgcgtgtcgaacccaggtgtgcgacgtcagacaacgggggagcgctccttttt

SEQ ID NO: 90 - DNA, 161 bp, synthetic construct
  source 1..161: mol_type = other DNA
gggcactcttccgtgatctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 91 - DNA, 161 bp, synthetic construct
  source 1..161: mol_type = other DNA
gggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctccttttt

SEQ ID NO: 92 - DNA, 393 bp, synthetic construct
  source 1..393: mol_type = other DNA
cgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtggtctggtggataaattcgcaagggtatcatggcggacgaccggccggatccggccgtccgccgtgatccatgcggttaccgcccgcgtgtcgaacccaggtgtgcgacgtcagacaacgggg

SEQ ID NO: 93 - DNA, 417 bp, synthetic construct
  source 1..417: mol_type = other DNA
cgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 94 - DNA, 428 bp, synthetic construct
  source 1..428: mol_type = other DNA
ttcactagaatcgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctccttttt

SEQ ID NO: 95 - DNA, 11051 bp, synthetic construct
  source 1..11051: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgggcactcttccgtggtctggtggataaattcgcaagggtatcatggcggacgaaccggtaccgaaccccggatccggccgtccgccgtgatccatgcggttaccgcccgcgtgtcgaacccaggtgtgcgacgtcagacaacgggggagcgctcctttttcatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 96 - DNA, 11051 bp, synthetic construct
  source 1..11051: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgggcactcttccgtggtctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttcatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 97 - DNA, 6770 bp, synthetic construct
  source 1..6770: mol_type = other DNA
ccgcggccgccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagcaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgcctcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgggcgcg

SEQ ID NO: 98 - DNA, 6770 bp, synthetic construct
  source 1..6770: mol_type = other DNA
ccgcggccgccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaacaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgcctcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgggcgcg

SEQ ID NO: 99 - DNA, 11646 bp, synthetic construct; identical to SEQ ID NO: 3
  source 1..11646: mol_type = other DNA

SEQ ID NO: 100 - DNA, 12651 bp, synthetic construct
  source 1..12651: mol_type = other DNA
ggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatatcaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 101 - DNA, 7050 bp, synthetic construct
  source 1..7050: mol_type = other DNA
ttaaaggggtcgggtaaggtatcgggttccgataggtctggtggttctgtattccccggtgctgtccggggcaaagtccacaaactgggggtcgttgtagttgtttgtgtactggatctctgggttccacctcttggagttttccttcttgagctcccactccatctccacggtgacctgcccggtgctgtactgggtgatgaagctgctgacgggcacgtccgagaagctggtgatatttccgggcacaggcgtgttcttgatgagcatcatgggcggtgggtgtttgagtccgaatccgcccatggccggagaggggtgaaagtgcgcccccgtctctgggatcttggcccagatgggtccttggaggtacacgtccctctccatccacacgctgccgggcacgatttcctggaggttgtacgtgccggtcgcgggggcagtggtggagctctggttgttggtggccatctgcccgccgacgttgtacgccacgcggttcaccggctgcgtctcgctctcgctggtgatgagcatgttgccctcgaggtacgtggcggtggtgcccgggttcgccggctggctgttgaagatcatagtgttctccagggcataggtgttgctgccctggaggttgttggtcatgccgttcggctgcgggggcacctggtaactcgcgccctcgagctccatcctattggtcgtggcgaaggcgctgacactggcgcggttgaccccggagcccaggttccagccctgggttcggcccatgggccccgggaaccagtttttgtaggtgttggcgtatctcccggccaggttcttgttgaactggactccgccagtgttatttgtgctcacgaagcggtacaagtactggtccaccagcgggttggccagcttgaacaggttctgactgggagcgaagctggagtggaagggcacctcctcaaagttgtaggtaaactcaaagttgttgcccgttctcagcatcttgctgggaaagtactctaggcagaagaagctgctcctctcggtgggattttctgtgttgtcgcggttcagcgtcgcgtaaccgtactgcggcagcgtaaagacctgcggagggaaggccggcaggcatccctcggtcccgttgccgacgacgtagggcagctggtagtcgtcgtccgtaaacacttggacggtggaggtgaggttgttggcgatggtggtggtggagtcctgcaccgtgacctctttgacttgaatgttgaagattttgactctgagggaccggggtctgaagccccagtagttgttgatgagtctttgccagtctcgggggctccagtggctgtggaagcggttaaagtcaaagtacccccagggggtgctgtatccaaagtaggcgttggcgttgcttccgtcgacggagccgcttttgatctctcggtactggtggttgttgtagctgggcagcacccaggttcgggtggacttggtgacgactctgtcccccatccacgtggaatcgcaatgccaatctcccgaggcattgcccactccatcggcaccttggttattgtcgcccaatgggccgccacctcccgcagacattgtatcagctcccaaacttgaggctggttgggctgggatttgcagctgctgggatccgctgggtccagcttcggcgtctgacgaggtggaaggcttggagtcctcttcggtccgagccttctttctttttggaaagtggtcgtctatccgctttccggtaggggccgtcttagcaccctcttcaaccaggccaaaaggttcgagaacccttttcttggcctgaaagactgcctttccgaggtttcccccgaaggatgtgtcgtcggcgagcttctcctgaaactcggcgtccgcgtggttgtacttgaggtaggggttgtctcccgcctcaagctgctcgttgtacgagatgtcgtgctctcgcgcgacctcgtctgccctgttgacaggctctcctcgatcgagaccgtttccgggtccgagatagttataaccaggcagcacaagaccacgggcttgatcttgatgctgctgattgggttttggtttcggtgggcccgcttcaaggcccaaaaactcgcgaagaccttcaccaacttcttccaaccaatctggagggtgatcaacaaaagacatacctgatttaaatcatttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaacttttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcgagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttatttagcggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaataactagcta

SEQ ID NO: 102 - DNA, 5746 bp, synthetic construct
  source 1..5746: mol_type = other DNA
ttaaaggggtcgggtaaggtatcgggttccgataggtctggtggttctgtattccccggtgctgtccggggcaaagtccacaaactgggggtcgttgtagttgtttgtgtactggatctctgggttccacctcttggagttttccttcttgagctcccactccatctccacggtgacctgcccggtgctgtactgggtgatgaagctgctgacgggcacgtccgagaagctggtgatatttccgggcacaggcgtgttcttgatgagcatcatgggcggtgggtgtttgagtccgaatccgcccatggccggagaggggtgaaagtgcgcccccgtctctgggatcttggcccagatgggtccttggaggtacacgtccctctccatccacacgctgccgggcacgatttcctggaggttgtacgtgccggtcgcgggggcagtggtggagctctggttgttggtggccatctgcccgccgacgttgtacgccacgcggttcaccggctgcgtctcgctctcgctggtgatgagcatgttgccctcgaggtacgtggcggtggtgcccgggttcgccggctggctgttgaagatcatagtgttctccagggcataggtgttgctgccctggaggttgttggtcatgccgttcggctgcgggggcacctggtaactcgcgccctcgagctccatcctattggtcgtggcgaaggcgctgacactggcgcggttgaccccggagcccaggttccagccctgggttcggcccatgggccccgggaaccagtttttgtaggtgttggcgtatctcccggccaggttcttgttgaactggactccgccagtgttatttgtgctcacgaagcggtacaagtactggtccaccagcgggttggccagcttgaacaggttctgactgggagcgaagctggagtggaagggcacctcctcaaagttgtaggtaaactcaaagttgttgcccgttctcagcatcttgctgggaaagtactctaggcagaagaagctgctcctctcggtgggattttctgtgttgtcgcggttcagcgtcgcgtaaccgtactgcggcagcgtaaagacctgcggagggaaggccggcaggcatccctcggtcccgttgccgacgacgtagggcagctggtagtcgtcgtccgtaaacacttggacggtggaggtgaggttgttggcgatggtggtggtggagtcctgcaccgtgacctctttgacttgaatgttgaagattttgactctgagggaccggggtctgaagccccagtagttgttgatgagtctttgccagtctcgggggctccagtggctgtggaagcggttaaagtcaaagtacccccagggggtgctgtatccaaagtaggcgttggcgttgcttccgtcgacggagccgcttttgatctctcggtactggtggttgttgtagctgggcagcacccaggttcgggtggacttggtgacgactctgtcccccatccacgtggaatcgcaatgccaatctcccgaggcattgcccactccatcggcaccttggttattgtcgcccaatgggccgccacctcccgcagacattgtatcagctcccaaacttgaggctggttgggctgggatttgcagctgctgggatccgctgggtccagcttcggcgtctgacgaggtggaaggcttggagtcctcttcggtccgagccttctttctttttggaaagtggtcgtctatccgctttccggtaggggccgtcttagcaccctcttcaaccaggccaaaaggttcgagaacccttttcttggcctgaaagactgcctttccgaggtttcccccgaaggatgtgtcgtcggcgagcttctcctgaaactcggcgtccgcgtggttgtacttgaggtaggggttgtctcccgcctcaagctgctcgttgtacgagatgtcgtgctctcgcgcgacctcgtctgccctgttgacaggctctcctcgatcgagaccgtttccgggtccgagatagttataaccaggcagcacaagaccacgggcttgatcttgatgctgctgattgggttttggtttcggtgggcccgcttcaaggcccaaaaactcgcgaagaccttcaccaacttcttccaaccaatctggagggtgatcaacaaaagacatacctgatttaaatcatttattgttcaaagatgcagtcatccaaatccacattgaccagatcgcaggcagtgcaagcgtctggcacctttcccatgatatgatgaatgtagcacagtttctgatacgcctttttgacgacagaaacgggttgagattctgacacgggaaagcactctaaacagtctttctgtccgtgagtgaagcagatatttgaattctgattcattctctcgcattgtctgcagggaaacagcatcagattcatgcccacgtgacgagaacatttgttttggtacctgtctgcgtagttgatcgaagcttccgcgtctgacgtcgatggctgcgcaacttttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcgagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttatttagcggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaataactagcta

SEQ ID NO: 103 - DNA, 154 bp, synthetic construct
  source 1..154: mol_type = other DNA
aataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 104 - DNA, 8189 bp, synthetic construct
  source 1..8189: mol_type = other DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctactaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggatcctgccattgcatccttctctgcagactaagatggagttcctgaaccaagaccgcttgctggccaacctgtgaaactgggctcaaggtgaggggtgctatctgtgattgagggacatggttaatggaattgtctcacacagaaatcgcacccgtcaccttggcctacttatcaccaccccaaacagaggaacacgccttctccagccacagcctatggaagggccttcagctgctgtggccccgaggtaagcttgggggaggctaactgaaacacggaaggagacaataccggaaggaacccgcgctatgacggcaataaaaagacagaataaaacgcacggtgttgggtcgttgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 105 - DNA, 11635 bp, synthetic construct
  source 1..11635: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 106 - DNA, 10644 bp, synthetic construct
  source 1..10644: mol_type = other DNA
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatatcaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 107 - DNA, 7124 bp, synthetic construct
  source 1..7124: mol_type = other DNA
cagaggtgtaaagtacttgagtaattttacttgattactgtacttaagtattatttttggggatttttactttacttgagtacaattaaaaatcaatacttttacttttacttaattacatttttttagaaaaaaaagtactttttactccttacaattttatttacagtcaaaaagtacttattttttggagatcacttcattctattttcccttgctattaccaaaccaattgaattgcgctgatgcccagtttaatttatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatatcaagcttaaacaagaatctctagttttctttcttgcttttacttttacttccttaatactcaagtacaattttaatggagtacttttttacttttactcaagtaagattctagccagatacttttacttttaattgagtaaaattttccctaagtacttgtactttcacttgagtaaaatttttgagtactttttacacctctg

SEQ ID NO: 108 - DNA, 8271 bp, synthetic construct; identical to SEQ ID NO: 15
  source 1..8271: mol_type = other DNA

SEQ ID NO: 109 - DNA, 8567 bp, synthetic construct; identical to SEQ ID NO: 39
  source 1..8567: mol_type = other DNA

SEQ ID NO: 110 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 111 - DNA, 222 bp, synthetic construct
  source 1..222: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaacacgtgcggaccgagtggcgtaatcat

SEQ ID NO: 112 - DNA, 245 bp, synthetic construct
  source 1..245: mol_type = other DNA
ttgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtg

SEQ ID NO: 113 - DNA, 56 bp, synthetic construct
  source 1..56: mol_type = other DNA
aataaaggaaatttattttcattgcaatagtgtgttggaattttttgtgtctctca

SEQ ID NO: 114 - DNA, 3952 bp, synthetic construct; identical to SEQ ID NO: 22
  source 1..3952: mol_type = other DNA

SEQ ID NO: 115 - DNA, 222 bp, synthetic construct; identical to SEQ ID NO: 111
  source 1..222: mol_type = other DNA

SEQ ID NO: 116 - DNA, 10 bp, synthetic construct
  source 1..10: mol_type = other DNA
aaaaaggggg

SEQ ID NO: 117 - DNA, 10 bp, synthetic construct
  source 1..10: mol_type = other DNA
aaaaaaaaaa

SEQ ID NO: 118 - DNA, 20 bp, synthetic construct
  source 1..20: mol_type = other DNA
cccccggggggggggccccc

SEQ ID NO: 119 - DNA, 10 bp, synthetic construct
  source 1..10: mol_type = other DNA
gggggggggg

SEQ ID NO: 120 - protein, 10 aa, synthetic construct
FFFFFYYYYY

SEQ ID NO: 121 - protein, 10 aa, synthetic construct
YYYYYYYYYY

SEQ ID NO: 122 - protein, 20 aa, synthetic construct
LLLLLFFFFFYYYYYLLLLL

SEQ ID NO: 123 - DNA, 10 bp, synthetic construct
  source 1..10: mol_type = other DNA
cccccttttt